(19) United States
(12) Patent Application Publication
(10) Pub. No.: US 2009/0270417 A1
(43) Pub. Date: Oct. 29, 2009

(54) Organic Compounds

(75) Inventors: Juergen Klaus Maibaum, Weil-Haltingen (DE); Edwige Liliane Jeanne Lorthiois, Niffer (FR); Peter Buehimayer, Hangstrasse (CH)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG, Basel (CH)

(21) Appl. No.: 12/304,389

(22) PCT Filed: Jun. 11, 2007
(86) PCT No.: PCT/EP2007/005130

§ 371 Date: Dec. 11, 2008

(30) Foreign Application Priority Data
Jun. 13, 2006 (GB) 0611697.4

(51) Int. Cl. A61K 31/40; C07D 207/09; C07D 405/12; C07D 403/12; A61K 31/4025; A61K 31/497; A61P 9/12

(52) U.S. Cl. 514/255.05; 548/568; 548/517; 544/406; 514/428; 514/422

(57) ABSTRACT

Novel 3,4-di-, 3,3,4-di-, 3,4,4,-tri- and 3,3,4,4-tetra-substituted pyrrolidine compounds, these compounds for use in the diagnostic and therapeutic treatment of a warm-blooded animal, especially for the treatment of a disease (=disorder) that depends on inappropriate activity of renin; the use of a compound of that class for the preparation of a pharmaceutical formulation for the treatment of a disease that depends on inappropriate activity of renin; the use of a compound of that class in the treatment of a disease that depends on inappropriate activity of renin; pharmaceutical formulations comprising a said substituted pyrrolidine compound, and/or a method of treatment comprising administering a said substituted pyrrolidine compound, a method for the manufacture of said substituted pyrrolidine compounds, and novel intermediates and partial steps for their synthesis are described. The substituted pyrrolidine compounds are especially of the formula (I) wherein the substituents are as described in the specification.

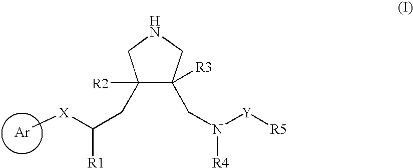

The invention relates to (3,4-di-, 3,3,4-tri, 3,4,4-tri- or 3,3,4,4-tetra-)substituted pyrrolidine compounds, these compounds for use in the diagnostic and therapeutic treatment of a warm-blooded animal, especially for the treatment of a disease (=disorder) that depends on activity of renin; the use of a compound of that class for the preparation of a pharmaceutical formulation for the treatment of a disease that depends on activity of renin; the use of a compound of that class in the treatment of a disease that depends on activity of renin; pharmaceutical formulations comprising said substituted pyrrolidine compound, and/or a method of treatment comprising administering said substituted pyrrolidine compound, a method for the manufacture of said substituted pyrrolidine compound, and novel intermediates and partial steps for its synthesis.

The present invention provides especially compounds of the formula I

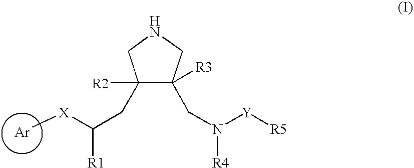

wherein
R¹ is unsubstituted or substituted alkyl or substituted or unsubstituted cycloalkyl;
R² and R³ are independently of each other hydrogen, alkoxy, alkyl, hydroxy or halogen;
R⁴ is unsubstituted or substituted alkyl or substituted or unsubstituted cycloalkyl;
R⁵ is unsubstituted or substituted alkyl, substituted or unsubstituted heterocyclyl, unsubstituted or substituted or unsubstituted aryl, or substituted or unsubstituted cycloalkyl;

### X is CH₂ or O;

### Y is —(CO)—, —S(O)₂— or —C(O)O—; and

Ar is unsubstituted or substituted aryl or unsubstituted or substituted mono- or bicyclic aromatic heterocyclyl;
or a salt thereof.

The compounds of the present invention exhibit inhibitory activity on the natural enzyme renin. Thus, compounds of formula I may be employed for the treatment (this term also including prophylaxis) of one or more disorders or diseases selected from, inter alia, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

Listed below are definitions of various terms used to describe the compounds of the present invention as well as their use and synthesis, starting materials and intermediates and the like. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group.

The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo. If not explicitly or implicitly stated otherwise, halo can also stand for more than one halogen substitutent in moieties such as alkyl, alkanoyl and the like (e.g. in trifluoromethyl, trifluoroacetyl).

Unsubstituted or substituted aryl preferably is a is mono- or polycyclic, especially monocyclic, bicyclic, tricyclic aryl with 6 to 22 carbon atoms, especially phenyl, naphthyl, indenyl or fluorenyl, and is unsubstituted or substituted by one or more, especially one to three, moieties, preferably independently selected from the group consisting of:
a substitutent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, or is phenyl, naphthyl, phenyl- or naphthyl-C₁-C₇-alkyl and halo-C₁-C₇-alkyl; where said substituent —(C₀-C₇-alkylene)-(X)ᵣ—(C₁-C₇-alkylene)-(Y)ₛ—(C₀-C₇-alkylene)-His preferably C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, such as aminomethyl, (N-) mono- or (N,N-) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇alkoxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl and N-mono- or N,N-di-(C₁-C₇-alkyl)-aminosulfonyl;
C₂-C₇-alkenyl, C₂-C₇-alkinyl, phenyl, naphtyl, heterocyclyl, especially as defined below for heterocyclyl, preferably selected from pyrrolyl, furanyl, thienyl, pyrimidine-2,4-dione-1-, -3- or -5-yl and benzo[1,3]-dioxolyl, phenyl- or naphthyl- or heterocyclyl-C₁-C₇-alkyl wherein heterocyclyl is as defined below, preferably selected from pyrrolyl, furanyl, thienyl and benzo[1,3]-dioxolyl; such as benzyl or naphthylmethyl, halo-C₁-C₇-alkyl, such as trifluoromethyl, phenyloxy- or naphthyloxy-C₁-C₇-alkyl, phenyl-C₁-C₇-alkoxy- or naphthyl-C₁-C₇-alkoxy-C₁-C₇-alkyl, di-(naphthyl- or phenyl)-amino-C₁-C₇-alkyl, di-(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, carboxy-C₁-C₇-alkyl, halo, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, phenyl- or naphthyloxy, phenyl- or naphthyl-C₁-C₇-alkyloxy, benzoyl- or naphthoyloxy, halo-C₁-C₇-alkylthio, such as trifluoromethylthio, phenyl- or naphthylthio, phenyl- or naphthyl-C₁-C₇-alkylthio, benzoyl- or naphthoylthio, nitro, amino, di-(naphthyl- or phenyl-C₁-C₇-alkyl)-amino, benzoyl- or naphthoylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N-) mono- or (N,N-) di-(C₁-C₇-alkyl)-amino-C₁-C₇-alkylcarbonyl, C₁-C₇-alkanoylamino-C₁-C₇-alkylcarbonyl, halo-C₁-C₇-alkoxycarbonyl, phenyl- or naphthyloxycarbonyl, phenyl- or naphthyl-C₁-C₇-alkoxycarbonyl, (N,N-) di-(C₁-C₇-alkyl)-amino-C₁-C₇-alkoxycarbonyl, carbamoyl, N-mono or N,N-di-(naphthyl- or phenyl-)-aminocarbonyl, N-mono- or N,N-di-(naphthyl- or phenyl-C₁-C₇-alkyl)-aminocarbonyl, cyano, C₁-C₇-alkylene which is unsubstituted or substituted by up to four C₁-C₇-alkyl substituents and bound to two adjacent ring atoms of the aryl moiety, C₂-C₇-alkenylene or -alkinylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N-) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)-aminosulfonyl.

Unsubstituted or substituted heterocyclyl is a mono- or bicyclic, unsaturated, partially saturated or saturated ring system with preferably 3 to 22 (more preferably 3 to 14) ring atoms and with one or more, preferably one to four, heteroatoms independently selected from nitrogen (═N—, —NH— or substituted —NH—), oxygen, sulfur (—S—, S(═O)— or S—(═O)₂—) which is unsubstituted or substituted by one or more, e.g. up to three, substitutents preferably independently selected from the substitutents mentioned above for aryl and from oxo. Preferably, unsubstituted or substituted heterocyclyl is selected from the following moieties:

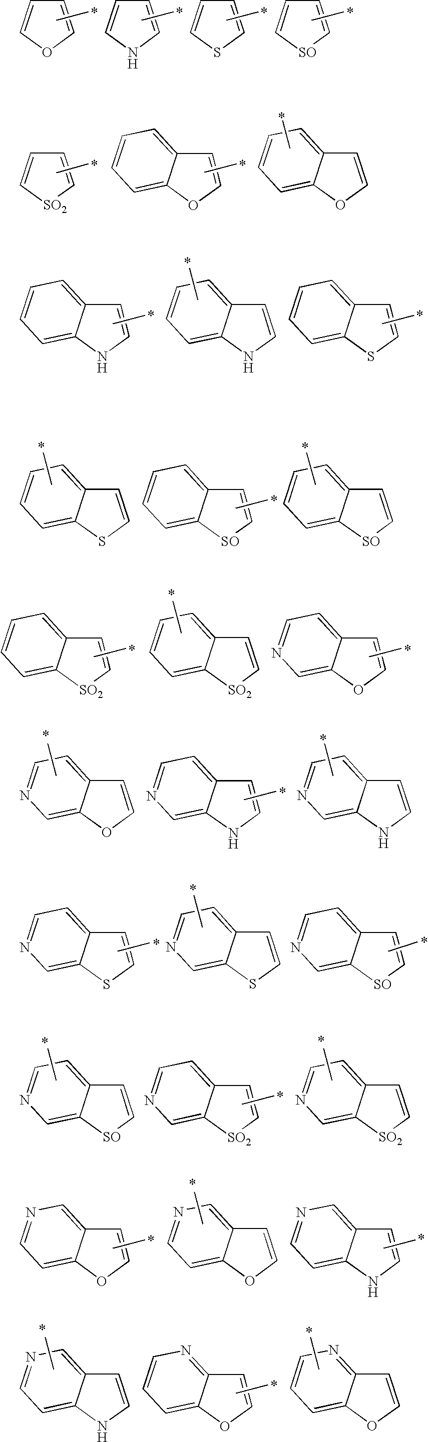

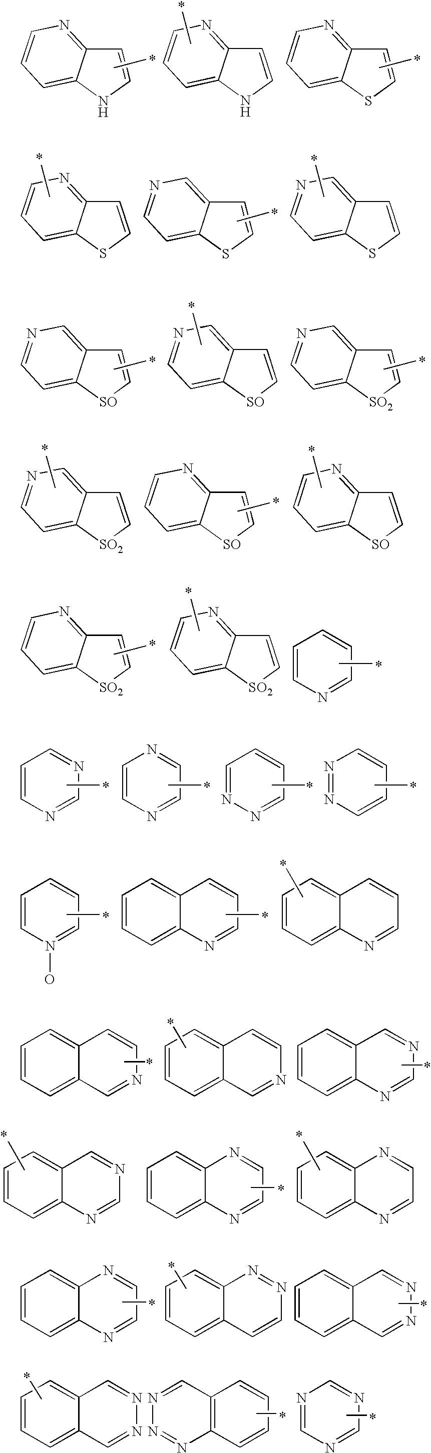

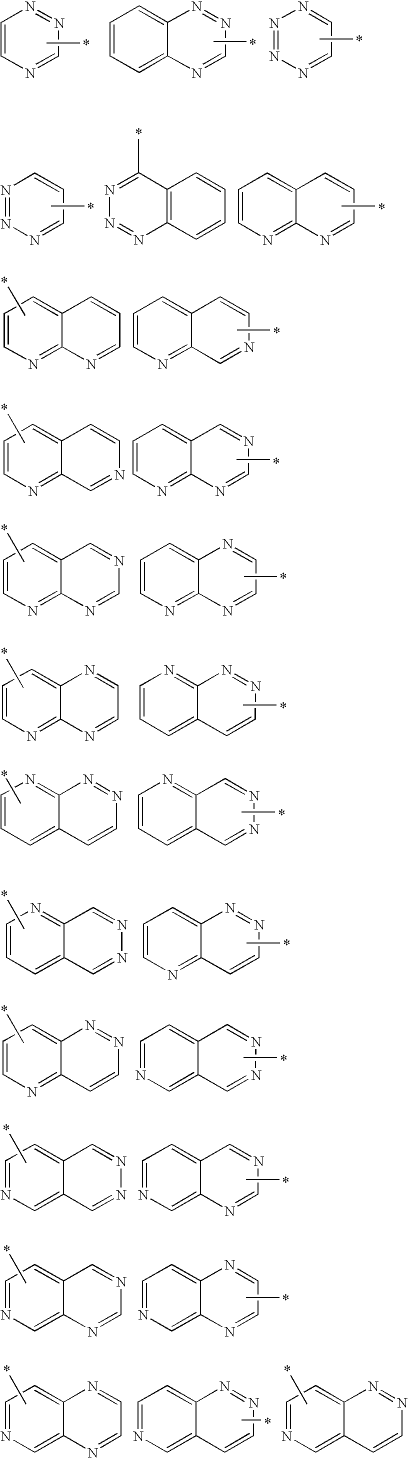

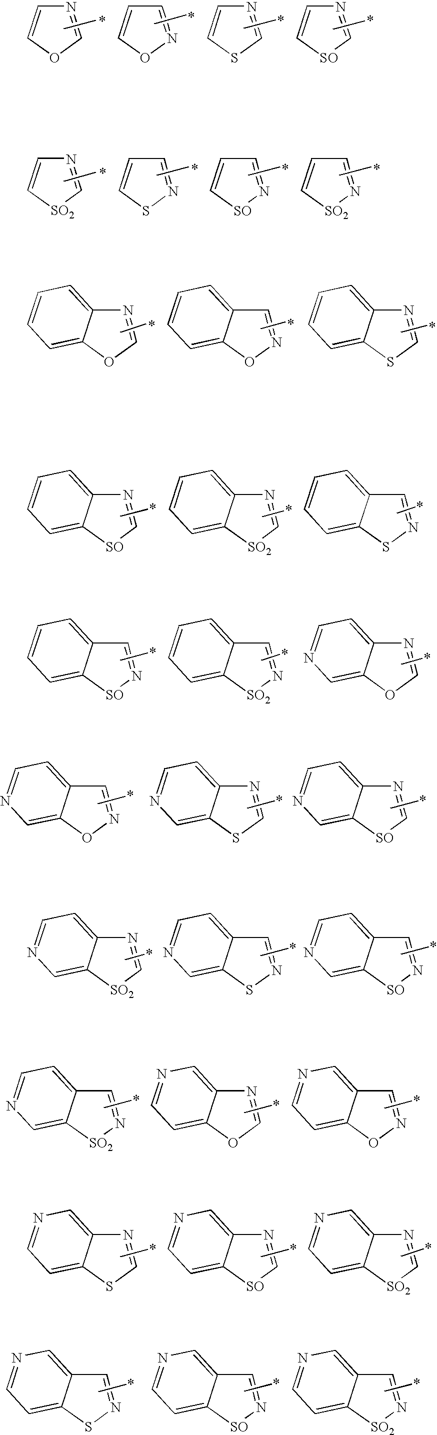

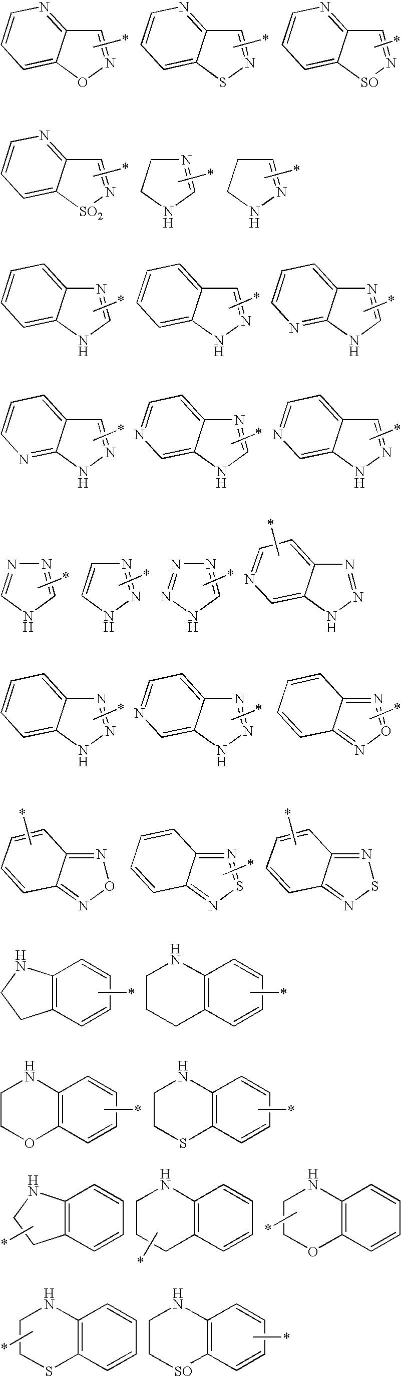

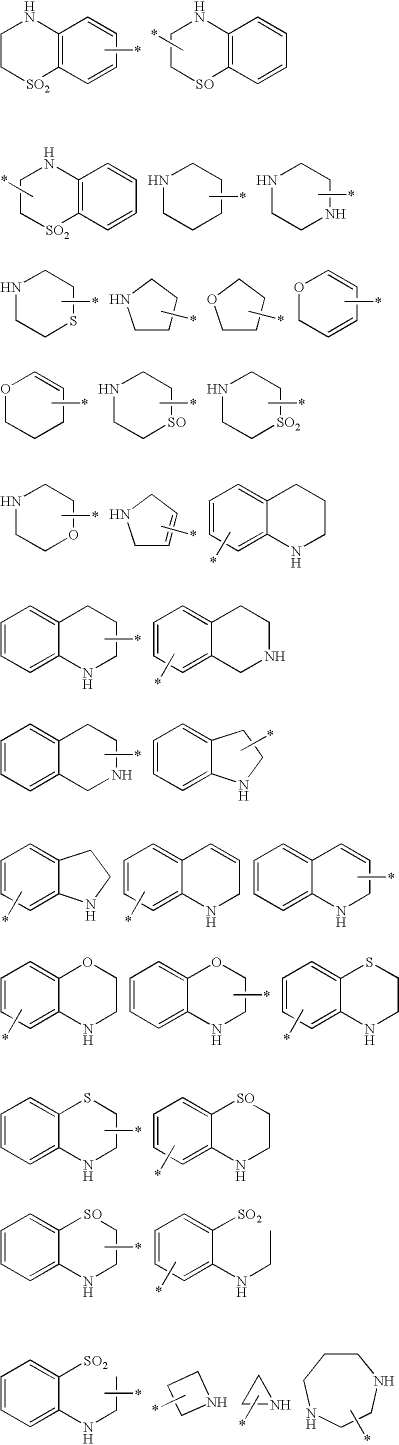

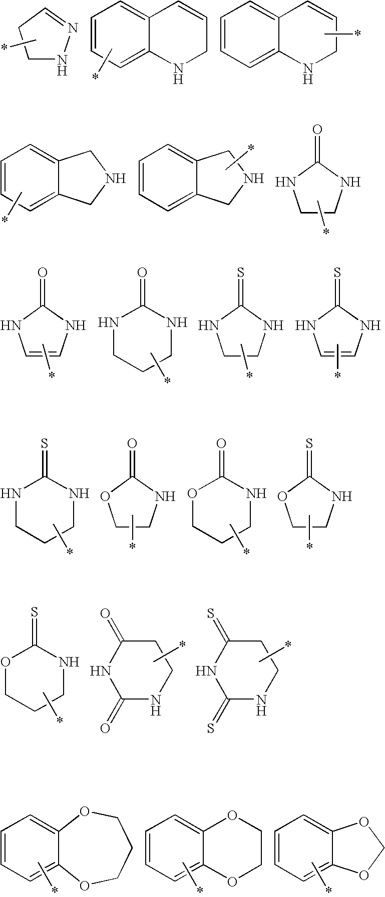

where in each case where an NH is present the bond with the asterisk connecting the respective heterocyclyl moiety to the rest of the molecule the H may be replaced with said bond and/or the H may be replaced by a substituent, and one or more substituents may be present as just described.

Unsubstituted or substituted cycloalkyl is preferably mono- or polycyclic, more preferably monocyclic, C₃-C₁₀-cycloalkyl which may include one or more double (e.g. in cycloalkenyl) and/or triple bonds (e.g. in cycloalkinyl), and is unsubstituted or substituted by one or more, e.g. one to three substitutents preferably independently selected from those mentioned above as substituents for aryl.

Unsubstituted or substituted alkyl is preferably C₁-C₂₀-alkyl, more preferably C₁-C₇-alkyl, that is straight-chained or branched (one or, where appropriate, more times), which is unsubstituted or substituted by one or more, e.g. up to three moieties selected from unsubstituted or substituted aryl as described above, especially phenyl or naphthyl each of which is unsubstituted or substituted as described above for unsubstituted or substituted aryl, unsubstituted or substituted heterocyclyl as described above, especially pyrrolyl, furanyl, thienyl, pyrimidine-2,4-dione-1-, -2-, -3- or -5-yl or benzo[1,3]dioxolyl, which heterocyclyl is unsubstituted or substituted as described above for unsubstituted or substituted heterocyclyl; unsubstituted or substituted cycloalkyl as described above, especially cyclopropyl, cyclobutyl, cyclopentyl or cyclohexyl each of which is unsubstituted or substituted as described above for unsubstituted or substituted cycloalkyl; C₂-C₇-alkenyl, C₂-C₇-alkinyl, halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkylthio, such as trifluoromethylthio, hydroxy-C₁-C₇-alkylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl, hydroxy-C₁-C₇-alkyl and/or C₁-C₇-alkoxy-C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl)-aminocarbonyl, N-mono- or N,N-di-(naphthyl- or phenyl-C₁-C₇-alkyl)-aminocarbonyl, cyano, C₁-C₇-alkenylene or -alkinylene, C₁-C₇-alkylenedioxy, sulfenyl, (—S—OH) sulfonyl (—S(═O)—OH), C₁-C₇-alkylsulfinyl (C₁-C₇-alkyl-S(═O)—), phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl, C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl, N-mono or N,N-di-(C₁-C₇-alkyl, phenyl-, naphthyl, phenyl-C₁-C₇-alkyl or naphthyl-C₁-C₇-alkyl)-aminosulfonyl, N-mono-, N′-mono-, N,N-di- or N,N,N′-tri-(C₁-C₇-alkyl, hydroxy-C₁-C₇-alkyl and/or C₁-C₇-alkoxy-C₁-C₇-alkyl)-aminocarbonylamino and N-mono-, N′-mono-, N,N-di- or N,N,N′-tri-(C₁-C₇-alkyl, hydroxy-C₁-C₇-alkyl and/or C₁-C₇-alkoxy-C₁-C₇-alkyl)aminosulfonylamino. In cases where unsubstituted or substituted heterocyclyl-alkyl, unsubstituted or substituted aryl-alkyl or unsubstituted or substituted cycloalkyl-alkyl-moieties are mentioned as substituents, the definition of unsubstituted or substituted alkyl relates to such moieties which, in addition to unsubstituted or substituted heterocyclyl, aryl or cycloalkyl comprise at least one further and different moiety (especially from those mentioned in this paragraph) as alkyl substitutent.

In substituted or unsubstituted alkylsulfonyl, substituted or unsubstituted alkyl is preferably as defined above for unsubstituted or substituted alkyl.

In substituted or unsubstituted arylsulfonyl, substituted or unsubstituted aryl is preferably as defined above for unsubstituted or substituted aryl.

In substituted or unsubstituted heterocyclylsulfonyl, substituted or unsubstituted heterocyclyl is preferably as defined above for unsubstituted or substituted heterocyclyl.

In substituted or unsubstituted cycloalkylsulfonyl, unsubstituted or substituted cycloalkyl is preferably as defined above for unsubstituted or substituted cycloalkyl.

In all definitions above it goes without saying that only stable compounds the person having skill in the art will, without undue experimentation or considerations, be able to recognize are important (e.g. those that are sufficiently stable for the manufacture of pharmaceuticals, e.g. having a half-life of more than 30 seconds) and thus are preferably encompassed by the present claims and that only chemically feasible bonds and substitutions are encompassed, as well as tautomeric forms where present.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous solutions, or can be isolated especially in solid form.

Such salts are formed, for example, as acid addition salts, preferably with organic or inorganic acids, from compounds of formula I with a basic nitrogen atom (e.g. imino or amino), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-di-methylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds” and “intermediates” hereinbefore and hereinafter, especially to the compound(s) of the formula I, is to be understood as referring also to one or more salts thereof or a mixture of a free compound and one or more salts thereof, each of which is intended to include also any solvate, metabolic precursor such as ester or amide of the compound of formula I, or salt of any one or more of these, as appropriate and expedient and if not explicitly mentioned otherwise. Different crystal forms may be obtainable and then are also included.

Where the plural form is used for compounds, salts, pharmaceutical preparations, diseases, disorders and the like, this is intended to mean one (preferred) or more single compound(s), salt(s), pharmaceutical preparation(s), disease(s), disorder(s) or the like, where the singular or the indefinite article (“a”, “an”) is used, this is intended to include the plural or preferably the singular.

The compounds of the present invention possess two or more asymmetric centers depending on the choice of the substituents. The preferred absolute configuration at the C-3 and C-4 asymmetric centers is maintained throughout the specification and the appended claims as indicated herein-above. However, any possible diastereoisomers, enantiomers and geometric isomers, and mixtures thereof, e.g., racemates, are encompassed by the present invention.

As described herein above, the present invention provides 3,4-disubstituted pyrrolidine derivatives of formula I, these compounds for use in the (prophylactic and/or therapeutic) treatment of a disease (=condition, disorder) in a warm-blooded animal, especially a human, preferably of a disease dependent on (especially inappropriate) renin activity, a pharmaceutical composition comprising a compound of the formula I, methods for preparing said compound or pharmaceutical preparation, and methods of treating conditions dependent on (especially inappropriate) renin activity by administration of a therapeutically effective amount of a compound of the formula I, or a pharmaceutical composition thereof.

“Inappropriate” renin activity preferably relates to a state of a warm-blooded animal, especially a human, where renin shows a renin activity that is too high in the given situation (e.g. due to one or more of misregulation, overexpression e.g. due to gene amplification or chromosome rearrangement or infection by microorganisms such as virus that express an aberrant gene, abnormal activity e.g. leading to an erroneous substrate specificity or a hyperactive renin e.g. produced in normal amounts, too low activity of renin activity product removing pathways, high substrate concentration, other circumstances that make the activity of renin relatively too high, such as other mechanisms leading to blood pressure increase, and/or the like) and/or leads to or supports a renin dependent disease or disorder as mentioned above and below, e.g. by renin activity the reduction of which has beneficial effects in the given disease. Such inappropriate renin activity may, for example, comprise a higher than normal activity, or further an activity in the normal or even below the normal range which, however, due to preceding, parallel and or subsequent processes, e.g. signaling, regulatory effect on other processes, higher substrate or product concentration and the like, leads to direct or indirect support or maintenance of a disease or disorder, and/or an activity that supports the outbreak and/or presence of a disease or disorder in any other way. The inappropriate activity of renin may or may not be dependent on parallel other mechanisms supporting the disorder or disease, and/or the prophylactic or therapeutic effect may or may include other mechanisms in addition to inhibition of renin. Therefore “dependent” has to be read as “dependent inter alia”, (especially in cases where a disease or disorder is really exclusively dependent only on renin) preferably as “dependent mainly”, more preferably as “dependent essentially only”.

Where a disease or disorder dependent on inappropriate activity of a renin is mentioned (such in the definition of “use” in the following paragraph and also especially where a compound of the formula I is mentioned for use in the diagnostic or therapeutic treatment which is preferably the treatment of a disease or disorder dependent on inappropriate renin activity, this refers preferably to any one or more diseases or disorders that depend on inappropriate activity of natural renin and/or one or more altered or mutated forms (including alleles or single nuclear polymorphism forms thereof).

Where subsequently or above the term “use” is mentioned (as verb or noun) (relating to the use of a compound of the formula I or of a pharmaceutically acceptable salt thereof, or a method of use thereof), this (if not indicated differently or to be read differently in the context) includes any one or more of the following embodiments of the invention, respectively (if not stated otherwise): the use in the treatment of a disease or disorder that depends on (especially inappropriate) activity of renin, the use for the manufacture of pharmaceutical compositions for use in the treatment of a disease or disorder that depends on (especially inappropriate) activity of renin; a method of use of one or more compounds of the formula I in the treatment of a disease or disorder that depends on (especially inappropriate) activity of renin; a pharmaceutical preparation comprising one or more compounds of the formula I for the treatment of a disease or disorder that depends on (especially inappropriate) activity of renin; and one or more compounds of the formula I for use in the treatment of a disease or disorder in a warm-blooded animal, especially a human, preferably a disease that depends on (especially inappropriate) activity of renin; as appropriate and expedient, if not stated otherwise.

The terms “treat”, “treatment” or “therapy” refer to the prophylactic (e.g. delaying or preventing the onset of a disease or disorder) or preferably therapeutic (including but not limited to preventive, delay of onset and/or progression, palliative, curing, symptom-alleviating, symptom-reducing, patient condition ameliorating, renin-modulating and/or renin-inhibiting) treatment of said disease(s) or disorder(s), especially of the one or more disease or disorder mentioned above or below.

### PREFERRED EMBODIMENTS ACCORDING TO THE INVENTION

The groups of preferred embodiments of the invention mentioned below are not to be regarded as exclusive, rather, e.g., in order to replace general expressions or symbols with more specific definitions, parts of those groups of compounds can be interchanged or exchanged using the definitions given above, or omitted, as appropriate.

Highly preferred is a compound of the formula IA with the following configuration:

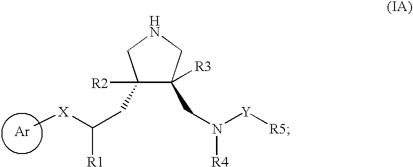

Preferred is a compound of the formula IB with the following configuration:

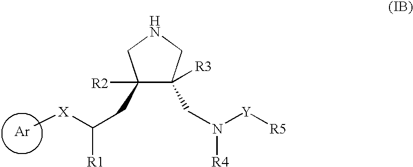

Preferred is also a compound of the formula IC with the following configuration:

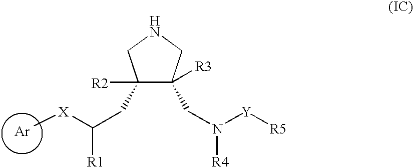

Preferred is also a compound of the formula ID with the following configuration:

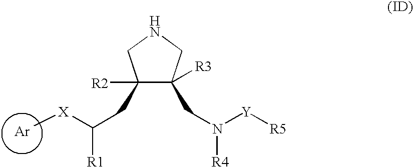

Preferred is also a compound of the formula IE with the following configuration:

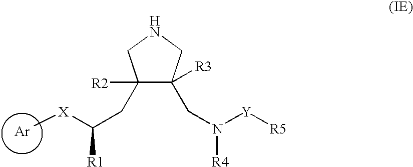

Most preferred is a compound of the formula IF with the following configuration:

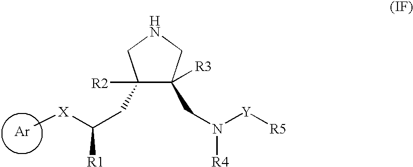

In each of the formulae IA, IB, IC, ID, IE and IF, the moieties R¹, R², R³, R⁴, R⁵, X, Y and Ar are as defined hereinbefore or preferably hereinafter.

The formula IA, IB, IC, ID, IE or IF can replace formula I wherever a compound of the formula I (including a salt thereof) is mentioned hereinbefore or hereinafter; also, the corresponding intermediates are preferred.

The following preferred embodiments of the moieties and symbols in formula I can be employed independently of each other to replace more general definitions and thus to define specially preferred embodiments of the invention, where the remaining definitions can be kept broad as defined in embodiments of the inventions defined above of below.

### Preferred Definitions for R1

R1 is preferably unsubstituted or substituted alkyl or substituted or unsubstituted cycloalkyl, whereby suitable substitutents include O—C₁-C₄-alkyl, halo, hydroxy, unsubstituted or substituted, preferably substituted, phenyl, unsubstituted or substituted, preferably substituted, naphthyl, unsubstituted or substituted, preferably substituted, phenyl- or naphthyloxy, unsubstituted or substituted, preferably substituted, phenyl- or naphthyl-C₁-C₇-alkyloxy, unsubstituted or substituted, preferably substituted, heterocyclyl, unsubstituted or substituted, preferably unsubstituted, cycloalkyl, nitro, amino, amino-C₁-C₇-alkyl, N-mono- or N,N-di-substituted aminocarbonyl, carboxyl, and cyano. More preferably R¹ is unsubstituted.

In one embodiment, R1 is preferably C₁-C₇-alkyl, more preferably C₁-C₄-alkyl, such as methyl, ethyl, n-propyl, isopropyl, n-butyl, isobutyl, sec-butyl and tert-butyl, most preferably isopropyl.

In another preferred embodiment, R1 is preferably C₃-C₁₀-cycloalkyl, more preferably C₃-C₇-cycloalkyl, still more preferably C₃-, C₄-, C₅- or C₆-Cycloalkyl, most preferably cyclopropyl.

Most preferably, R1 is isopropyl.

### Preferred Definitions for R2 and R3

R2 and R3 are independently of each other preferably hydrogen, hydroxy or halogen, more preferably hydrogen or hydroxyl, most preferably hydrogen. When one of R2 and R3 are other than hydrogen, such as hydroxy or halogen, preferably hydroxyl, then the other is preferably hydrogen.

### Preferred Definitions for R4

R4 is preferably unsubstituted or substituted alkyl or substituted or unsubstituted cycloalkyl, whereby suitable substitutents include O—C₁-C₄-alkyl, halo, hydroxy, unsubstituted or substituted, preferably substituted, phenyl, unsubstituted or substituted, preferably substituted, naphthyl, unsubstituted or substituted, preferably substituted, phenyl- or naphthyloxy, unsubstituted or substituted, preferably substituted, phenyl- or naphthyl-C₁-C₇-alkyloxy, unsubstituted or substituted, preferably substituted, heterocyclyl, unsubstituted or substituted, preferably unsubstituted, cycloalkyl, nitro, amino, amino-C₁-C₇-alkyl, N-mono- or N,N-di-substituted aminocarbonyl, carboxyl, and cyano. More preferably R4 is unsubstituted.

In one embodiment, R4 is preferably C₁-C₇-alkyl, more preferably C₁-C₄-alkyl, such as methyl, ethyl, n-propyl, isopropyl, n-butyl, isobutyl, sec-butyl and tert-butyl, most preferably methyl or isopropyl.

In another preferred embodiment, R4 is preferably C₃-C₁₀-cycloalkyl, more preferably C₃-C₇-cycloalkyl, still more preferably C₃-, C₄-, C₅- or C₆-Cycloalkyl, most preferably cyclopropyl.

Most preferably, R4 is cyclopropyl.

### Preferred Definitions for Y and R⁵

In one embodiment, Y is preferably —C(O)—.

In another embodiment, Y is preferably —C(O)O—.

R⁵ is preferably unsubstituted or substituted alkyl, substituted or unsubstituted heterocyclyl, unsubstituted or substituted or unsubstituted aryl, or substituted or unsubstituted cycloalkyl, wherein each is unsubstituted or substituted by one or more, e.g. up to three, substitutents selected from the group consisting of halo, phenyl or naphthyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl, cyano, C₁-C₇alkyl and substituted or unsubstituted heterocyclyl, such as tetrahydropyranyl; more preferably selected from the group consisting of halo, phenyl or naphthyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano.

More preferably, R⁵ is C₁-C₇-alkyl or 5- to 10-membered mono- or bicyclic heterocyclyl containing at least one heteroatom selected from O, N or S, wherein each is unsubstituted or substituted by one or more, e.g. up to three, substitutents selected from the group consisting of:

halo, phenyl or naphthyl, hydroxy, C₁-C₇-alkoxy, amino, mono- or di-(C₁-C₇alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl, cyano, C₁-C₇-alkyl and substituted or unsubstituted heterocyclyl; more preferably selected from the group consisting of halo, phenyl or naphthyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl and cyano.

Even more preferably, R⁵ is methyl, isobutyl, tetrahydropyranyl or pyrazinyl, wherein each is unsubstituted or substituted by one or more, e.g. up to three, substitutents selected from the group consisting of phenyl, hydroxyl, methyl or tetrahydropyranyl. Most preferably, R⁵ is methyl, isobutyl or tetrahydropyranyl, wherein each is unsubstituted or substituted by one or more, e.g. up to three, substitutents selected from the group consisting of phenyl or hydroxyl.

In a first embodiment R⁵ is preferably unsubstituted or substituted alkyl.

Preferred examples for alkyl are branched or straight chain C₁-C₇-alkyl which may be substituted or unsubstituted. Preferred examples include methyl, ethyl, isopropyl, n-propyl, n-butyl, sec-butyl or tert-butyl, more preferably methyl, ethyl, isopropyl, or isobutyl most preferably methyl or isobutyl. The alkyl moiety is preferably substituted. When the alkyl moiety is substituted, it is preferably mono-, di- or tri-substituted, more preferably mono-substituted. Suitable substituents for the alkyl moiety are as defined herein, preferably halo, phenyl or naphthyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl; and cyano; more preferably halo, phenyl or naphthyl, hydroxy, C₁-C₇-alkoxy, nitro, carboxyl, C₁-C₇-alkoxy-carbonyl, and cyano; most preferably phenyl or hydroxyl. Whenever phenyl and naphthyl are mentioned as substituents, these may be substituted (mono-, di- or tri-substituted, preferably mono-substituted) or unsubstituted, preferably unsubstituted. Suitable substituents for phenyl or naphthyl include C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkylsulfonylamino-C₁-C₇-alkyl, carboxy-C₁-C₇-alkyl, C₁-C₇-alkoxycarbonyl-C₁-C₇-alkyl, halo, hydroxy, C₁-C₇-alkoxy, C₁-C₇-alkoxy-C₁-C₇-alkoxy, carboxy-C₁-C₇-alkoxy, amino-C₁-C₇-alkoxy, N—C₁-C₇-alkanoylamino-C₁-C₇-alkoxy, carbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carboxyl, carbamoyl and N—C₁-C₇-alkoxy-C₁-C₇-alkylcarbamoyl.

In another embodiment, when the alkyl moiety is substituted, it is preferably mono-, di- or tri-substituted, more preferably mono-substituted by unsubstituted or substituted heterocyclyl. The heterocyclyl substituent is preferably mono- or bicyclic, more preferably bicyclic. Preferred are saturated ring systems. The heterocyclyl moiety has preferably 1, 2 or 3, more preferably 1 or 2, most preferably 2, heteroatoms selected from O, N or S, more preferably O or N. Particularly preferred examples include 5- to 10-membered mono- or bicyclic heterocyclyl, such as bicyclic 9 or 10-membered rings preferably containing a nitrogen atom, in particular, quinolyl, isoquinolyl, 1,2,3,4-tetrahydro-1,4-benzoxazinyl, 2H-1,4-benzoxazin-3(4H)-only, 3,4-dihydro-1H-quinolin-2-onyl, or 4H-benzo[1,4]thiazin-3-onyl; indolyl, 1H-indazolyl, benzothiophenyl, imidazo[1,2-a]pyridyl or 3H-benzooxazol-2-only; or more preferably 5- or 6-membered monocyclic rings containing an O or N atom such as tetrahydrofuranyl, tetrahydropyranyl, furanyl, pyranyl, piperidinyl, pyrrolidinyl, imidazolyl, triazolyl, piperazinyl, morpholinyl, pyrimidinyl or pyridinyl, where each heterocyclyl is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkylsulfonylamino-C₁-C₇-alkyl, carboxy-C₁-C₇-alkyl, C₁-C₇-alkoxycarbonyl-C₁-C₇-alkyl, halo, hydroxy, C₁-C₇-alkoxy, C₁-C₇-alkoxy-C₁-C₇-alkoxy, carboxy-C₁-C₇-alkoxy, amino-C₁-C₇-alkoxy, N—C₁-C₇-alkanoylamino-C₁-C₇-alkoxy, carbamoyl-C₁-C₇-alkyl, carbamoyl-C₁-C₇-alkoxy, N—C₁-C₇-alkylcarbamoyl-C₁-C₇-alkoxy, C₁-C₇alkanoyl, C₁-C₇-alkyloxy-C₁-C₇-alkanoyl, C₁-C₇-alkoxy-C₁-C₇-alkanoyl, carboxyl, carbamoyl and N—C₁-C₇-alkoxy-C₁-C₇-alkylcarbamoyl, more preferably C₁-C₇-alkyl, halo, hydroxy-C₁-C₇-alkyl, C₁-C₇-alkoxy-C₁-C₇-alkyl, C₁-C₇-alkanoylamino-C₁-C₇-alkyl, C₁-C₇-alkoxy-C₁-C₇alkoxy, carbamoyl-C₁-C₇-alkyl, N—C₁-C₇-alkylcarbamoyl-C₁-C₇-alkyl, N—C₁-C₇-haloalkylcarbamoyl-C₁-C₇-alkyl, in particular methyl, pentyl, methoxy-propyl, methoxy-butyl, ethoxy-ethyl, hydroxy-butyl, methoxypropyloxy, F, CH₃—C(O)—NH—CH₂CH₂, NH₂—CO—CH₂CH₂CH₂, N(CH₂CH₃)—CO—CH₂, N(CH₂CF₃)—CO—CH₂. The heterocyclyl moiety is preferably substituted on the N if present. Most preferably, the heterocyclyl is unsubstituted.

In a second embodiment R⁵ is preferably unsubstituted or substituted heterocyclyl.

The heterocyclyl moiety preferably mono- or bicyclic, more preferably bicyclic. Preferred are aromatic ring systems, or partially saturated ring systems, in particular whereby one of the rings is aromatic and the other is saturated or partially saturated, most preferred are saturated. The heterocyclyl moiety has preferably 1, 2 or 3, more preferably 1 or 2, most preferably 2, heteroatoms selected from O, N or S, more preferably O or N. The ring system contains preferably an oxo moiety. Particularly preferred examples include 5- to 10-membered mono- or bicyclic heterocyclyl, such as bicyclic 9 or 10-membered rings preferably containing a nitrogen atom, in particular, quinolyl, isoquinolyl, 1,2,3,4-tetrahydro-1,4-benzoxazinyl, 2H-1,4-benzoxazin-3(4H)-only, 3,4-dihydro-1H-quinolin-2-onyl, or 4H-benzo[1,4]thiazin-3-onyl; indolyl, 1H-indazolyl, benzothiophenyl, imidazo[1,2-a]pyridyl or 3H-benzooxazol-2-only; or 5- or 6-membered monocyclic rings containing an O or N atom such as tetrahydrofuranyl, tetrahydropyranyl, furanyl, pyranyl, piperidinyl, pyrrolidinyl, imidazolyl, triazolyl, piperazinyl, morpholinyl, pyrimidinyl or pyridinyl, where each heterocyclyl is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkylsulfonylamino-C₁-C₇-alkyl, carboxy-C₁-C₇-alkyl, C₁-C₇-alkoxycarbonyl-C₁-C₇-alkyl, halo, hydroxy, C₁-C₇-alkoxy, C₁-C₇-alkoxy-C₁-C₇-alkoxy, carboxy-C₁-C₇-alkoxy, amino-C₁-C₇-alkoxy, N—C₁-C₇-alkanoylamino-C₁-C₇-alkoxy, carbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carboxyl, carbamoyl and N—C₁-C₇-alkoxy-C₁-C₇-alkylcarbamoyl, more preferably C₁-C₇-alkyl, halo, hydroxy-C₁-C₇-alkyl, C₁-C₇-alkoxy-C₁-C₇-alkyl, C₁-C₇-alkanoylamino-C₁-C₇-alkyl, C₁-C₇-alkoxy-C₁-C₇-alkoxy, carbamoyl-C₁-C₇-alkyl, N—C₁-C₇-alkylcarbamoyl-C₁-C₇-alkyl, N—C₁-C₇-haloalkylcarbamoyl-C₁-C₇-alkyl, in particular methyl, pentyl, methoxy-propyl, methoxy-butyl, ethoxy-ethyl, hydroxy-butyl, methoxypropyloxy, F, CH₃—C(O)—NH—CH₂CH₂, NH₂—CO—CH₂CH₂CH₂, N(CH₂CH₃)—CO—CH₂, N(CH₂CF₃)—CO—CH₂. The heterocyclyl moiety is preferably substituted on the N if present. Most preferably, the heterocyclyl is unsubstituted.

In a third embodiment R⁵ is preferably unsubstituted or substituted aryl.

Preferred examples of aryl include phenyl or naphthyl, more preferably phenyl. When the aryl moiety is substituted, it is preferably mono- or di-substituted. Most preferably aryl is di-substituted. Suitable substituents are as defined herein, preferably C₁-C₇-alkyl, —O—C₁-C₇-alkyl, halo-C₁-C₇-alkyl, —O-halo-C₁-C₇-alkyl, halo, hydroxy, nitro, amino, amino-C₁-C₇-alkyl, carboxy 1, cyano, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy-C₁-C₇-alkoxy, C₁-C₇-alkanoyloxy-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, C₁-C₇-alkylsulfonylamino-C₁-C₇-alkyl, carboxy-C₁-C₇-alkyl, C₁-C₇-alkoxycarbonyl-C₁-C₇-alkyl, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkyl, N—C₁-C₇-alkylcarbamoyl-C₁-C₇-alkyl, N—C₁-C₇-haloalkylcarbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carbamoyl and N—C₁-C₇-alkoxy-C₁-C₇-alkylcarbamoyl, more preferably C₁-C₇-alkyl, —O—C₁-C₇-alkyl, halo-C₁-C₇-alkyl, halo, cyano, hydroxy-C₁-C₇-alkyl, C₁-C₇alkoxy-C₁-C₇-alkoxy, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, in particular, methyl, O-methyl, Cl, Br, CN, methoxypropyloxy, N(methoxypropyl)-amino, N(acetyl)-amino, and N(methoxypropyl)(acetyl)-amino.

In a fourth embodiment R5 is preferably unsubstituted or substituted cycloalkyl.

Preferred examples of cycloalkyl include C₃-C₁₀-cycloalkyl, more preferably C₃-C₇-Cycloalkyl, still more preferably C₃-, C₄-, C₅- or C₆-cycloalkyl. When the cycloalkyl moiety is substituted, it is preferably mono- or di-substituted. Most preferably cycloalkyl is unsubstituted. Suitable substituents are as defined herein, preferably C₁-C₇-alkyl, —O—C₁-C₇-alkyl, halo-C₁-C₇-alkyl, —O-halo-C₁-C₇-alkyl, halo, hydroxy, nitro, amino, amino-C₁-C₇-alkyl, carboxyl, cyano, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy-C₁-C₇-alkoxy, C₁-C₇-alkanoyloxy-C₁-C₇-alkyl, C₁-C₇-alkoxy-C₁-C₇-alkylamino-C₁-C₇-alkyl, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, C₁-C₇-alkylsulfonylamino-C₁-C₇-alkyl, carboxy-C₁-C₇-alkyl, C₁-C₇-alkoxycarbonyl-C₁-C₇-alkyl, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkyl, N—C₁-C₇-alkylcarbamoyl-C₁-C₇-alkyl, N—C₁-C₇-haloalkylcarbamoyl-C₁-C₇-alkyl, carbamoyl-C₁-C₇-alkoxy, N—C₁-C₇-alkylcarbamoyl-C₁-C₇-alkoxy, C₁-C₇-alkanoyl, C₁-C₇-alkyloxy-C₁-C₇-alkanoyl, C₁-C₇-alkoxy-C₁-C₇-alkanoyl, carbamoyl and N—C₁-C₇-alkoxy-C₁-C₇-alkylcarbamoyl, more preferably C₁-C₇-alkyl, —O—C₁-C₇-alkyl, halo-C₁-C₇-alkyl, halo, cyano, hydroxy-C₁-C₇-alkyl, C₁-C₇-alkoxy-C₁-C₇-alkoxy, C₁-C₇-alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, in particular, methyl, O-methyl, Cl, Br, CN, methoxypropyloxy, N(methoxypropyl)-amino, N(acetyl)-amino, and N(methoxypropyl)(acetyl)-amino.

The first and second embodiment are particularly preferred.

In a preferred embodiment, Y is —(C═O)— and R⁵ is unsubstituted or substituted alkyl as defined herein, preferably benzyl or CH₂-tetrahydropyranyl.

In a preferred embodiment, Y is —(C═O)— and R⁵ is unsubstituted or substituted heterocyclyl as defined herein, preferably unsubstituted or substituted tetrahydropyranyl or pyrazinyl.

In a preferred embodiment, Y is —(C═O)O— and R⁵ is unsubstituted or substituted heterocyclyl as defined herein, preferably tetrahydropyranyl.

### Preferred Definitions for X

In a preferred embodiment, X is CH₂.

In a preferred embodiment, X is O.

### Preferred Definitions for Ar

Ar is preferably unsubstituted or substituted aryl or unsubstituted or substituted mono- or bicyclic aromatic heterocyclyl, whereby suitable substituents are selected from a substitutent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of each other, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, or is phenyl, naphthyl, phenyl- or naphthyl-C₁-C₇-alkyl and halo-C₁-C₇-alkyl; where said substituent —(C₀-C₇-alkylene)-(X)ᵣ—(C₁-C₇-alkylene)-(Y)ₛ—(C₀-C₇-alkylene)-His preferably C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇alkyl, C₁-C₇-alkanoyloxy-C₁-C₇-alkyl, amino-C₁-C₇-alkyl, such as aminomethyl, (N—) mono- or (N,N-) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇alkoxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇alkanoylamino, C₁-C₇-alkylsulfonylamino, C₁-C₇-alkoxy-carbonyl, halo-C₁-C₇-alkoxycarbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl and N-mono- or N,N-di-(C₁-C₇-alkyl)-aminosulfonyl;

more preferably, Ar is phenyl, naphthyl, indolyl, benzimidazolyl, benzofuranyl, quinolinyl, preferably phenyl or indolyl, wherein each is unsubstituted or substituted by one or more, e.g. up to three, substitutents selected from the group consisting of
a substitutent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of each other, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, or is phenyl, naphthyl, phenyl- or naphthyl-C₁-C₇-alkyl and halo-C₁-C₇-alkyl; where said substituent —(C₀-C₇-alkylene)-(X)ᵣ—(C₁-C₇-alkylene)-(Y)ₛ—(C₀-C₇-alkylene)-His preferably C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, such as aminomethyl, (N—) mono- or (N,N-) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇alkoxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkoxy-carbonyl, halo-C₁-C₇-alkoxycarbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl and N-mono- or N,N-di-(C₁-C₇-alkyl)-aminosulfonyl.

In a first embodiment, Ar is unsubstituted or substituted aryl.

Preferred examples for the aryl moiety are phenyl and naphthyl, more preferably phenyl. When the aryl moiety is substituted, it is preferably mono- or di-substituted. Naphthyl is preferably mono-substituted and phenyl is preferably mono- or di-substituted, more preferably di-substituted. Suitable substituents for the aryl moiety are as defined herein:
preferably a substitutent of the formula —(C₀-C₇-alkylene)-(X)_{X}—(C₁-C₇-alkylene)-(Y)ₛ—(C₀-C₇-alkylene)-H where CO-alkylene means that a bond is present instead of bound alkylene, r and s, each independently of the other, are 0 or 1 and each of X and Y, if present and independently of each other, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, or is phenyl, naphthyl, phenyl- or naphthyl-C₁-C₇-alkyl and halo-C₁-C₇-alkyl; where said substituent —(C₀-C₇-alkylene)-(X)_{X}—(C₁-C₇-alkylene)-(Y)S—(C₀-C₇-alkylene)-His preferably C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, such as aminomethyl, (N—) mono- or (N,N-) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇alkoxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkoxy-carbonyl, halo-C₁-C₇-alkoxycarbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl or N-mono- or N,N-di-(C₁-C₇-alkyl)-aminosulfonyl; more preferably, —(C₀-C₇-alkylene)-(X)ᵣ—(C₁-C₇-alkylene)-(Y)ₛ—(C₀-C₇-alkylene)-H, wherein r and s are 0 or 1 and Y and X are independently O, NH or NH—CO—O—, halo-C₁-C₇-alkyl, halo, hydroxy, phenyl- or naphthyloxy, phenyl- or naphthyl-C₁-C₇-alkyloxy, nitro, amino, amino-C₁-C₇-alkyl, carboxyl, and cyano. Preferred examples of —(C₀-C₇-alkylene)-(X)ᵣ—(C₁-C₇-alkylene)-(Y)ₛ—(C₀-C₇-alkylene)-H include —(O or NH)—C₁-C₇-alkyl, —C₁-C₇-alkyl, —(O or NH)—C₁-C₇-alkylene-(O or NH)—C₁-C₇-alkyl, —(O or NH)—C₁-C₇-alkylene-(O or NH)—H, —C₁-C₇-alkylene-(O or NH)—C₁-C₇-alkylene-(O or NH)—C₁-C₇-alkyl, —C₁-C₇-alkylene-(O or NH)—C₁-C₇-alkyl, or —C₁-C₇-alkylene-NH—CO—O—C₁-C₇-alkyl, most preferably —OMe, —OC₃H₆OMe, —NH-butyl, methyl, ethyl, —C₂H₄—NH—CO-OMe, —CH₂OC₂H₄OMe, —OC₂H₄OC₂H₅, —OC₃H₆OH, —C₂H₄OMe, —C₃H₆OMe and —NH—C₃H₆OMe. Most preferably the aryl moiety is unsubstituted or substituted with OMe and/or —OC₃H₆OMe.

In a second embodiment, Ar is unsubstituted or substituted mono- or bicyclic aromatic heterocyclyl.

The heterocyclyl moiety has preferably 1, 2 or 3, more preferably 1 or 2 heteroatoms selected from O, N or S, more preferably O or N. Particularly preferred examples include pyrrolyl, furanyl, thienyl, pyridyl, pyrimidinyl, indolyl, benzimidazolyl, benzopyrazolyl, benzofuranyl, quinolinyl, more preferably indolyl, benzimidazolyl, benzofuranyl, quinolinyl, most preferably indolyl. When the heterocyclyl moiety is substituted, it is preferably mono-substituted. Suitable substituents for the heterocyclyl moiety are as defined herein, preferably —(C₀-C₇-alkylene)-(X)ᵣ—(C₁-C₇-alkylene)-(Y)ₛ—(C₀-C₇-alkylene)-H, wherein r and s are 0 or 1 and Y and X are independently O, NH or NH—CO—O—, halo-C₁-C₇-alkyl, halo, hydroxy, phenyl- or naphthyloxy, phenyl- or naphthyl-C₁-C₇-alkyloxy, nitro, amino, amino-C₁-C₇-alkyl, carboxyl, and cyano. Preferred examples of —(C₀-C₇-alkylene)-(X)ᵣ(C₁-C₇-alkylene)-(Y)ₛ—(C₀-C₇-alkylene)-H include —O or NH)—C₁-C₇-alkyl, —C₁-C₇-alkyl, —(O or NH)—C₁-C₇-alkylene-(O or NH)—C₁-C₇-alkyl, —(O or NH)—C₁-C₇-alkylene-(g or NH)—H, —C₁-C₇-alkylene-(O or NH)—C₁-C₇-alkylene-(O or NH)—C₁-C₇-alkyl, —C₁-C₇-alkylene-(O or NH)—C₁-C₇-alkyl, or —C₁-C₇-alkylene-NH—CO—O—C₁-C₇-alkyl, more preferably —OMe, —OC₂H₄OMe, —NH-butyl, methyl, ethyl, —C₂H₄—NH—CO-OMe, —CH₂OC₂H₄OMe, —OC₂H₄OC₂H₅, —OC₃H₆OH, —C₂H₄OMe, —C₃H₆OMe and —NH—C₃H₆OMe, yet more preferably —NH-propyl, —C₂H₄OMe and —C₃H₆OMe. Most preferably the heterocyclyl moiety is unsubstituted or substituted by Me, —C₂H₄OMe or —C₃H₆OMe.

Particularly preferred for Ar is the moiety

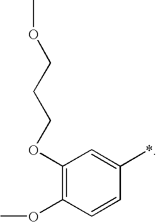

Particular embodiments of the invention, especially of compounds of the formula I and/or salts thereof, are provided in the Examples—the invention thus, in a very preferred embodiment, relates to a compound of the formula I, or a salt thereof, selected from the compounds given in the Examples, as well as their use.

### Process of Manufacture

A compound of formula I, or a salt thereof, is prepared analogously to methods that, for other compounds, are in principle known in the art, so that for the novel compounds of the formula I the process is novel at least as analogy process, especially as described or in analogy to methods described herein in the illustrative Examples, or modifications thereof, preferably in general by

A) reacting an acid of the formula II

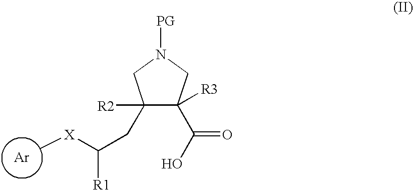

or a reactive derivative thereof, wherein R¹, R², R³, X, and Ar are as defined herein for a compound of the formula I and PG is a protecting group, with either
(i) an amino compound of the formula III,

R4(R5Y)RNH (III)

wherein R⁴, R⁵ and Y are as defined herein for a compound of the formula I, under condensation conditions and
reducing the carbonyl group in the resulting compound of the formula IV

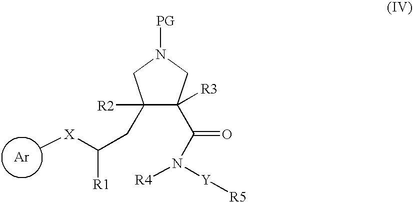

wherein R¹, R², R³, R⁴, R⁵, X, Y, Ar and PG are as defined for compounds of formulae II and III, to a methylene group, and, to obtain, upon removal of the protecting group PG, a compound of the formula I wherein R¹, R², R³, R⁴, R⁵, X, Y and Ar are as defined herein;
or
(ii) with an amino compound of the formula V,

R⁴—NH₂ (V)

wherein R⁴ is as defined herein for a compound of the formula I, to give a compound of the formula VI,

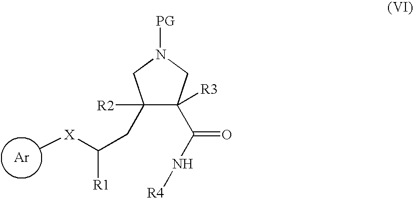

wherein R¹, R², R³, R⁴, X and Ar are as defined herein for a compound for the formula I and PG is a protecting group, and
reducing the carbonyl group whereby a compound of the formula VII

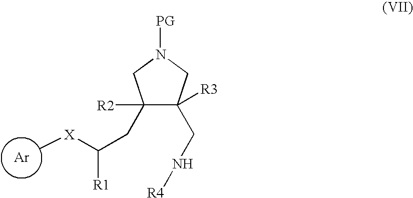

is obtained wherein R¹, R², R³, R⁴, X, Ar and PG are as defined for a compound of the formula VI, and reacting the compound of the formula VII with a compound of the formula VIII,

R5-Y-Z (VIII)

wherein R⁵ and Y are as defined herein for a compound of the formula I and Z is a leaving group, to obtain, upon removal of the protecting group PG, a compound of the formula I wherein R¹, R², R³, R⁴, R⁵, X, Y and Ar are as defined herein; or
B) reacting an aldehyde of the formula IX,

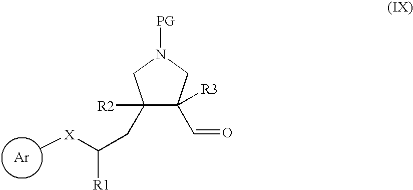

wherein R¹, R², R³, X and Ar are as defined herein for a compound of the formula I and PG is a protecting group, either
(i) with an amino compound of the formula III as defined above under the conditions for reductive amination and, to obtain, upon removal of the protecting group PG, a compound of formula I wherein R¹, R², R³, R⁴, R⁵, X, Y and Ar are as defined herein;
or
(ii) with an amino compound of the formula V as defined above whereby a compound of the formula VII

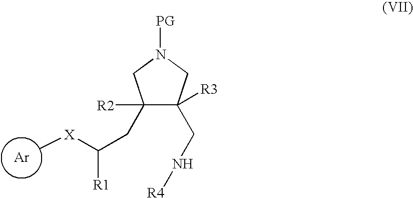

is obtained, wherein R¹, R², R³, R⁴, X, and Ar are as defined for a compound of the formula I herein and PG is a protecting group, under conditions of reductive amination and then reacting the compound of the formula (VII) with a compound of the formula VIII as defined above, to obtain, upon removal of the protecting group PG, a compound of formula I wherein R¹, R², R³, R⁴, R⁵, X, Y and Ar are as defined herein; or
C) oxidizing a compound of the formula X,

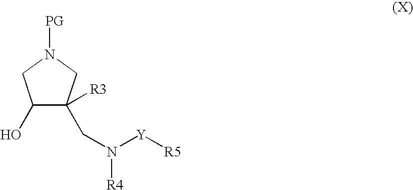

wherein R³, R⁴, R⁵, and Y are as just defined, PG is a protecting group to obtain a compound of formula XI

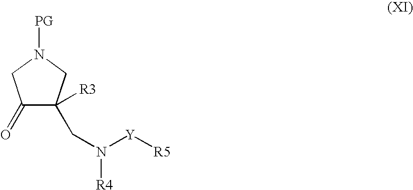

wherein R³, R⁴, R⁵, Y and PG are as just defined;
reacting the compound of formula XI with a metallo reagent of the formula XII,

Ar—X—CHR¹—CH₂—Mg-Hal (XII)

wherein R¹, Ar and X as just defined and Hal is halo, to obtain, upon removal of the protecting group PG, a corresponding compound of the formula I, wherein R² is hydroxyl and R¹, R³, R⁴, R⁵, X, Y and Ar are as defined herein;
and, if desired, subsequent to any one or more of the processes mentioned under (A) to (C) converting an obtainable compound of the formula I or a protected form thereof into a different compound of the formula I, converting a salt of an obtainable compound of formula I into the free compound or a different salt, converting an obtainable free compound of formula I into a salt thereof, and/or separating an obtainable mixture of isomers of a compound of formula I into individual isomers;
where in any of the starting materials, in addition to specific protecting groups PG, further protecting groups may be present, and any protecting groups are removed at an appropriate stage in order to obtain the corresponding compound of the formula I, or a salt thereof.

### Preferred Reaction Conditions

The preferred reaction conditions for the reactions mentioned above under A) to C), as well as for the transformations and conversions, are as follows:

The condensation reaction in A) (i) between an acid of the formula II, or a reactive derivative thereof, and an amino compound of the formula III preferably takes place under customary condensation conditions, where among the possible reactive derivatives of an acid of the formula II reactive esters (such as the hydroxybenzotriazole (HOBT), pentafluorophenyl, 4-nitrophenyl or N-hydroxysuccinimide ester), acid halogenides (such as the acid chloride or bromide) or reactive anhydrides (such as mixed anhydrides with lower alkanoic acids or symmetric anhydrides) are preferred. Reactive carbonic acid derivatives can also be formed in situ. The reaction is carried out by dissolving the compounds of formulae II and III in a suitable solvent, for example a halogenated hydrocarbon, such as methylene chloride, N,N-dimethylformamide, N,N-dimethylacetamide, N-methyl-2-pyrrolidone, methylene chloride, or a mixture of two or more such solvents, and by the addition of a suitable base, for example triethylamine or diisopropylethylamine (DIEA) and, if the reactive derivative of the acid of the formula II is formed in situ, a suitable coupling agent that forms a preferred reactive derivative of the carbonic acid of formula III in situ, for example dicyclohexylcarbodiimide/1-hydroxybenzotriazole (DCC/HOBT); bis(2-oxo-3-oxazolidinyl)phosphinic chloride (BOPCl); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); (benzotriazol-1-yloxy)-tripyrrolidinophosphonium-hexafluorophosphate (PyBOP) or 1-(3-dimethylaminopropyl)-3-ethylcarbodiimide hydrochloride/hydroxybenzotriazole (EDCVHOBT). For review of some other possible coupling agents, see e.g. Klauser; Bodansky, Synthesis 1972, 453-463. The reaction mixture is preferably stirred at a temperature of between approximately −20 and 50° C., especially between 0° C. and 30° C., e.g. at room temperature. The reaction is preferably carried out under an inert gas, e.g. nitrogen or argon.

The removal of a protecting group under A) (i), e.g. PG, such as tert-butoxycarbonyl, benzyl or 2-(trimethylsilyl)-ethoxycarbonyl, takes place under standard conditions, see also the literature mentioned below under General Process Conditions. For example, tert-butoxycarbonyl is removed in the presence of an acid, e.g. a TFA or hydrohalic acid, such as HCl, in an appropriate solvent, e.g. an ether, such as dioxane, at customary temperatures, e.g. at room temperature, the removal of benzyl can be achieved e.g. by reaction with ethylchloroformate or 2-trimethylsilylethyl-chloroformate in an appropriate solvent, e.g. toluene, at elevated temperatures, e.g. from 80 to 110° C., and subsequent removal of the resulting ethoxycarbonyl group by hydrolysis in the presence of a base, e.g. an alkali metal hydroxide, such as potassium hydroxide, in an appropriate solvent, e.g. in an alcohol, such as ethanol, at elevated temperatures, e.g. from 80 to 120° C., and the removal of 2-(trimethylsilyl)-ethoxycarbonyl can be achieved, for example, by reaction with a tetra-lower alkylammonium fluoride, such as tetraethylammoniumfluoride, in an appropriate solvent or solvent mixture, e.g. a halogenated hydrocarbon, such as methylene chloride, and/or a nitrile, such as acetonitrile, preferably at elevated temperatures, e.g. under reflux conditions.

The reduction of a carbonyl group can preferably take place in the presence of an appropriate complex hydride, e.g. borane dimethylsulfide complex, in an appropriate solvent, such as an ether, e.g. tetrahydrofurane, at preferred temperatures between room temperature and the reflux temperature of the reaction mixture or at 140-150° C.

The removal of (a) protecting group(s) can be achieved before or after the reduction of a carbonyl group.

In step A) (ii), the reaction between a compound of the formula V with an acid of the formula II, or a reactive derivative thereof, and the subsequent reduction of the carbonyl group preferably takes place under conditions analogous to those described above for reaction A) (i). The reaction between a compound of the formula VII and a compound of the formula VIII under A) (ii) preferably takes place under customary substitution conditions, e.g. in the case where an aryl moiety R5 is to be coupled and Z is halo, e.g. iodo or bromo, in the presence of copper (e.g. Venus copper), sodium or potassium iodide and a base, such as potassium carbonate, in the presence or preferably absence of an appropriate solvent, e.g. at elevated temperatures in the range from, for example, 150 to 250° C., or (especially if Z in formula VIII is bromo) in the presence of a strong base, such as an alkali metal alcoholate, e.g. sodium tert-butylate, in the presence of an appropriate catalyst, such as [Pd(μ-Br)(t-Bu₃P)]₂, in the presence of an appropriate solvent, e.g. an aromatic solvent, such as toluene, at preferred temperatures between room temperature and the reflux temperature of the mixture, or (e.g. where the moiety R5 is unsubstituted or substituted alkyl) in the presence of a base, such as an alkali metal carbonate, such as potassium carbonate, if useful in the presence of an alkali metal halogenide, e.g. sodium or potassium iodide, in an appropriate solvent, such as dimethyl formamide, at preferably elevated temperatures, e.g. between 50° C. and the reflux temperature of the mixture, or, where R5 is to be bound via a carbonyl or sulfonyl group, under condensation conditions e.g. as described above under A) (i); the reactions can preferably take place under a protective gas, such as nitrogen or argon. The subsequent removal of (a) protecting group(s) takes place as described above under A) (i).

The reaction under B) (i) between an aldehyde compound of the formula IX with an amino compound of the formula III preferably takes place under customary conditions for reductive amination, e.g. in the presence of an appropriate reducing (e.g. hydrogenation) agent, such as hydrogen in the presence of a catalyst or a complex hydride, e.g. sodium triacetoxyborohydride or sodium cyanoborohydride, in an appropriate solvent, such as a halogenated hydrocarbon, e.g. methylene chloride or 1,2,-dichloroethane, and optionally a carbonic acid, e.g. acetic acid, at preferred temperatures between −10° C. and 50° C., e.g. from 0° C. to room temperature; the subsequent removal of protecting groups takes place e.g. as described above under A) (i).

The reaction under B) (ii) between an aldehyde compound of the formula IX with an amino compound of the formula V takes place under customary conditions for reductive amination, e.g. as just described under B) (i), the subsequent reaction under B) (ii) between the resulting compound of the formula VII and a compound of the formula VIII under customary substitution conditions, e.g. as described above for reaction A) (ii) and the removing of (a) protecting group(s) takes place e.g. as described above under A) (i).

The oxidation under C) of a hydroxy compound of the formula X to a corresponding oxo compound of the formula XI preferably takes place in the presence of an appropriate oxidant, such as Dess-Martin-periodinane, in an appropriate solvent, e.g. a halogenated hydrocarbon, e.g. methylene chloride, at preferred temperatures from 0° C. to 50° C., e.g. at room temperature. The optional subsequent conversion of an oxo group into a thioxo group (═S) can take place in the presence of Lawesson's reagent or under under customary thionation conditions, the conversion of oxo into an (unsubstituted or substituted) imino by reaction with protected ammonia (for unsubstituted imino) or a primary amine corresponding to a substituted imino to be introduced under customary Schiff base formation conditions. Removal of protecting groups takes place preferably as described under A) (i).

The coupling under C) between a metallo reagent of the formula XII and a compound of the formula XI takes place under customary reaction conditions, e.g. under Grignard coupling conditions, in an appropriate solvent, e.g. an ether, such as diethyl ether, at preferred temperatures in the range from −100 to −50° C., e.g. at −80 to −70° C. Removal of protecting groups takes place preferably as described under A) (i) (a).

### Optional Reactions and Conversions

Compounds of the formula I, or protected forms thereof directly obtained according to any one of the preceding procedures or after introducing protecting groups anew, which are included subsequently as starting materials for conversions as well even if not mentioned specifically, can be converted into different compounds of the formula I according to known procedures, where required after removal of protecting groups.

For example, a lower alkoxy (especially methoxy) group present as a substituent of an aryl moiety in a compound of the formula I (e.g. as part of R¹) can be converted into the corresponding hydroxy substituent by reaction, e.g., with boron tribromide in an appropriate solvent, e.g. a halogen ated hydrocarbon, at preferred temperatures in the range from −100 to −50° C., e.g. at −80 to −70° C., yielding the corresponding hydroxy compound of the formula I.

A cyano group present as substituent on a compound of the formula I can be converted into an aminomethyl group e.g. by hydrogenation in the presence of a catalyst, such as a transition metal catalyst, e.g. Raney-Nickel, under customary conditions, e.g. in an alcohol, such as methanol, at preferred temperatures between 0° C. and 50° C., e.g. at room temperature, to yield the corresponding amino compound of the formula I, yielding a corresponding compound of the formula I.

An amino group present as a substituent on a compound of the formula I can be converted into an acyl(especially lower-alkanoyl)-amino group e.g. by acylation with a carbonic or sulfonic acid, or a reactive derivative thereof, e.g. the corresponding acid halogenide, such as the acid chloride, or under in situ formation of the corresponding active derivative, under conditions analogous to those described above under A) (i), yielding the corresponding acyl-amino compound of the formula I.

An amino group present as a substituent on a compound of the formula I can be converted into an N,N-di-(C₁-C₇-alkyl)- or N,N-di-(phenyl- or naphthyl-C₁-C₇-alkyl)-amino group by alkylation e.g. with a corresponding N,N-di-(C₁-C₇-alkyl)- or N,N-di-(phenyl- or naphthyl-C₁-C₇-alkyl)-halogenide, e.g. -chloride or -bromide, or by reductive amination with a corresponding oxo compound (wherein one of the methylene groups in the C₁-C₇-alkyl-comprising compound used as precursor carries oxo instead of two hydrogen atoms) under conditions of reductive amination, e.g. analogous to those described under process variant B) (i) described above, yielding a corresponding compound of the formula I.

A nitro group present as substituent on a compound of the formula I can be converted into an amino group e.g. by hydrogenation in the presence of a catalyst, such as a transition metal catalyst, e.g. Raney-Nickel, under customary conditions, e.g. in an alcohol, such as methanol, at preferred temperatures between 0° C. and 50° C., e.g. at room temperature, to yield the corresponding amino compound of the formula I, yielding a corresponding compound of the formula I.

A hydroxy group present as a substituent in a compound of the formula I can be converted into an alkylated or acylated hydroxy group, e.g. C₁-C₇-alkoxy-C₁-C₇-alkoxy, C₁-C₇-alkoxy or phenyl- or naphthyl-C₁-C₇-alkyloxy, by reaction with a corresponding alkylhalogenide or acyl-halogenide, e.g. a C₁-C₇-alkoxy-C₁-C₇-alkylchloride or -bromide, a C₁-C₇-alkylchloride or -bromide or a phenyl- or naphthyl-C₁-C₇-alkyl-chloride or -bromide, under appropriate customary substitution reaction conditions, e.g. in the presence of a base, such as an alkali metal carbonate, e.g. potassium carbonate, or a strong base, such as an alkali metal hydride, e.g. sodium hydride, in an appropriate solvent, e.g. an amide, such as dimethylformamide, at preferred temperatures from 0 to 100° C., e.g. from room temperature to 80° C., yielding a corresponding compound of the formula I.

An imino group in a compound of the formula I, e.g. —NH— as part of a substituent in a compound of the formula I comprising an N-heterocyclic moiety, can be transformed into a C₁-C₇-alkoxy-C₁-C₇-alkylimino group by reaction with a C₁-C₇-alkoxy-C₁-C₇-alkylhalogenide, e.g. chloride or bromide, under reaction conditions as described in the directly preceding paragraph, yielding a corresponding compound of the formula I.

An amino group in a compound of the formula I can be converted into an unsubstituted or substituted alkylamino (e.g. C₁-C₇-alkylamino, such as isopropylamino), unsubstituted or substituted cycloalkylamino (e.g. cyclohexylamino), unsubstituted or substituted aryl-alkyl-amino, unsubstituted or substituted heterocyclyl-alkylamino, unsubstituted or substituted cycloalkyl-alkylamino, alkyloxycarbonylamino, alkylcarbonylamino, substituted or unsubstituted alkylsulfonylamino, substituted or unsubstituted arylsulfonylamino (such as C₁-C₇-alkyl-phenylsulfonyl, e.g. tosyl), substituted or unsubstituted heterocyclylsulfonylamino or substituted or unsubstituted cycloalkylsulfonylamino by reaction with the corresponding unsubstituted or substituted alkane, unsubstituted or substituted cycloalkane, unsubstituted or substituted aryl-alkane, unsubstituted or substituted heterocyclyl-alkane, unsubstituted or substituted cycloalkyl-alkane carrying a keto group instead of a methylene or a formyl group instead of a methyl in the alkyl part, under customary reaction conditions for reductive amination, e.g. as described above under B) (i); or by reaction with a substituted or unsubstituted alylsulfonylhalogenide, substituted or unsubstituted arylsulfonylhalogenide, substituted or unsubstituted heterocyclylsulfonylhalogenide or substituted or unsubstituted cycloalkylsulfonylhalogenide under customary reaction conditions, e.g. in the presence of a tertiary amine, such as triethylamine, in an appropriate solvent, e.g. a halogenated hydrocarbon, such as methylene chloride, at preferred temperatures from 0° C. to 50° C., e.g. at room temperature; yielding a corresponding compound of the formula I.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent. Internal salts of compounds of formula I containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Stereoisomeric mixtures, e.g. mixtures of diastereomers, can be separated into their corresponding isomers in a manner known per se by means of appropriate separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral acid, or by chromatography, for example by HPLC, using chromatographic substrates with chiral ligands.

Intermediates and final products can be worked up and/or purified according to customary methods, e.g. using chromatographic methods, distribution methods, (re-) crystallization, and the like.

### Starting Materials

Starting Materials, including intermediates, for compounds of the formula I, can be prepared, for example, according to methods that are known in the art, according to methods described in the examples or methods analogous to those described in the examples, and/or they are known or commercially available.

In the subsequent description of starting materials and intermediates and their synthesis, R¹, R², R³, R⁴, R⁵, X, Y, Ar and PG have the meanings given above or in the Examples for the respective starting materials or intermediates, if not indicated otherwise directly or by the context. Protecting groups, if not specifically mentioned, can be introduced and removed at appropriate steps in order to prevent functional groups, the reaction of which is not desired in the corresponding reaction step or steps, employing protecting groups, methods for their introduction and their removal are as described above or below, e.g. in the references mentioned under “General Process Conditions”.

A compound of the formula II can, for example, be obtained by reacting a compound of the formula XIV,

PG-NH—CH₂—CHR3-CN (XIV)

wherein PG is a protecting group, especially benzyl, with a compound of the formula XV,

Ar—X—CHR1-CH═CR2-CH₂-Hal (XV)

wherein Hal is halo, such as bromo, or a different leaving group, such as tosyl, in the presence of a base, such as an alkali metal hydroxide, e.g. NaOH, and e.g. benzyl-tri-(N-butyl)ammonium bromide, in an appropriate solvent, e.g. a halogenated hydrocarbon, such as methylene chloride, and/or water, preferably at a temperature from 10 to 50° C., e.g. 40° C., treating the resulting compound of the formula XVI

Ar—X—CHR1-CH═CR2-CH₂—N(PG)-CH₂—CHR3-CN (XVI)

wherein the substituents have the meanings just described in the presence of a strong base, such as sodium hydride, in an appropriate solvent, e.g. hexamethylphosphoroamide, at preferred temperatures between −10 and 40° C., thus obtaining a compound of the formula XVII,

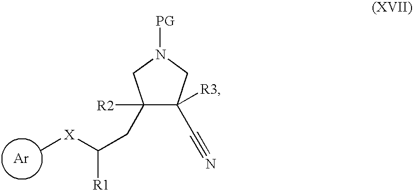

which is then hydrolyzed, e.g. in the presence of a hydrohalic acid, such as HCl, in an appropriate solvent, e.g. acetic acid, water or a mixture thereof, at elevated temperatures, e.g. under reflux, to the corresponding compound of the formula II.

A starting material of the formula II can also be obtained by reacting a compound of the formula XVIII,

Ar—X—CHR1-CH₂—CHO (XVIII)

with a compound of the formula XIX,

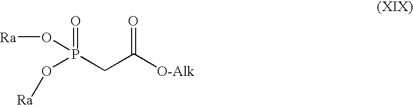

wherein Ra is ethyl or 2,2,2-trifluoroethyl and Alk is lower alkyl, in the presence of a strong base, e.g. sodium hydride e.g. in tetrahydrofurane at preferred temperatures in the range from −10 to 40° C., or in the presence of potassium hexamethyldisiliazane and a crown ether, e.g. 18-crown-6, e.g. in tetrahydrofurane and/or toluene at low temperatures, e.g. from −90 to −70° C., to give a compound of the formula XX,

Ar—X—CHR1-CH₂—CH═CH—COOAlk (XX)

which compound is then reacted with a compound of the formula XXI,

(H₃C)₃Si—CH₂—N(PG)-CH₂—O—CH₃ (XXI)

wherein PG is a protecting group as defined e.g. for a compound of the formula II, in the presence of an acid, e.g. trifluoroacetic acid, in an appropriate solvent, e.g. toluene, at preferred temperatures between −10 and 40° C., to give a compound of the formula XXII,

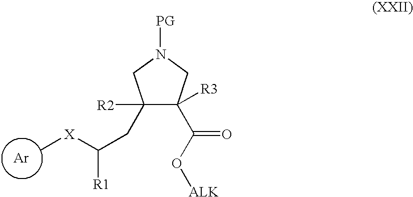

(if desired, the protecting group PG may be replaced by a different protecting group, e.g. benzyl by tert-butoxycarbonyl), and then hydrolysis to remove the Alk-group to give the corresponding free acid of the formula II or reduction, e.g. with lithium aluminium chloride in tetrahydrofurane and followed by oxidation under Dess-Martin-conditions to the corresponding aldehyde of the formula IX which can thus also be obtained.

A corresponding corn pound of the formula IX can be obtained by reducing the carboxy function in a compound of the formula II as obtained in the preceding paragraph, e.g. in the presence of borane dimethylsulfide complex in e.g. tetrahydrofurane at from −20° C. to 40° C., to the corresponding hydroxymethyl function and oxidation of this to the corresponding formyl function, e.g. with Dess-Martin periodinane e.g. in wet methylenechloride at temperatures from 0 to 50° C.

In all formulae above where present, the central pyrrolidine and its substituents at positions 3 and 4 may be present in any one or more of the following configurations, and/or mixtures of the corresponding isomers may be formed and/or separated into the individual isomers at appropriate stages:

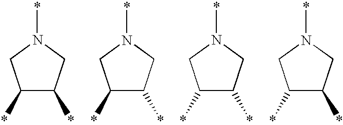

wherein the left lower bond is also on the left side in any of the formulae intermediates or starting materials as shown above or final products of the formula I, the right lower bond on the right side.

### General Process Conditions

The following applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula I is designated a “protecting group”, unless the context indicates otherwise. The protection of functional groups by such protecting groups, the protectting groups themselves, and the reactions appropriate for their introduction and removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to 60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula I described as being preferred. Special preference is given to reaction conditions that are identical or analogous to those mentioned in the Examples.

### Pharmaceutical Use, Pharmaceutical Preparations and Methods

As described above, the compounds of the present invention are inhibitors of renin activity and, thus, may be employed for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders, and the like.

The present invention further provides pharmaceutical compositions comprising a therapeutically effective amount of a pharmacologically active compound of the instant invention, alone or in combination with one or more pharmaceutically acceptable carriers.

The pharmaceutical compositions according to the present invention are those suitable for enteral, such as oral or rectal, transdermal and parenteral administration to mammals, including man, to inhibit renin activity, and for the treatment of conditions associated with (especially inappropriate) renin activity. Such conditions include hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders and the like.

Thus, the pharmacologically active compounds of the invention may be employed in the manufacture of pharmaceutical compositions comprising an effective amount thereof in conjunction or admixture with excipients or carriers suitable for either enteral or parenteral administration. Preferred are tablets and gelatin capsules comprising the active ingredient together with:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethylleneglycol; for tablets also
c) binders, e.g., magnesium aluminum silicate, starch paste, gelatin, tragacanth, methylcellulose, sodium carboxymethylcellulose and or polyvinylpyrrolidone; if desired
d) disintegrants, e.g., starches, agar, alginic acid or its sodium salt, or effervescent mixtures; and/or
e) absorbents, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and pre-determ ined rate over a prolonged period of time, and means to secure the device to the skin.

Accordingly, the present invention provides pharmaceutical compositions as described above for the treatment of conditions mediated by renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders, as well as methods of their use.

The pharmaceutical compositions may contain a therapeutically effective amount of a compound of the formula I as defined herein, either alone or in a combination with another therapeutic agent, e.g., each at an effective therapeutic dose as reported in the art. Such therapeutic agents include:

a) antidiabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; peroxisome proliferator-activated receptor (PPAR) ligands; protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as LAF237;
b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) anti-obesity agents such as orlistat; and
d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibittors; ACE/NEP inhibitors such as omapatrilat, sampatrilat and fasidotril; angiotensin II antagonists such as candesartan, eprosartan, irbesartan, losartan, telmisartan and valsartan, in particular valsartan; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amLodipine, bepridil, diltiazem, felodipine, nicardipine, nimodipine, nifedipine, nisoldipine and verapamil; aldosterone receptor antagonists; and aldosterone synthase inhibitors.

Other specific anti-diabetic compounds are described by Patel Mona in Expert Opin Investig Drugs, 2003, 12(4), 623-633, in the FIGS. 1 to 7, which are herein incorporated by reference. A compound of the present invention may be administered either simultaneously, before or after the other active ingredient, either separately by the same or different route of administration or together in the same pharmaceutical formulation.

The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical compositions comprising a therapeutically effective amount of a compound of the invention alone or in combination with a therapeutically effective amount of another therapeutic agent, preferably selected from antidiabetics, hypolipidemic agents, anti-obesity agents or anti-hypertensive agents, most preferably from antidiabetics, anti-hypertensive agents or hypolipidemic agents as described above.

The present invention further relates to pharmaceutical compositions as described above for use as a medicament.

The present invention further relates to use of pharmaceutical compositions or combinations as described above for the preparation of a medicament for the treatment of conditions mediated by (especially inappropriate) renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders, and the like.

Thus, the present invention also relates to a compound of formula I for use as a medicament, to the use of a compound of formula I for the preparation of a pharmaceutical composition for the prevention and/or treatment of conditions mediated by (especially inappropriate) renin activity, and to a pharmaceutical composition for use in conditions mediated by (especially inappropriate) renin activity comprising a compound of formula I, or a pharmaceutically acceptable salt thereof, in association with a pharmaceutically acceptable diluent or carrier material therefore.

The present invention further provides a method for the prevention and/or treatment of conditions mediated by (especially inappropriate) renin activity, which comprises administering a therapeutically effective amount of a compound of the present invention to a warm-blooded animal, especially a human, in need of such treatment.

A unit dosage for a mammal of about 50-70 kg may contain between about 1 mg and 1000 mg, advantageously between about 5-600 mg of the active ingredient. The therapeutically effective dosage of active compound is dependent on the species of warm-blooded animal (especially mammal, more especially human), the body weight, age and individual condition, on the form of administration, and on the compound involved.

In accordance with the foregoing the present invention also provides a therapeutic combination, e.g., a kit, kit of parts, e.g., for use in any method as defined herein, comprising a compound of formula I, or a pharmaceutically acceptable salt thereof, to be used concomitantly or in sequence with at least one pharmaceutical composition comprising at least another therapeutic agent, preferably selected from anti-diabetic agents, hypolipidemic agents, anti-obesity agents or anti-hypertensive agents. The kit may comprise instructions for its administration.

Similarly, the present invention provides a kit of parts comprising: (i) a pharmaceutical composition comprising a compound of the formula I according to the invention; and (ii) a pharmaceutical composition comprising a compound selected from an anti-diabetic, a hypolipidemic agent, an anti-obesity agent, an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound of formula I, or a pharmaceutically acceptable salt thereof, and at least a second drug substance, said second drug substance preferably being an anti-diabetic, a hypolipidemic agent, an anti-obesity agent or an anti-hypertensive agent, e.g., as indicated above.

Preferably, a compound of the invention is administered to a mammal in need thereof.

Preferably, a compound of the invention is used for the treatment of a disease which responds to a modulation of (especially inappropriate) renin activity.

Preferably, the condition associated with (especially inappropriate) renin activity is selected from hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

Finally, the present invention provides a method or use which comprises administering a compound of formula I in combination with a therapeutically effective amount of an anti-diabetic agent, a hypolipidemic agent, an anti-obesity agent or an anti-hypertensive agent.

Ultimately, the present invention provides a method or use which comprises administering a compound of formula I in the form of a pharmaceutical composition as described herein.

The above-cited properties are demonstrable in vitro and in vivo tests using advantageously mammals, e.g., mice, rats, rabbits, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The concentration level in vitro may range between about 10⁻³ molar and 10⁻¹⁰ molar concentrations. A therapeutically effective amount in vivo may range depending on the route of administration, between about 0.001 and 500 mg/kg, preferably between about 0.1 and 100 mg/kg.

As described above, the compounds of the present invention have enzyme-inhibiting properties. In particular, they inhibit the action of the natural enzyme renin. Renin passes from the kidneys into the blood where it effects the cleavage of angiotensinogen, releasing the decapeptide angiotensin I which is then cleaved in the lungs, the kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume which increase can be attributed to the action of angiotensin II. Inhibitors of the enzymatic activity of renin lead to a reduction in the formation of angiotensin 1, and consequently a smaller amount of angiotensin II is produced. The reduced concentration of that active peptide hormone is a direct cause of the hypotensive effect of renin inhibitors.

The action of renin inhibitors may be demonstrated inter alia experimentally by means of in vitro tests, the reduction in the formation of angiotensin I being measured in various systems (human plasma, purified human renin together with synthetic or natural renin substrate).

Inter alia the following in vitro tests may be used:

Recombinant human renin (expressed in Chinese Hamster Ovary cells and purified using standard methods) at 7.5 nM concentration is incubated with test compound at various concentrations for 1 h at RT in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile_His_Thr-Lys(DABCYL)-Arg9 is added to a final concentration of 2 μM and increase in fluorescence is recorded at an excitation wave-length of 350 nm and at an emission wave-length of 500 nm in a microplate spectro-fluorimeter. IC50 values are calculated from percentage of inhibition of renin activity as a function of test compound concentration (Fluorescence Resonance Energy Transfer, FRET, assay). Compounds of the formula I, in this assay, preferably show IC₅₀ values in the range from 10 nM to 20 μM

Alternatively, recombinant human renin (expressed in Chinese Hamster Ovary cells and purified using standard methods) at 0.5 nM concentration is incubated with test compound at various concentrations for 2 h at 37° C. in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile_His_Thr-Lys(DABCYL)-Arg9 is added to a final concentration of 4 μM and increase in fluorescence is recorded at an excitation wave-length of 340 nm and at an emission wave-length of 485 nm in a microplate spectro-fluorimeter. IC₅₀ values are calculated from percentage of inhibition of renin activity as a function of test compound concentration (Fluorescence Resonance Energy Transfer, FRET, assay). Compounds of the formula I, in this assay, preferably show IC₅₀ values in the range from 10 nM to 20 μM.

In another assay, human plasma spiked with recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC50 values are calculated from percentage of inhibition of renin activity as a function of test compound concentration. Compounds of the formula I, in this assay, preferably show IC₅₀ values in the range from 10 nM to 20 μM.

In another assay, recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC50 values are calculated from percentage of inhibition of renin activity as a function of test compound concentration. Compounds of the formula I, in this assay, preferably show IC₅₀ values in the range from 10 nM to 20 μM.

In animals deficient in salt, renin inhibitors bring about a reduction in blood pressure. Human renin may differ from the renin of other species. In order to test inhibitors of human renin, primates, e.g., marmosets (Callithrix jacchus) may be used, because human renin and primate renin are substantially homologous in the enzymatically active region. Inter alia the following in vivo tests may be used:

Compounds can be tested in vivo in primates as described in the literature (see for example by Schnell C R et al. Measurement of blood pressure and heart rate by telemetry in conscious, unrestrained marmosets. Am J Physiol 264 (Heart Circ Physiol 33). 1993: 1509-1516; or Schnell C R et al. Measurement of blood pressure, heart rate, body temperature, ECG and activity by telemetry in conscious, unrestrained marmosets. Proceedings of the fifth FELASA symposium: Welfare and Science. Eds BRIGHTON. 1993.

The following Examples, while representing preferred embodiments of the invention, serve to illustrate the invention without limiting its scope.

### Abbreviations:

AcOH acetic acid
DIBAL-H diisobutylaluminum hydride
4-DMAP 4-dimethylamino-pyridine
DMF dimethylformamide
DMPU 1,3-dimethyl-3,4,5,6-tetrahydro-2(1H)-pyrimidinone
DMSO dimethylsulfoxide
DPPA diphenylphosphoryl azide
EDCl 1-(3-dimethylaminopropyl)-3-ethylcarbodiimide hydrochloride
EtOAc ethyl acetate
Et₃N triethylamine
EtOH ethanol
Flow flow rate
h hour(s)
HMPA hexamethylphosphoroamide
HOBt 1-hydroxybenzotriazole

### HPLC High Performance Liquid Chromatography

iPrOH isopropanol
L liter(s)
KHMDS potassium hexamethyldisilazane

### LC-MS Liquid Chromatography/Mass Spectrometry

LDA lithium diisopropylamine
Me methyl
MeI methyl iodide
MeOH methanol
MesCl methanesulfonyl chloride
Min minute(s)
mL milliliter

### MS Mass Spectrometry

NMM 4-methylmorpholine

### NMR Nuclear Magnetic Resonance

Pd/C palladium on charcoal
RT room temperature
TBAF tetra-butylammonium fluoride
TBDMS-Cl tert-butyldimethylsilyl chloride
TBDMS tert-butyldimethylsilyl
TBME tert-Butylmethylether
TFA trifluoroacetic acid
THF tetrahydrofurane
RP reversed phase

### TLC Thin Layer Chromatography

tᵣ retention time

### Trademarks

Celite=Celite® (The Celite Corporation)=filtering aid based on diatomaceous earth
Nucleosil=Nucleosil®, trademark of Machery & Nagel, Diuren, FRG for HPLC materials

Temperatures are measured in degrees Celsius. Unless otherwise indicated, the reactions take place at RT.

TLC conditions: R_{f} values for TLC are measured on 5×10 cm TLC plates, silica gel F₂₅₄, Merck, Darmstadt, Germany.

The general procedure to produce compounds of formula I is exemplified in Schemes 1 to 3 below and as described in more detail in the examples.

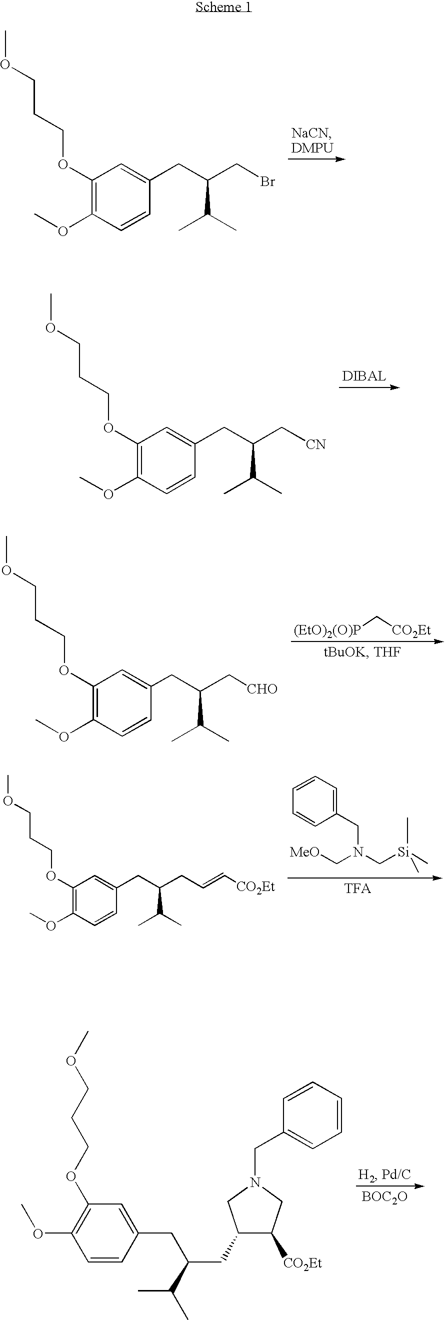

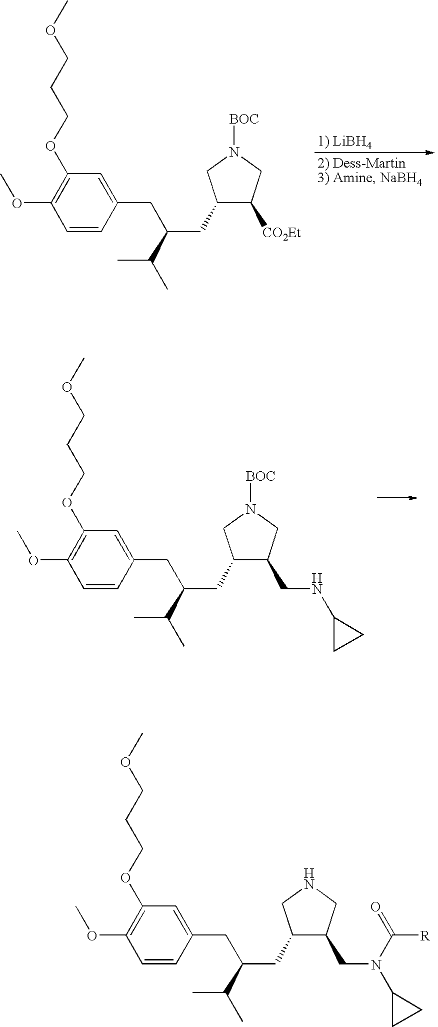

### Example 1

### N-Cyclopropyl-N-((3S,4S)-4-{(R)-2-[4-methoxy-3-(3-methoxy-propoxy)-benzyl]-3-methyl-butyl}-pyrrolidin-3-ylmethyl)-2-phenyl-acetamide

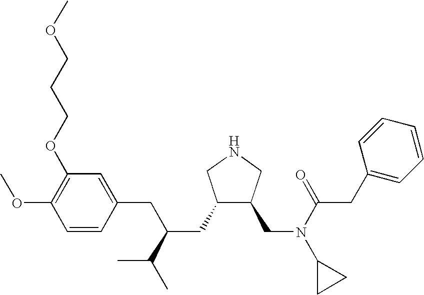

To (3R,4S)-3-[(cyclopropyl-phenylacetyl-amino)-methyl]-4-{(R)-2-[4-methoxy-3-(3-methoxy-propoxy)-benzyl]-3-methyl-butyl}-pyrrolidine-1-carboxylic acid tert-butyl ester (250 mg, 0.393 mmol) is added a 4M HCl solution in dioxane (0.98 mL, 3.90 mmol), and stirring is continued at room temperature overnight. The mixture is then freeze-dried in high vacuo overnight to give the title compound as its mono hydrochloride salt. RP-HPLC: t_{R}=5.28 min (Nucleosil C18-HD column, 10-100% CH₃CN/H₂O/5 min, 100% CH₃CN/3 min, CH₃CN and H₂O containing 0.1% TFA, flow: 1.5 mL/min; column: 4×70 mm; particle size 3 μm). MS: 537.4 [M+H]⁺.

The starting materials are prepared according to Scheme 1 as follows:

### A. (S)-3-[4-Methoxy-3-(3-methoxy-propoxy)-benzyl]-4-methyl-pentanenitrile

To a solution of 4-((R)-2-bromomethyl-3-methyl-butyl)-1-methoxy-2-(3-methoxy-propoxy)-benzene, prepared as described in Helv. Chimica Acta 2003, 86, 2848-2870, (30.5 g, 84.9 mmol) in DMPU (450 mL) is added in portions NaCN (17.5 g, 357 mmol) with stirring. The reaction mixture is warmed to 50° C. for 2 h, followed by addition of water after cooling to ambient temperature. The aqueous layer is extracted with EtOAc, and the combined organics are repeatedly washed with water, dried (Na₂SO₄) and concentrated. The crude product is purified by flash chromatography on silica gel (eluent gradient:hexane/EtOAc 85:15 to 70:30) to give the title compound as colorless oil. TLC, R_{f} (hexane/EtOAc 3:1)=0.32. MS: 306.2 [M+H]⁺ and 323.2 [M+18]⁺.

### B. (S)-3-[4-Methoxy-3-(3-methoxy-propoxy)-benzyl]-4-methyl-pentanal

To a solution of (S)-3-[4-methoxy-3-(3-methoxy-propoxy)-benzyl]-4-methyl-pentanenitrile (12.2 g, 39.9 mmol) in toluene (20 mL), cooled to −60° C., is added dropwise with stirring a 1.7 M solution of DIBAL-H (32.9 mL, 55.9 mmol). After 15 min at −60° C., the mixture is slowly warmed to ambient temperature with stirring overnight. The mixture is cooled to 0° C., followed by dropwise addition of EtOAc (23 mL). Stirring is continued for 1 h at room temperature, the mixture is again cooled to 0° C., followed by dropwise addition of a saturated aqueous solution of NH₄Cl (108 mL) and, after one additional hour, by addition of 2M H₂SO₄ (108 mL) and diethyl ether (100 mL). After warming to room temperature over 1 h, the layers are separated and the aqueous phase is extracted with diethyl ether. The combined organics are washed with saturated NaHCO₃ and water, dried (Na₂SO₄) and concentrated. The crude product is purified by flash chromatography on silica gel (hexane/EtOAc 3:1) to give the title compound. TLC, R_{f} (hexane/EtOAc 3:1)=0.35. MS: 326.2 [M+18]⁺.

### C. (R)-5-[4-Methoxy-3-(3-methoxy-propoxy)-benzyl]-6-methyl-hept-2-enoic acid ethyl ester

A solution of triethyl 2-phosphonoacetate (7.41 g, 33.1 mmol) in THF (20 mL) is added dropwise over 5 min to a solution potassium tert-butoxide (3.09 g, 27.5 mmol) in THF (40 mL) under an argon atmosphere. After stirring for 30 min at room temperature, a solution of (S)-3-[4-methoxy-3-(3-methoxy-propoxy)-benzyl]-4-methyl-pentanal (7.08 g, 13.8 mmol) in THF (20 mL) is added dropwise and stirring is continued for 30 min. The mixture is then poured into a diluted aqueous NH₄Cl solution and the water phase is extracted with diethyl ether. The combined organics are washed with saturated aqueous NH₄Cl, dried (Na₂SO₄) and concentrated. The crude material is purified by flash chromatography on silica gel (hexane/EtOAc 8:2) to give the title compound as colorless oil. TLC, R_{f} (hexane/EtOAc 3:1) 0.36. MS: 396.2 [M+18]⁺.

### D. (3S*,4S*)-1-Benzyl-4-{(R)-2-[4-methoxy-3-(3-methoxy-propoxy)-benzyl]-3-methyl-butyl}-pyrrolidine-3-carboxylic acid ethyl ester

To a solution of (R)-5-[4-methoxy-3-(3-methoxy-propoxy)-benzyl]6-methyl-hept-2-enoic acid ethyl ester (5.13 g, 12.6 mmol) in toluene (50 mL), cooled to 0° C., is subsequently added under an argon atmosphere N-methoxy-N-(trimethylsilylmethyl)benzylamine (3.82 g, 15.1 mmol; Lancaster 19412) and a solution of trifluoroacetic acid (0.095 mL, 1.26 mmol) in CH₂Cl₂ (0.5 mL) in a dropwise fashion. Stirring is continued at 0° C. for 30 min and at room temperature overnight. To the reaction mixture is then added a saturated aqueous NaHCO₃ solution, and the water layer is extracted with EtOAc, the combined organics are dried over Na₂SO₄, filtered and concentrated. The residue is purified by flash chromatography (eluent gradient:hexane/EtOAc 3:1 to 2:1) to give the title compound as a mixture of trans-configured diastereomers. Colorless oil. TLC, R_{f} (hexane/EtOAc 3:1)=0.24. MS: 512.2 [M+H]⁺.

### E. (3S*,4S*)-4(R)-2-[4-Methoxy-3-(3-methoxy-propoxy)-benzyl]-3-methyl-butyl)-pyrrolidine-1,3-dicarboxylic acid 1-tert-butyl ester 3-ethyl ester

A solution of (3S*,4S*)-1-benzyl-4{(R)-2-[4-methoxy-3-(3-methoxy-propoxy)-benzyl]-3-methyl-butyl}-pyrrolidine-3-carboxylic acid ethyl ester (5.05 g, 9.87 mmol) and di-tert-butyl dicarbonate (2.59 g, 11.8 mmol) in analytical grade EtOH (100 mL) is hydrogenated for 18 h in the presence of catalytic 10% Pd/C (0.5 g; Engelhard 4505) at 25° C. under atmospheric pressure. The reaction mixture is filtered through Celite®, and the combined filtrated are concentrated. Purification by flash chromatography (hexane/EtOAc 3:1) gives the title compound as mixture of trans-configured diastereomers. Colorless oil. TLC, R_{f} (hexane/EtOAc 3:1)=0.31. MS: 522.1 [M+H]⁺; 539.1 [M+18]⁺.

### F. (3S*,4S*)-3-Hydroxymethyl-4-{(R)-2-[4-methoxy-3-(3-methoxy-propoxy)-benzyl]-3-methyl-butyl}-pyrrolidine-1-carboxylic acid tert-butyl ester

To a solution of (3S*,4S*)-4-{(R)-2-[4-methoxy-3-(3-methoxy-propoxy)-benzyl]-3-methyl-butyl}-pyrrolidine-1,3-dicarboxylic acid 1-tert-butyl ester 3-ethyl ester (3.83 g, 7.34 mmol) in THF (50 mL), cooled to 0 to 5° C., is added dropwise with stirring a solution of LiBH₄ (0.160 g, 7.34 mmol) in THF (20 mL). The reaction mixture is warmed to room temperature for 3 h, and then heated to 60° C. overnight. After cooling to ambient temperature, 2M NaOH (50 mL) is added, and the water phase is extracted with diethyl ether, the combined organics are dried (Na₂SO₄), filtered and concentrated in vacuo to give the title compound as a ca. 1:1 mixture of trans-configured diastereomers. Colorless oil. TLC, R_{f} (CH₂Cl₂/MeOH 95:5)=0.45. t_{R} (HPLC, Nucleosil C18-HD, 5-100% CH₃CN/H₂O/6 min, 100% CH₃CN/1.5 min, CH₃CN and H₂O containing 0.1% TFA, flow: 1.0 mL/min): 5.93 min. MS: 480.3 [M+H]⁺; 497.4 [M+18]⁺.

### G. (3S,4S)- and (3R,4R)-3-Hydroxymethyl-4-{(R)-2-[4-methoxy-3-(3-methoxy-propoxy)-benzyl]-3-methyl-butyl}-pyrrolidine-1-carboxylic acid tert-butyl ester

Chromatographic separation of the ca. 1:1 mixture of the trans-diastereomers (3S*,4S*)-3-hydroxymethyl-4{(R)-2-[4-methoxy-3-(3-methoxy-propoxy)-benzyl]-3-methyl-butyl}-pyrrolidine-1-carboxylic acid tert-butyl ester (0.8 g per run; material dissolved in EtOH (5 mL) and n-hexane (50 mL)) is performed on a Chiralcel OD® column (particle size: 20 μm; column dimensions: 5×50 cm) using n-hexane/EtOH/MeOH (96:2:2) as eluent (flow rate: 120 mL/min; detection: UV 210 nm). Pure fractions containing the single diastereomers from several chromatography runs are combined, solvents are evaporated and the residues are dried in high vacuo to afford the title compounds. The first eluting diastereomer corresponds to (3R,4R)-3-hydroxymethyl-4-{(R)-2-[4-methoxy-3-(3-methoxy-propoxy)-benzyl]-3-methyl-butyl}-pyrrolidine-1-carboxylic acid tert-butyl ester. Colorless oil. Diastereomeric purity de >99.8% by analytical chiral HPLC (Chiralcel OD-HO (1157); column 0.46×25 cm; solvent:n-hexane/EtOH/MeOH 94:3:3; flow rate 1 mL/min; detection UV 210 nm) with t_{R}=9.59 min. RP-HPLC: t_{R}=5.87 min (C18 column, 5-100% CH₃CN/H₂O/6 min, 100% CH₃CN/1.5 min, CH₃CN and H₂O containing 0.1% TFA, flow: 1.0 mL/min).

The second eluting diastereomer corresponds to the title compound (3S,4S)-3-hydroxy methyl-4-{(R)-2-[4-methoxy-3-(3-methoxy-propoxy)-benzyl]-3-methyl-butyl}pyrrolidine-1-carboxylic acid tert-butyl ester. Colorless oil. Diastereomeric purity de>99.8% by analytical chiral HPLC with t_{R}=11.7 min. RP-HPLC: t_{R}=5.89 min (Nucleosil C18-HD, 5-100% CH₃CN/H₂O/6 min, 100% CH₃CN/1.5 min, CH₃CN and H₂O containing 0.1% TFA, flow: 1.0 mL/min).

### H. (3S,4S)-3-Formyl-4-{(R)-2-[4-methoxy-3-(3-methoxy-propoxy)-benzyl]-3-methyl-butyl}-pyrrolidine-1-carboxylic acid tert-butyl ester

To a solution of (3S,4S)-3-hydroxymethyl-4{(R)-2-[4-methoxy-3-(3-methoxy-propoxy)-benzyl]-3-methyl-butyl}-pyrrolidine-1-carboxylic acid tert-butyl ester (0.70 g, 1.46 mmol) and Dess-Martin periodinane (0.74 g, 1.75 mmol; RareChem AR PA 0035) in absolute CH₂Cl₂ (10 mL) is dropwise added wet CH₂Cl₂ (29 μL of water in 30 mL of CH₂Cl₂) with rigorous stirring. After stirring the reaction mixture at room temperature overnight, another aliquot of Dess-Martin periodinane (0.42 g, 1.05 mmol) is added in two portions and stirring is continued for 20 h, followed by evaporation of the solvent in vacuo to a small volume. The residue is taken up in diethyl ether (50 mL), and the organic layer is washed with a 1:1 (v/v) mixture of a 10% aqueous Na₂S₂O₃ solution and a saturated aqueous NaHCO₃ solution (50 mL). The organic layer is dried (Na₂SO₄), filtered and concentrated to give the title compound as crude product. Colorless oil. RP-HPLC: t_{R}=6.14 min (Nucleosil C18-HD, 5-100% CH₃CN/H₂O/6 min, 100% CH₃CN/1.5 min, CH₃CN and H₂O containing 0.1% TFA, flow: 1.0 mL/min). MS: 478.2 [M+H]⁺.

### I. (3R,4S)-3-Cyclopropylaminomethyl-4-{(R)-2-[4-methoxy-3-(3-methoxy-propoxy)-benzyl]-3-methyl-butyl}-pyrrolidine-1-carboxylic acid tert-butyl ester

To a solution of (3S,4S)-3-formyl-4{(R)-2-[4-methoxy-3-(3-methoxy-propoxy)-benzyl]-3-methyl-butyl}-pyrrolidine-1-carboxylic acid tert-butyl ester (0.72 g, 1.51 mmol) in MeOH (10 mL) containing 2% of acetic acid is added cyclopropylamine (0.53 mL, 7.54 mmol). After stirring for 1 h at room temperature, NaBH₄ (0.11 g, 3.01 mmol) is added in portions and stirring is continued for 1 h. The reaction mixture is concentrated in vacuo to a small volume, followed by addition of a concentrated aqueous NaHCO₃ solution. The water phase is extracted with EtOAc, the combined organics are dried (Na₂SO₄) and evaporated in vacuo to give the title compound as crude product. Colorless oil. RP-HPLC: t_{R}=5.42 min (Nucleosil C18-HD, 5-100% CH₃CN/H₂O/6 min, 100% CH₃CN/1.5 min, CH₃CN and H₂O containing 0.1% TFA, flow: 1.0 mL/min). MS: 519.4 [M+H]⁺.

### J. (3R,4S)-3-[(Cyclopropyl-phenylacetyl-amino)-methyl]-4-{(R)-2-[4-methoxy-3-(3-methoxy-Propoxy)-benzyl]-3-methyl-butyl}-pyrrolidine-1-carboxylic acid tert-butyl ester

A mixture of (3R,4S)-3-cyclopropylaminomethyl-4-{(R)-2-[4-methoxy-3-(3-methoxy-propoxy)-benzyl]-3-methyl-butyl}-pyrrolidine-1-carboxylic acid tert-butyl ester (250 mg, 0.40 mmol), phenylacetic acid (65 mg, 0.48 mmol), 1-hydroxy-benzotriazol hydrate (65 mg, 0.48 mmol), N-(3-dimethylaminopropyl)-N′-ethyl-carbodiimide hydrochloride (92 mg, 0.48 mmol) and triethylamine (67 μL, 0.48 mmol) in CH₂Cl₂ (7.0 mL) is stirred at room temperature overnight. The reaction mixture is then diluted with CH₂Cl₂, and the organic layer is subsequently washed with 1M HCl (5 mL), saturated NaHCO₃ and brine, dried over Na₂SO₄ and evaporated. The residue is purified by flash chromatography on silica gel (eluent gradient:hexane/EtOAc 3:1 to 1:1) to give the title compound as colorless oil. RP-HPLC: t_{R}=6.73 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 637.4 [M+H]⁺.

### Example 2

### N-Cyclopropyl-N-((3R,4R)-4{(R)-2-[4-methoxy-3-(3-methoxy-propoxy)-benzyl]-3-methyl-butyl}-pyrrolidin-3-ylmethyl)-2-phenyl-acetamide

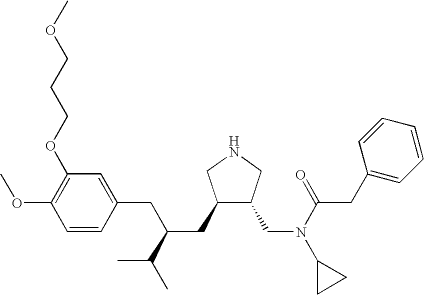

The title compound is prepared by the procedure described in Example 1, starting from (3S,4R)-3-[(cyclopropyl-phenylacetyl-amino)-methyl]-4-{(R)-2-[4-methoxy-3-(3-methoxy-propoxy)-benzyl]-3-methyl-butyl}pyrrolidine-1-carboxylic acid tert-butyl ester (220 mg, 0.345 mmol) and 4M HCl in dioxane (0.86 mL, 3.45 mmol). After freeze-drying in high vacuo overnight the title compound is obtained as its mono hydrochloride salt. RP-HPLC: t_{R}=5.33 min (Nucleosil C18-HD column, 10-100% CH₃CN/H₂O/5 min, 100% CH₃CN/3 min, CH₃CN and H₂O containing 0.1% TFA, flow: 1.5 mL/min; column: 4×70 mm; particle size 3 μm). MS: 537.4 [M+H]⁺.

The starting materials are prepared as follows:

### A. (3R,4R)-3-Formyl-4-{(R)-2-[4-methoxy-3-(3-methoxy-propoxy)-benzyl]-3-methyl-butyl}-pyrrolidine-1-carboxylic acid tert-butyl ester

From (3R,4R)-3-hydroxymethyl-4-{(R)-2-[4-methoxy-3-(3-methoxy-propoxy)-benzyl]-3-methyl-butyl}-pyrrolidine-1-carboxylic acid tert-butyl ester (0.71 g, 1.48 mmol) and Dess-Martin periodinane (1.18 g, 2.80 mmol) in CH₂Cl₂ by the procedure as described in Example 1, reaction step H. The title compound is obtained as colorless oil. RP-HPLC: t_{R}=6.15 min (Nucleosil C18-HD, 5-100% CH₃CN/H₂O/6 min, 100% CH₃CN/1.5 min, CH₃CN and H₂O containing 0.1% TFA, flow: 1.0 mL/min).

### B. (3S,4R)-3-Cyclopropylaminomethyl-4-{(R)-2-[4-methoxy-3-(3-methoxy-propoxy)-benzyl]-3-methyl-butyl}-pyrrolidine-1-carboxylic acid tert-butyl ester

From (3R,4R)-3-formyl-4-{(R)-2-[4-methoxy-3-(3-methoxy-propoxy)-benzyl]-3-methyl-butyl}-pyrrolidine-1-carboxylic acid tert-butyl ester (0.75 g, 1.57 mmol), cyclopropylamine (0.55 mL, 7.85 mmol) and NaBH₄ (0.119 g, 3.14 mmol) by the procedure as described in Example 1, reaction step 1. The title compound is obtained as colorless oil. RP-HPLC: t_{R}=5.34 min (Nucleosil C18-HD, 5-100% CH₃CN/H₂O/6 min, 100% CH₃CN/1.5 min, CH₃CN and H₂O containing 0.1% TFA, flow: 1.0 mL/min). MS: 519.4 [M+H]⁺.

### C. (3S,4R)-3-[(Cyclopropyl-phenylacetyl-amino)-methyl]-4-{(R)-2-[4-methoxy-3-(3-methoxy-propoxy)-benzyl]-3-methyl-butyl}-pyrrolidine-1-carboxylic acid tert-butyl ester

As described for Example 1, reaction step J, from (3S,4R)-3-cyclopropylaminomethyl-4-{(R)-2-[4-methoxy-3-(3-methoxy-propoxy)-benzyl]-3-methyl-butyl}-pyrrolidine-1-carboxylic acid tert-butyl ester (0.25 g, 0.40 mmol), phenylacetic acid (65 mg, 0.48 mmol), 1-hydroxy-benzotriazol hydrate (65 mg, 0.48 mmol), N-(3-dimethylaminopropyl)-N′-ethyl-carbodiimide hydrochloride (92 mg, 0.48 mmol) and triethylamine (67 μL, 0.48 mmol) in CH₂Cl₂ (7.0 mL). The title compound is obtained as colorless oil. RP-HPLC: t_{R}=6.72 min (Nucleosil C18-HD, 5-100% CH₃CN/H₂O/6 min, 100% CH₃CN/1.5 min, CH₃CN and H₂O containing 0.1% TFA, flow: 1.0 mL/min). MS: 637.4 [M+H]⁺.

### Example 3

### (S)-N-Cyclopropyl-2-hydroxy-N-((3S,4S)-4-{(R)-2-[4-methoxy-3-(3-methoxy-propoxy)-benzyl]-3-methyl-butyl}-pyrrolidin-3-ylmethyl)-3-methyl-butyramide

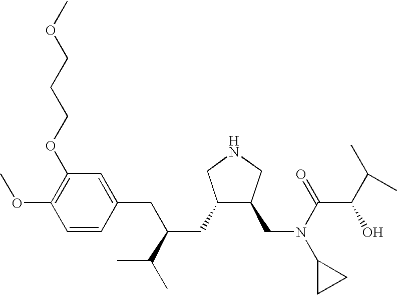

The title compound is prepared analogously as described for the title compound Example 1, starting from (3R,4S)-3-{[cyclopropyl-((S)-2-hydroxy-3-methyl-butyryl)-amino]-methyl}-4-{(R)-2-[4-methoxy-3-(3-methoxy-propoxy)-benzyl]-3-methyl-butyl}-pyrrolidine-1-carboxylic acid tert-butyl ester (193 mg, 0.312 mmol) and N—BOC deprotection in 4M HCl in dioxane (0.78 mL, 3.1 mmol) at room temperature overnight. The title compound is obtained after freeze-drying as the mono hydrochloride salt. White solid. TLC, R_{f} (CH₂Cl₂/MeOH/10% NH₃ 9:1)=0.31. RP-HPLC: t_{R}=4.92 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 519.3 [M+H]⁺.

The starting materials are prepared as follows:

### A. (3R,4S)-3-{[Cyclopropyl((S)-2-hydroxy-3-methyl-butyryl)-amino]-methyl}-4-{(R)-2-[4-methoxy-3-(3-methoxy-propoxy)-benzyl]-3-methyl-butyl}-pyrrolidine-1-carboxylic acid tert-butyl ester

A mixture of (3R,4S)-3-cyclopropylaminomethyl-4-(R)-2-[4-methoxy-3-(3-methoxy-propoxy)-benzyl]-3-methyl-butyl)pyrrolidine-1-carboxylic acid tert-butyl ester (140 mg, 0.25 mmol), (S)-2-hydroxy-3-methylbutyric acid (35 mg, 0.30 mmol), 1-hydroxy-7-azabenzotriazol (40 mg, 0.30 mmol), N-(3-dimethylaminopropyl)-N′-ethyl-carbodiimide hydrochloride (85 mg, 0.44 mmol) and triethylamine (41 μL, 0.30 mmol) in CH₂Cl₂ (7.0 mL) is stirred at room temperature overnight. The reaction mixture is then diluted with CH₂Cl₂, and the organic layer is subsequently washed with 1M HCl (5 mL), saturated NaHCO₃ and brine, dried over Na₂SO₄ and evaporated. The residue is purified by flash chromatography on silica gel (eluent gradient:hexane/EtOAc 3:1 to 1:1) to give the title compound as colorless oil. TLC, R_{f} (hexane/EtOAc 1:1)=0.42. RP-HPLC: t_{R}=6.46 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 619.4 [M+H]⁺.

### Example 4

### Cyclopropyl-((3S,4S)-4-{(R)-2-[4-methoxy-3-(3-methoxy-propoxy)-benzyl]-3-methyl-butyl}-pyrrolidin-3-ylmethyl)-carbamic acid tetrahydropyran-4-yl ester

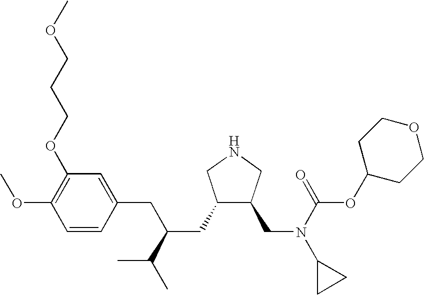

The title compound is prepared analogously as described for the title compound Example 1, starting from (3S,4S)-3-{[cyclopropyl-(tetrahydropyran-4-yloxycarbonyl)-amino]-methyl}-4-{(R)-2-[4-methoxy-3-(3-methoxy-propoxy)-benzyl]-3-methyl-butyl}-pyrrolidine-1-carboxylic acid tert-butyl ester (110 mg, 0.170 mmol) and N—BOC deprotection in 4M HCl in dioxane (0.43 mL, 1.70 mmol) at room temperature overnight. The title compound is obtained after freeze-drying as the mono hydrochloride salt. TLC, R. (CH₂Cl₂/MeOH/10% NH₃ 9:1)=0.46. RP-HPLC: t_{R}=5.00 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 547.4 [M+H]⁺.

The starting materials are prepared as follows (Scheme 2):

### A. (3S,4S)-3-{[Cyclopropyl-(tetrahydropyran-4-yloxylcarbonyl)-amino]-methyl)}-4-{(R)-2-[4-methoxy-3-(3-methoxy-propoxy)-benzyl]-3-methyl-butyl}-pyrrolidine-1-carboxylic acid tert-butyl ester

To a solution of tetrahydro-4-pyranol (45 mg, 0.44 mmol; Aldrich 19, 823-4) in CH₂Cl₂ (10 mL) is subsequently added triphosgene (48 mg, 0.16 mmol) and 4-DMAP (172 mg, 1.41 mmol) and the reaction mixture is stirred for 3 h at room temperature. Then a solution of (3R,4S)-3-cyclopropylaminomethyl-4-{(R)-2-[4-methoxy-3-(3-methoxy-propoxy)-benzyl]-3-methyl-butyl}-pyrrolidine-1-carboxylic acid tert-butyl ester (250 mg, 0.439 mmol) in CH₂Cl₂ (5 mL) is added, followed by stirring overnight. The reaction mixture is diluted with CH₂Cl₂ and the organic layer is washed with 1M aqueous HCl (5 mL), saturated NaHCO₃ and brine, dried (Na₂SO₄) and concentrated. The residue is purified by flash chromatography on silica gel (hexane/EtOAc 1:1) to give the title compound as colorless oil. TLC, R_{f} (hexane/EtOAc 1:1)=0.41. RP-HPLC: t_{R}=6.25 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 647.4 [M+H]⁺.

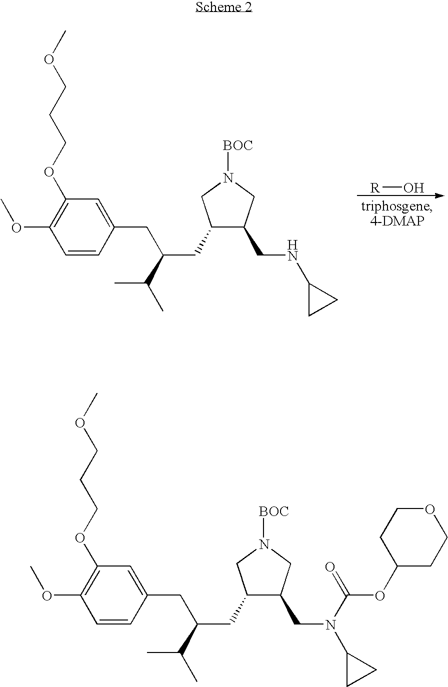

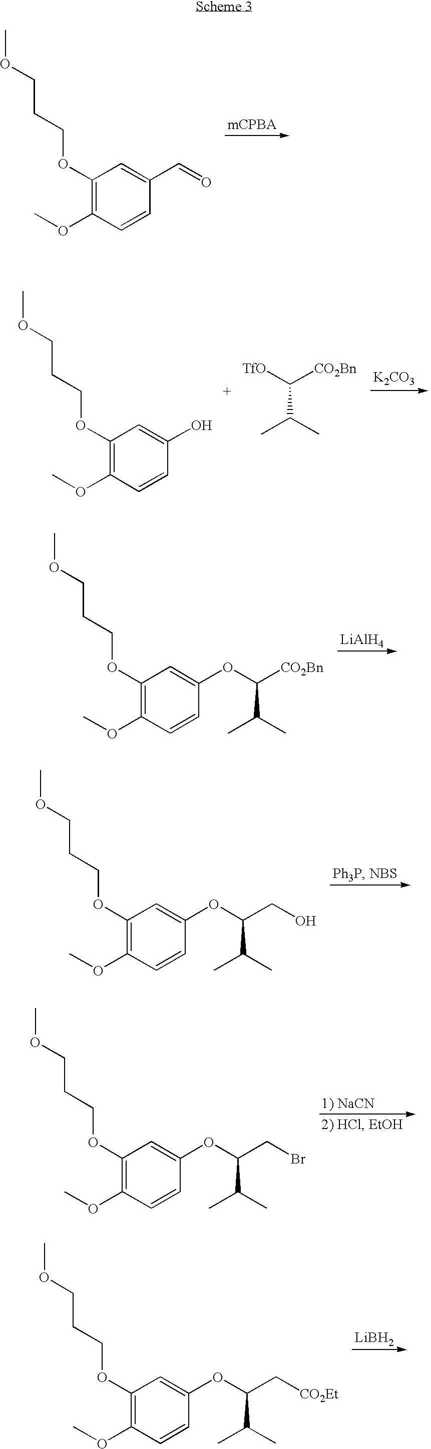

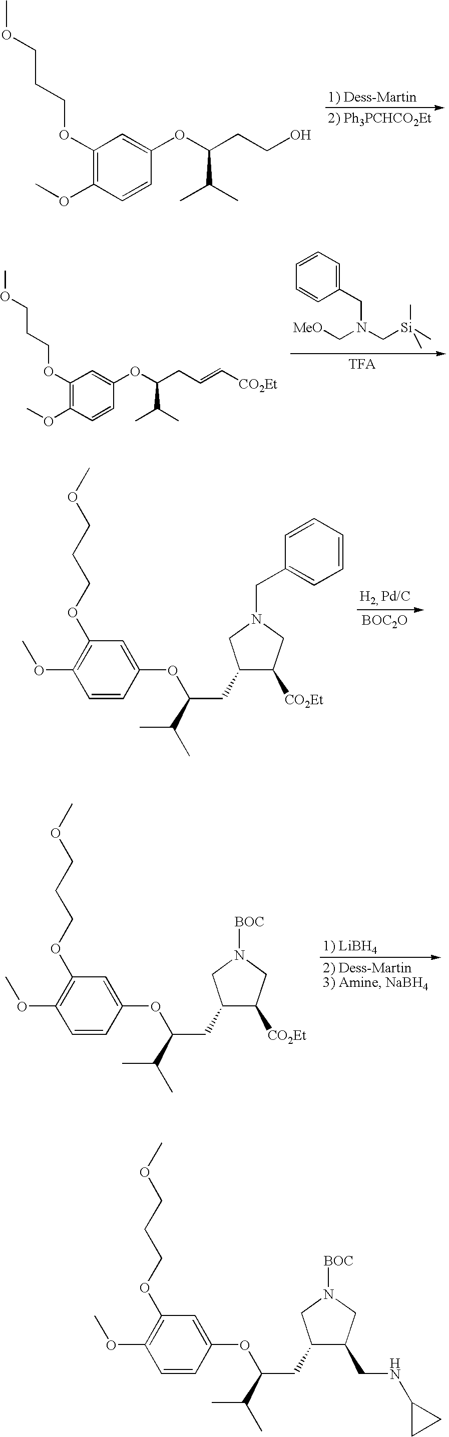

### Example 5

### N-Cyclopropyl-N-((3S*,4S*)-4-{(S)-2-[4-methoxy-3-(3-methoxy-propoxy)-phenoxy]-3-methyl-butyl}-pyrrolidin-3-ylmethyl)-2-(tetrahydro-pyran-4-yl)-acetamide

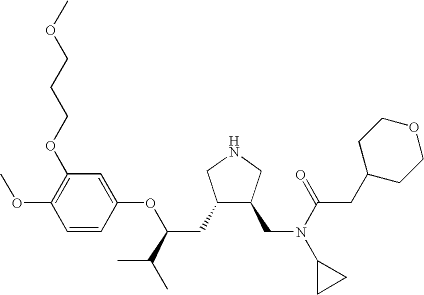

The title compound is prepared analogously as described for the title compound Example 1, starting from (3R*,4S*)-3-{[cyclopropyl-(2-tetrahydro-pyran-4-yl-acetyl)-amino]-methyl}-4-{(S)-2-[4-methoxy-3-(3-methoxy-propoxy)-phenoxy]-3-methyl-butyl}-pyrrolidine-1-carboxylic acid tert-butyl ester (200 mg, 0.31 mmol) and N—BOC deprotection in 4M HCl in dioxane (0.77 mL) in doaxane (1.0 mL) at room temperature overnight. The title compound, a mixture of two diastereoisomers, is obtained after freeze-drying as the mono hydrochloride salt. TLC, R_{f} (CH₂Cl₂/MeOH/10% NH₃ 9:1)=0.42. RP-HPLC: t_{R}=4.72 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 547.4 [M+H]⁺.

The starting materials are prepared as follows (Scheme 3):

### A. 4-Methoxy-3-(3-methoxy-propoxy)-phenol

The solution of 4-methoxy-3-(3-methoxy-propoxy)-benzaldehyde (40.0 g, 0.178 mol; the starting material is prepared as described by Goeschke et al., Helv. Chim. Acta 2003, 86, 2848-2870) and 3-chloroperbenzoic acid (61.6 g, 0.250 mol; Fluka 25800) in CH₂Cl₂ (400 mL) is refluxed for 2 hours. After cooling, the mixture is diluted with CH₂Cl₂ (100 mL) and the organic layer is washed with a saturated aqueous NaHCO₃ solution, dried (MgSO₄) and concentrated. The brownish oil (122 g) thus obtained is dissolved in a minimum amount of MeOH, followed by careful addition (exothermic) of aqueous 2M KOH (150 mL). The dark solution is stirred for 20 min at room temperature and then acidified by addition of concentrated aqueous HCl (37%). The water phase is extracted with EtOAc, the combined organics are washed with brine, dried (MgSO₄) and concentrated to afford the crude title compound as colored oil. TLC, R_{f} ( )=0. MS: 3.2 [M+H]⁺.

### B. (S)-2-Hydroxy-3-methyl-butyric acid benzyl ester

To a solution of L-α-hydroxyisovaleric acid (9.0 g, 76.2 mmol) in toluene (200 mL) is subsequently added with stirring benzylalcohol (11.8 mL, 114 mmol) and thionylchloride (1.66 mL, 22.9 mmol) and the mixture is refluxed for 36 hours. After cooling, volatiles are removed in vacuo, the residue is dissolved in EtOAc (200 mL) and the organic layer is subsequently washed with saturated NaHCO₃ (150 mL), water and brine and dried (Na₂SO₄). The crude product is purified by flash chromatography on silica gel (hexane/EtOAc 9:1) to give the title compound as colorless oil. TLC, R_{f} (hexane/EtOAc 9:1)=0.29. MS: 226.0 [M+H₂O]⁺.

### C. (S)-3-Methyl-2-trifluoromethanesulfonyloxy-butyric acid benzyl ester

To a solution of (S)-2-hydroxy-3-methyl-butyric acid benzyl ester (12.7 g, 61.0 mmol) and 2,6-lutidine (9.20 mL, 79.3 mmol) in dry CH₂Cl₂ (80 mL), cooled to −78° C. under an argon atmosphere, is added dropwise with stirring trifluoromethanesulfonic anhydride (11.3 mL, 67.1 mmol) over a 15 min period. Stirring is continued for 1 h at −70° C. before the reaction mixture is allowed to slowly warm up to room temperature during 1 h. The organics are subsequently washed with aqueous 1 N HCl (50 mL) and water, dried (Na₂SO₄) and concentrated. Flash chromatography on silica gel (hexane/EtOAc 9:1) gives the title compound as oil. TLC, R_{f} (hexane/EtOAc 9:1)=0.52. MS: 358.0 [M+H₂O]⁺.

### D. (R)-2-[4-Methoxy-3-(3-methoxy-propoxy)-phenoxy]-3-methyl-butyric acid benzyl ester

To a solution of 4-methoxy-3-(3-methoxy-propoxy)-phenol (17.6 g, 82.9 mmol) in acetone (160 mL) are added at room temperature (S)-3-methyl-2-trifluoromethanesulfonyloxy-butyric acid benzyl ester (31.0 g, 91.2 mmol) and anhydrous K₂CO₃ (14.9 g, 108 mmol) with stirring and the reaction mixture is refluxed overnight. After cooling, the solid is filtered off, washed with acetone and the combined filtrates are concentrated in vacuo. The residue is dissolved in EtOAc and the organics are subsequently washed with aqueous 1N NaOH, 1N HCl and brine. The crude product obtained after drying (Na₂SO₄) and evaporation of volatiles in vacuo is purified by flash chromatography (hexane/EtOAc 8:2) to give the title compound as yellow oil. TLC, R_{f} (hexane/EtOAc 3:1)=0.42. t_{R} (HPLC, Nucleosil C18-HD, 5-100% CH₃CN/H₂O/6 min, 100% CH₃CN/1.5 min, CH₃CN and H₂O containing 0.1% TFA, flow: 1.0 mL/min): 6.17 min. MS: 420.2 [M+H₂O]⁺.

### E. (R)-2-[4-Methoxy-3-(3-methoxy-propoxy)-phenoxy]-3-methyl-butan-1-ol

To a stirred suspension of LiAlH₄ (5.37 g, 142 mmol) in dry THF (200 mL) is added dropwise under an argon atmosphere a solution of (R)-2-[4-methoxy-3-(3-methoxy-propoxy)-phenoxy]-3-methyl-butyric acid benzyl ester (22.8 g, 56.6 mmol) in THF (200 mL) by keeping the reaction temperature below 35° C. After stirring at room temperature overnight, the reaction is quenched by subsequent dropwise addition of water (6.3 mL), a 15% aqueous NaOH solution (6.3 mL) and water (19 mL). The mixture is stirred for 1 h at room temperature, the precipitate is removed by filtration through Celite®, and the combined filtrates are concentrated. Flash chromatography on silica gel (eluent gradient hexane/EtOAc 4:1 to 1:1) gives the title compound as colorless oil. TLC, R_{f} (hexane/EtOAc 3:1)=0.18. t_{R} (HPLC, Nucleosil C18-HD, 5-100% CH₃CN/H₂O/6 min, 100% CH₃CN/1.5 min, CH₃CN and H₂O containing 0.1% TFA, flow: 1.0 mL/min): 4.59 min. MS: 299.2 [M+H]⁺.

### F. 4-((R)-1-Bromomethyl-2-methyl-propoxy)-1-methoxy-2-(3-methoxy-propoxy)-benzene

To a solution of (R)-2-[4-methoxy-3-(3-methoxy-propoxy)-phenoxy]-3-methyl-butan-1-ol (16.7 g, 56.0 mmol) in CH₂Cl₂ (320 mL) are subsequently added in portions Ph₃P (22.0 g, 84.0 mmol) and N-bromosuccinimide (14.9 g, 84.0 mmol), and stirring is continued overnight at room temperature. Volatiles are removed in vacuo and the oily residue is purified by flash chromatography (hexane/EtOAc 4:1) to give the title compound as colorless oil (which may contain minor amounts of inseparable 1-bromo-2-((R)-1-bromomethyl-2-methyl-propoxy)-5-methoxy-4-(3-methoxy-propoxy)-benzene). TLC, R. (hexane/EtOAc 3:1)=0.52. t_{R} (HPLC, Nucleosil C18-HD, 5-100% CH₃CN/H₂O/6 min, 100% CH₃CN/1.5 min, CH₃CN and H₂O containing 0.1% TFA, flow: 1.0 mL/min): 5.83 min. MS: 361.0/363.0 [M+H]⁺, 378.0/380.0 [M+H₂O]⁺.

### G. (S)-3-[4-Methoxy-3-(3-methoxy-propoxy)-phenoxy]-4-methyl-pentanenitrile

The mixture of 4-((R)-1-bromomethyl-2-methyl-propoxy)-1-methoxy-2-(3-methoxy-propoxy)-benzene (9.0 g, 24.9 mmol) and NaCN (1.47 g, 29.9 mmol) in DMSO (160 mL) is stirred overnight at room temperature. The mixture is then poured into water followed by extraction of the aqueous phase with diethyl ether. The combined organics are dried (Na₂SO₄), concentrated in vacuo, and the residue is purified by flash chromatography on silica gel (eluent hexane/EtOAc 3:1). The title compound is obtained as colorless oil. TLC, R_{f} (hexane/EtOAc 3:1)=0.26. MS: 325.2 [M+H₂O]⁺.

### H. (S)-3-[4-Methoxy-3-(3-methoxy-propoxy)-phenoxy]-4-methyl-pentanoic acid ethyl ester

A solution of (S)-3-[4-methoxy-3-(3-methoxy-propoxy)-phenoxy]-4-methyl-pentanenitrile (6.20 g, 20.2 mmol) in dry EtOH (100 mL) is saturated with anhydrous HCl gas and subsequently stirred at 70° C. for 48 hours. Volatiles are removed in vacuo, the residue is taken up in CH₂Cl₂ and the organic layer is washed with 1N NaOH, dried (Na₂SO₄) and concentrated. Purification by flash chromatography on silica gel (eluent hexane/EtOAc 85:15) gives the title compound as colorless oil. TLC, R_{f} (hexane/EtOAc 3:1)=0.62. MS: 355.2 [M+H]⁺, 372.2 [M+18]⁺.

### I. (S)-3-[4-Methoxy-3-(3-methoxy-propoxy)-phenoxy]-4-methyl-pentan-1-ol

To a solution of (S)-3-[4-methoxy-3-(3-methoxy-propoxy)-phenoxy]-4-methyl-pentanoic acid ethyl ester (4.76 g, 13.4 mmol) in dry THF (50 mL) is added under inert atmosphere LiBH₄ (0.35 g, 16.2 mmol) and the reaction mixture is stirred overnight at room temperature. After addition of another portion of LiBH₄ (0.35 g, 16.2 mmol) the reaction is continued at 60° C. for 3 hours. A 1N NaOH solution (50 mL) is added at room temperature, and the water phase is extracted with EtOAc. The combined organics are dried (Na₂SO₄), filtered and concentrated in vacuo to give the crude title compound which was used without further purification in the next reaction step. Colorless oil. TLC, R_{f} (CH₂Cl₂/MeOH 95:5)=0.44. MS: 313.2 [M+H]⁺.

### J. (E)-(S)-5-[4-Methoxy-3-(3-methoxy-propoxy)-phenoxy]-6-methyl-hept-2-enoic acid ethyl ester

To a solution of (S)-3-[4-methoxy-3-(3-methoxy-propoxy)-phenoxy]A-methyl-pentan-1-ol (3.85 g, 12.3 mmol) in CH₂Cl₂ (100 mL) is added Dess-Martin periodinane (6.27 g, 14.8 mmol; Lancaster L15779) and water (244 μL) at room temperature. After stirring for 5 min, the mixture is concentrated in vacuo to a small volume, the residue is diluted with diethyl ether (100 mL) and the organic layer is washed twice (100 mL) of a 1:1 (v/v) mixture of a saturated aqueous NaHCO₃ solution and a 10% aqueous Na₂S₂O₃ solution. The organics are dried (Na₂SO₄), volatiles are removed in vacuo and the crude aldehyde intermediate is taken up in CH₂Cl₂ (100 mL). To this solution is added ethyl(triphenylphosphoranylidene)acetate (4.44 g, 18.5 mmol; Fluka 02595) followed by stirring for 30 min at room temperature. The reaction mixture is concentrated in vacuo and the residue is purified by flash chromatography (hexane/EtOAc 4:1) to give the title compound (E-isomer; may contain minor amounts of inseparable (E)-(S)-5-[2-bromo-4-methoxy-5-(3-methoxy-propoxy)-phenoxy]-6-methyl-hept-2-enoic acid ethyl ester) as colorless oil. TLC, R_{f} (hexane/EtOAc 3:1)=0.37. t_{R} (HPLC, Nucleosil C18-HD, 5-100% CH₃CN/H₂O/6 min, 100% CH₃CN/1.5 min, CH₃CN and H₂O containing 0.1% TFA, flow: 1.0 mL/min): 5.88 min. MS: 398.2 [M+H₂O]⁺.

### K. (3S*,4S*)-1-Benzyl-4-{(S)-2-[4-methoxy-3-(3-methoxy-propoxy)-phenoxy]-3-methyl-butyl}-pyrrolidine-3-carboxylic acid ethyl ester

To a solution of (E)-(S)-5-[4-methoxy-3-(3-methoxy-propoxy)-phenoxy]-6-methyl-hept-2-enoic acid ethyl ester (3.42 g, 8.99 mmol) in toluene (20 mL), cooled to 0° C., is subsequently added under an argon atmosphere N-methoxy-N-(trimethylsilylmethyl)benzylamine (10.7 g, 44.9 mmol) and a solution of trifluoroacetic acid (3.44 mL, 44.9 mmol) in CH₂Cl₂ (2 mL) in a dropwise fashion, and stirring is continued at room temperature overnight. The reaction mixture is diluted with EtOAc, and the organic layer is washed with a saturated NaHCO₃ solution (25 mL) and 1N HCl (25 mL), dried (Na₂SO₄) and concentrated. Purification by flash chromatography on silica gel (eluent gradient hexane/EtOAc 3:1 to 1:3, then CH₂Cl₂/MeOH 95:5) gives the title compound as a mixture of trans-configured diastereomers (may be contaminated by minor amounts of (3S,4S)-1-benzyl-4-{(S)-2-[2-bromo-4-methoxy-5-(3-methoxy-propoxy)-phenoxy]-3-methyl-butyl}-pyrrolidine-3-carboxylic acid ethyl ester). Colorless oil. TLC, R_{f} (hexane/EtOAc 1:1)=0.41. t_{R} (HPLC, Nucleosil C18-HD, 5-100% CH₃CN/H₂O/6 min, 100% CH₃CN/1.5 min, CH₃CN and H₂O containing 0.1% TFA, flow: 1.0 mL/min): 5.19 min. MS: 514.2 [M+H]⁺.

### L. (3S*,4S*)-4-(S)-2-[4-Methoxy-3-(3-methoxy-propoxy)-phenoxy]-3-methyl-butyl)-pyrrolidine-1,3-dicarboxylic acid 1-tert-butyl ester 3-ethyl ester

A solution of (3S*,4S*)-1-benzyl-4-{(S)-2-[4-methoxy-3-(3-methoxy-propoxy)-phenoxy]-3-methyl-butyl}-pyrrolidine-3-carboxylic acid ethyl ester (5.13 g, 9.99 mmol) and di-tert-butyl dicarbonate (2.62 g, 12.0 mmol) in analytical grade EtOH (200 mL) is hydrogenated for 18 h in the presence of catalytic 10% Pd/C (0.51 g) at 25° C. under atmospheric pressure. The reaction mixture is filtered through Celite®, and the combined filtrates are concentrated. Purification by flash chromatography (eluent gradient hexane/EtOAc 3:1 to 1:1) gives the title compound as mixture of trans-configured diastereomers. Colorless oil. TLC, R_{f} (hexane/EtOAc 3:1)=0.36. t_{R} (HPLC, Nucleosil C18-HD, 5-100% CH₃CN/H₂O/6 min, 100% CH₃CN/1.5 min, CH₃CN and H₂O containing 0.1% TFA, flow: 1.0 mL/min): 6.28 min. MS: 541.4 [M+H₂O]⁺.

The intermediate hydrogenation product (3S*,4S*)₄-{(S)-2-[4-methoxy-3-(3-methoxy-propoxy)-phenoxy]-3-methyl-butyl}-pyrrolidine-3-carboxylic acid ethyl ester (2.60 g, 6.14 mmol) recovered by eluting the silica gel flash column by eluting with CH₂Cl₂/MeOH/10% NH₃ 9:1, is reacted with di-tert-butyl dicarbonate (1.61 g, 7.37 mmol) in presence of Et₃N (1.03 mL, 7.37 mmol) in CH₂Cl₂ (20 mL) at room temperature over 3 days. The mixture is washed with aqueous 1N HCl (40 mL) and saturated NaHCO₃, the organics are dried (Na₂SO₄) and concentrated. Purification by flash chromatography as described above affords the title compound.

### M. (3S*,4S*)-3-Hydroxymethyl-4-{(S)-2-[4-methoxy-3-(3-methoxy-propoxy)-phenoxy]-3-methyl-butyl}-pyrrolidine-1-carboxylic acid tert-butyl ester

To a solution of (3S*,4S*)₄-{(S)-2-[4-methoxy-3-(3-methoxy-propoxy)-phenoxy]-3-methyl-butyl}-pyrrolidine-1,3-dicarboxylic acid 1-tert-butyl ester 3-ethyl ester (3.25 g, 6.21 mmol) in THF (65 mL) is added LiBH₄ (0.270 g, 12.4 mmol). The reaction mixture is stirred at 60° C. overnight. After cooling to ambient temperature, 2M NaOH (50 mL) is added, and the water phase is extracted with diethyl ether. The combined organics are dried (Na₂SO₄), filtered and concentrated in vacuo to give the title compound as a ca. 1:1 mixture of trans-configured diastereoisomers. Colorless oil. TLC, R_{f} (CH₂Cl₂/MeOH 95:5)=0.38. t_{R} (HPLC, Nucleosil C18-HD, 5-100% CH₃CN/H₂O/6 min, 100% CH₃CN/1.5 min, CH₃CN and H₂O containing 0.1% TFA, flow: 1.0 mL/min): 5.93 min. MS: 482.2 [M+H]⁺; 499.4 [M+18]⁺.

### N. (3S*,4S*)-3-Formyl-4-{(S)-2-[4-methoxy-3-(3-methoxy-propoxy)-phenoxy]-3-methyl-butyl}-pyrrolidine-1-carboxylic acid tert-butyl ester

To a solution of (3S*,4S*)-3-hydroxymethyl-4-{(S)-2-[4-methoxy-3-(3-methoxy-propoxy)-phenoxy]-3-methyl-butyl}-pyrrolidine-1-carboxylic acid tert-butyl ester (0.830 g, 1.72 mmol) and Dess-Martin periodinane (0.877 g, 2.07 mmol; Lancaster L15779) in absolute CH₂Cl₂ (10 mL) is dropwise added wet CH₂Cl₂ (37 μL of water in 10 mL of CH₂Cl₂) with rigorous stirring. Stirring is continued overnight, and the reaction mixture is then concentrated in vacuo to a small volume. The residue is taken up in diethyl ether (50 mL), and the organic layer is washed with a 1:1 (v/v) mixture of a 10% aqueous Na₂S₂O₃ solution and a saturated aqueous NaHCO₃ solution (50 mL). The organic layer is dried (Na₂SO₄), filtered and concentrated to give the title compound as crude product. Colorless oil. TLC, R_{f} (CH₂Cl₂/MeOH 95:5)=0.37. RP-HPLC: t_{R}=5.77 min (Nucleosil C18-HD, 5-100% CH₃CN/H₂O/6 min, 100% CH₃CN/1.5 min, CH₃CN and H₂O containing 0.1% TFA, flow: 1.0 mL/min).

### O. (3R*,4S*)-3-Cyclopropylaminomethyl-4-{(S)-2-[4-methoxy-3-(3-methoxy-propoxy)-phenoxy]-3-methyl-butyl}-pyrrolidine-1-carboxylic acid tert-butyl ester

To a solution of (3S*,4S*)-3-formyl-4-{(S)-2-[4-methoxy-3-(3-methoxy-propoxy)-phenoxy]-3-methyl-butyl}-pyrrolidine-1-carboxylic acid tert-butyl ester (0.72 g, 1.50 mmol) in MeOH (10 mL) containing 2% of acetic acid is added cyclopropylamine (0.526 mL, 7.50 mmol). After stirring for 30 min at room temperature, NaBH₄ (0.11 g, 3.01 mmol) is added and stirring is continued for 1 h. The reaction mixture is concentrated to a small volume, followed by addition of a concentrated aqueous NaHCO₃ solution. The water phase is extracted with EtOAc, the combined organics are dried (Na₂SO₄) and evaporated in vacuo to give the title compound as crude product. Colorless oil. TLC, R_{f} (CH₂Cl₂/MeOH/10% NH₃ 9:1)=0.66. RP-HPLC: t_{R}=5.15 min (Nucleosil C18-HD, 5-100% CH₃CN/H₂O/6 min, 100% CH₃CN/1.5 min, CH₃CN and H₂O containing 0.1% TFA, flow: 1.0 mL/min). MS: 521.4 [M+H]⁺.

### P. (3R*,4S)-3-{[Cyclopropyl-(2-tetrahydro-pyran-4-yl-acetyl)-amino]-methyl}-4{(S)-2-[4-methoxy-3-(3-methoxy-propoxy)-phenoxy]-3-methyl-butyl}-pyrrolidine-1-carboxylic acid tert-butyl ester

A mixture of (3R*,4S*)-3-cyclopropylaminomethyl-4-{(S)-2-[4-methoxy-3-(3-methoxy-propoxy)-phenoxy]-3-methyl-butyl}-pyrrolidine-1-carboxylic acid tert-butyl ester (250 mg, 0.48 mmol), tetrahydropyran-4-ylacetic acid (83 mg, 0.58 mmol), 1-hydroxy-benzotriazol hydrate (78 mg, 0.58 mmol), N-(3-dimethylaminopropyl)-N′-ethyl-carbodiimide hydrochloride (110 mg, 0.58 mmol) and triethylamine (80 μL, 0.58 mmol) in CH₂Cl₂ (10 mL) is stirred at room temperature overnight. The reaction mixture is then diluted with CH₂Cl₂, and the organic layer is subsequently washed with 1M HCl (5 mL), saturated NaHCO₃ and brine, dried over Na₂SO₄ and evaporated. The residue is purified by flash chromatography on silica gel (eluent gradient:hexane/EtOAc 3:1 to 1:1) to give the title compound. RP-HPLC: t_{R}=6.10 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 647.4 [M+H]⁺.

### Example 6

### Cyclopropyl-((3S*,4S*)-4-{(S)-2-[4-methoxy-3-(3-methoxy-propoxy)-phenoxy]-3-methyl-butyl}-pyrrolidin-3-ylmethyl)-carbamic acid tetrahydro-pyran-4-yl ester

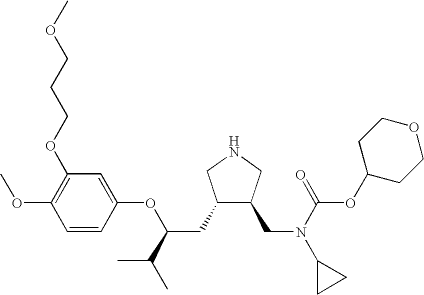

The title compound is prepared analogously as described for the title compound Example 5, starting from (3S*,4S*)-3-{[cyclopropyl-(tetrahydro-pyran-4-yloxycarbonyl)-amino]-methyl}-4-{(S)-2-[4-methoxy-3-(3-methoxy-propoxy)-phenoxy]-3-methyl-butyl}-pyrrolidine-1-carboxylic acid tert-butyl ester (240 mg, 0.37 mmol) and N—BOC deprotection in 4M HCl in dioxane (0.77 mL) in doaxane (1.0 mL) at room temperature overnight. The title compound, a mixture of two diastereoisomers, is obtained after freeze-drying as the mono hydrochloride salt. TLC, R_{f} (CH₂Cl₂/MeOH/10% NH₃ 9:1)=0.47. RP-HPLC: t_{R}=4.80 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 549.4 [M+H]⁺.

The starting material is prepared as described for Example 4A:

### (3S*,4S*)-3-{[Cyclopropyl-(tetrahydro-pyran-4-yloxycarbonyl)-amino]-methyl}-4-{(S)-2-[4-methoxy-3-(3-methoxy-propoxy)-phenoxy]-3-methyl-butyl}-pyrrolidine-1-carboxylic acid tert-butyl ester

To a solution of tetrahydro-4H-pyranol-4-ol (447 mg, 1.12 mmol) in CH₂Cl₂ (30 mL) is subsequently added triphosgene (0.154 mL, 0.38 mmol) and 4-DMAP (398 mg, 3.26 mmol), and the reaction mixture is stirred for 3 h at room temperature. Then a solution of (3R*,4S*)-3-cyclopropylaminomethyl-4-(S)-2-[4-methoxy-3-(3-methoxy-propoxy)-phenoxy]-3-methyl-butyl)pyrrolidine-1-carboxylic acid tert-butyl ester (530 mg, 1.02 mmol) in CH₂Cl₂ (5 mL) is added followed by stirring overnight. The reaction mixture is diluted with CH₂Cl₂ and the organic layer is washed with 1M aqueous HCl (25 mL), saturated NaHCO₃ and brine, dried (Na₂SO₄) and concentrated. The residue is purified by flash chromatography on silica gel (hexane/EtOAc 1:1) to give the title compound. TLC, R_{f} (hexane/EtOAc 1:1)=0.52. RP-HPLC: t_{R}=6.29 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 649.4 [M+H]⁺.

### Example 7

### N-Cyclopropyl-N-((3S*,4S*)-4-{(R)-2-[4-methoxy-3-(3-methoxy-propoxy)-benzyl]-3-methyl-butyl}-pyrrolidin-3-ylmethyl)-2-(tetrahydro-pyran-4-yl)-acetamide

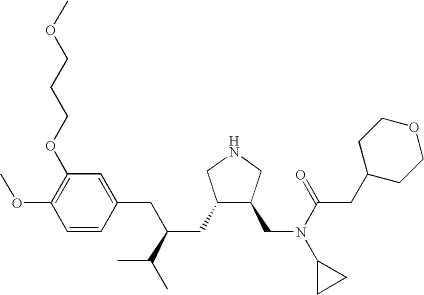

The title compound is prepared as its mono hydrochloride salt by N—BOC deprotection of (3R*,4S*)-3-{[cyclopropyl-(2-tetrahydro-pyran-4-yl-acetyl)-amino]-methyl}-4-{(R)-2-[4-methoxy-3-(3-methoxy-propoxy)-benzyl]-3-methyl-butyl}-pyrrolidine-1-carboxylic acid tert-butyl ester with 4M HCl in dioxane according to the method described for Example 1. RP-HPLC: t_{R}=4.90 min (Nucleosil C18-HD column, 10-100% CH₃CN/H₂O/5 min, 100% CH₃CN/3 min, CH₃CN and H₂O containing 0.1% TFA, flow: 1.5 mL/min; column: 4×70 mm; particle size 3 μm). MS: 545.4 [M+H]⁺.

The starting material (3R*,4S*)-3-{[cyclopropyl-(2-tetrahydro-pyran-4-yl-acetyl)-amino]-methyl}-4-{(R)-2-[4-methoxy-3-(3-methoxy-propoxy)-benzyl]-3-methyl-butyl}-pyrrolidine-1-carboxylic acid tert-butyl ester is obtained by the coupling reaction described in Example 1J from (3R*,4S*)-3-cyclopropylaminomethyl-4-{(R)-2-[4-methoxy-3-(3-methoxy-propoxy)-benzyl]-3-methyl-butyl}-pyrrolidine-1-carboxylic acid tert-butyl ester and tetrahydropyran-4-yl-acetic acid RP-HPLC: t_{R}=6.38 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 645.5 [M+H]⁺.

### Example 8

### 5-Methyl-pyrazine-2-carboxylic acid cyclopropyl-((3S*,4S*)-4-{(R)-2-[4-methoxy-3-(3-methoxy-propoxy)-benzyl]-3-methyl-butyl}-pyrrolidin-3-ylmethyl)-amide

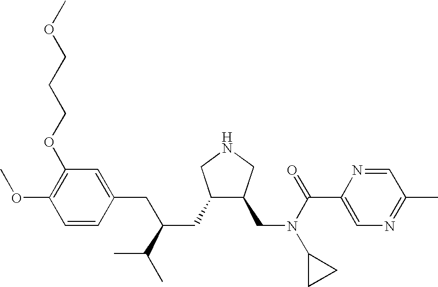

The title compound is prepared as its mono hydrochloride salt by N—BOC deprotection of (3R*,4S*)-3-{[cyclopropyl-(5-methyl-pyrazine-2-carbonyl)-amino]-methyl}-4{(R)-2-[4-methoxy-3-(3-methoxy-propoxy)-benzyl]-3-methyl-butyl}-pyrrolidine-1-carboxylic acid tert-butyl ester with 4M HCl in dioxane according to the method described for Example 1. RP-HPLC: t_{R}=4.79 min (Nucleosil C18-HD column, 10-100% CH₃CN/H₂O/5 min, 100% CH₃CN/3 min, CH₃CN and H₂O containing 0.1% TFA, flow: 1.5 mL/min; column: 4×70 mm; particle size 3 μm). MS: 539.4 [M+H]⁺.

The starting material (3R*,4S*)-3-{[cyclopropyl-(5-methyl-pyrazine-2-carbonyl)-amino]-methyl}-4-{(R)-2-[4-methoxy-3-(3-methoxy-propoxy)-benzyl]-3-methyl-butyl}-pyrrolidine-1-carboxylic acid tert-butyl ester is obtained by the coupling reaction described in Example 1J from (3R*,4S*-3-cyclopropylaminomethyl-4-{(R)-2-[4-methoxy-3-(3-methoxy-propoxy)-benzyl]-3-methyl-butyl}-pyrrolidine-1-carboxylic acid tert-butyl ester and 5-methylpyrazine-2-carboxylic acid RP-HPLC: t_{R}=6.27 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 639.4 [M+H]⁺.

### Example 9

### Tetrahydro-pyran-4-carboxylic acid cyclopropyl-((3S*,4S*)-4-{(R)-2-[4-methoxy-3-(3-methoxy-propoxy)-benzyl]-3-methyl-butyl}-pyrrolidin-3-ylmethyl)-amide

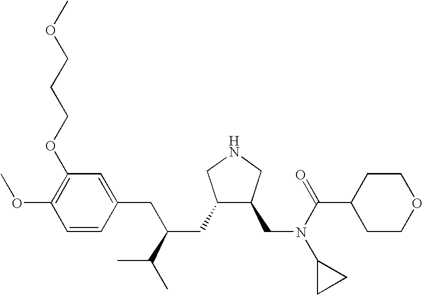

The title compound is prepared as its mono hydrochloride salt by N—BOC deprotection of (3R*,4S*)-3-{[cyclopropyl-(tetrahydro-pyran-4-carbonyl)-amino]-methyl}-4-{(R)-2-[4-methoxy-3-(3-methoxy-propoxy)-benzyl]-3-methyl-butyl}-pyrrolidine-1-carboxylic acid tert-butyl ester with 4M HCl in dioxane according to the method described for Example 1. RP-HPLC: t_{R}=4.79 min (Nucleosil C18-HD column, 10-100% CH₃CN/H₂O/5 min, 100% CH₃CN/3 min, CH₃CN and H₂O containing 0.1% TFA, flow: 1.5 mL/min; column: 4×70 mm; particle size 3 μm). MS: 531.4 [M+H]⁺.

The starting material (3R*,4S*)-3-{[cyclopropyl-(tetrahydro-pyran-4-carbonyl)-amino]-methyl}) 4-{(R)-2-[4-methoxy-3-(3-methoxy-propoxy)-benzyl]-3-methyl-butyl}-pyrrolidine-1-carboxylic acid tert-butyl ester is obtained by the coupling reaction described in Example 1J from (3R*,4S*)-3-cyclopropylaminomethyl-4-{(R)-2-[4-methoxy-3-(3-methoxy-propoxy)-benzyl]-3-methyl-butyl}pyrrolidine-1-carboxylic acid tert-butyl ester and tetrahydropyran-4-yl-carboxylic acid RP-HPLC: t_{R}=6.25 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 631.5 [M+H]⁺.

### Example 10

### Cyclopropyl-((3S,4S)-4-{(R)-2-[4-methoxy-3-(3-methoxy-propoxy)-phenoxy]-3-methyl-butyl}-pyrrolidin-3-ylmethyl)-carbamic acid tetrahydro-pyran-4-yl ester

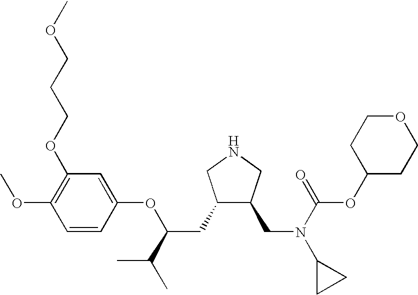

The title compound is prepared, according to Example 6, starting from (3S,4S)-3-{[cyclopropyl-(tetrahydro-pyran-4-yloxycarbonyl)-amino]-methyl}-4-{(S)-2-[4-methoxy-3-(3-methoxy-propoxy)-phenoxy]-3-methyl-butyl}-pyrrolidine-1-carboxylic acid tert-butyl ester (290 mg, 0.45 mmol) and N—BOC deprotection in 4M HCl in dioxane (1.1 mL) in dioxane (1.0 mL) at room temperature overnight. The title compound is obtained after freeze-drying as the mono hydrochloride salt. RP-HPLC: t_{R}=4.75 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 549.4 [M+H]⁺.

The starting material (3S,4S)-3-{[cyclopropyl-(tetrahydro-pyran-4-yloxycarbonyl)-amino]-methyl}-4-{(S)-2-[4-methoxy-3-(3-methoxy-propoxy)-phenoxy]-3-methyl-butyl}-pyrrolidine-1-carboxylic acid tert-butyl ester is prepared according to the methods described in Examples 5N to 5P from (3S,4S)-3-hydroxymethyl-4-{(S)-2-[4-methoxy-3-(3-methoxy-propoxy)-phenoxy]-3-methyl-butyl}-pyrrolidine-1-carboxylic acid tert-butyl ester.

### A. (3S,4S)-3-Hydroxymethyl-4-{(S)-2-[4-methoxy-3-(3-methoxy-propoxy)-phenoxy]-3-methyl-butyl}-pyrrolidine-1-carboxylic acid tert-butyl ester

The title compounds is obtained by preparative HPLC separation of the mixture of the corresponding (3S,4S,4′S)- and (3R,4R,4′S)-diastereomers (described in Example 5M), using cellulose-3,5-dichlorophenylcarbamate as the stationary phase (20 μm particle size; column size: 48×185 mm); eluent:n-hexane/EtOH 85:15; flow rate 60 mL/min; detection: 290 nm (UV). The trans-configured title compound is obtained as colorless oil. t_{R} (HPLC, cellulose-3,5-dichlorophenylcarbamate 20 lam; column size 4×25 mm; eluent:n-hexane/EtOH 85:15; flow rate 1.0 mL/min; detection: 290 nm (UV)): 11.4 min. MS: 482.2 [M+H]⁺; 499.4 [M+18]⁺.

### B. (3R,4R)-3-Hydroxymethyl-4-{(S)-2-[4-methoxy-3-(3-methoxy-propoxy)-phenoxy]-3-methyl-butyl}-pyrrolidine-1-carboxylic acid tert-butyl ester

The title compound is obtained as described in Example 10A by preparative HPLC separation. Colorless oil. t_{R} (HPLC, cellulose-3,5-dichlorophenylcarbamate 20 μm; column size 4×25 mm; eluent:n-hexane/EtOH 85:15; flow rate 1.0 mL/min; detection: 290 nm (UV)): 10.0 min. MS: 482.2 [M+H]⁺; 499.4 [M+18]⁺.

### Example 11

### Cyclopropyl-((3R,4R)-4-(S)-2-[4-methoxy-3-(3-methoxy-propoxy)-phenoxy]-3-methyl-butyl)-pyrrolidin-3-ylmethyl)-carbamic acid tetrahydro-pyran-4-yl ester

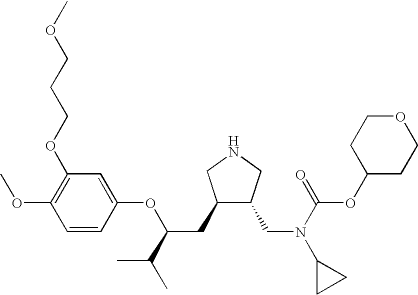

The title compound is obtained by the methods described for Example 10. RP-HPLC: t_{R}=4.77 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 549.4 [M+H]⁺.

### Example 12

### N-Cyclopropyl-N-((3S,4S)-4{(S)-2-[4-methoxy-3-(3-methoxy-propoxy)-phenoxy]-3-methyl-butyl}-pyrrolidin-3-ylmethyl)-2-(tetrahydro-pyran-4-yl)-acetamide

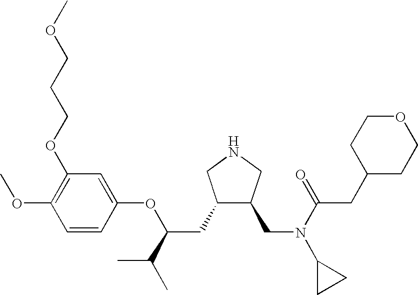

The title compound is prepared as described in Example 10, starting from (3R,4S)-3-{[cyclopropyl-(tetrahydro-pyran-4-yloxycarbonyl)-amino]-methyl)-4-{(S)-2-[4-methoxy-3-(3-methoxy-propoxy)-phenoxy]-3-methyl-butyl}-pyrrolidine-1-carboxylic acid tert-butyl ester (180 mg, 0.28 mmol; Example 10A) and N—BOC deprotection in 4M HCl in dioxane (0.70 mL) in dioxane (1.0 mL) at room temperature overnight. The title compound is obtained after freeze-drying as the mono hydrochloride salt. RP-HPLC: t_{R}=4.64 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 547.4 [M+H]⁺.

### Example 13

### N-Cyclopropyl-N-((3R,4R)-4{(S)-2-[4-methoxy-3-(3-methoxy-propoxy)-phenoxy]-3-methyl-butyl}-pyrrolidin-3-ylmethyl)-2-(tetrahydro-pyran-4-yl)-acetamide

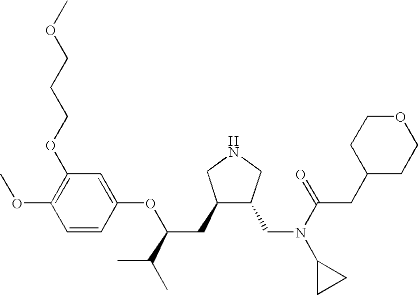

The title compound is prepared as described in in Example 10, starting from (3S,4R)-3-{[cyclopropyl-(tetrahydro-pyran-4-yloxycarbonyl)-amino]-methyl}-4-{(S)-2-[4-methoxy-3-(3-methoxy-propoxy)-phenoxy]-3-methyl-butyl}-pyrrolidine-1 garb oxylic acid tert-butyl ester (195 mg, 0.30 mmol; Example 10B) and N—BOC deprotection in 4M HCl in dioxane (0.75 mL) in dioxane (1.0 mL) at room temperature overnight. The title compound is obtained after freeze-drying as the mono hydrochloride salt. RP-HPLC: t_{R}=4.67 min (Nucleosil C18-HD column, 5-100% CH₃CN/H₂O/6 min, 100% CH₃CN/1.5 min, CH₃CN and H₂O containing 0.1% TFA, flow: 1.0 mL/min). MS: 547.4 [M+H]⁺.

### Example of Formulation 1: Soft Capsules

5000 soft gelatin capsules, each comprising as active ingredient 0.05 g of any one of the compounds of formula I mentioned in any one of the preceding Examples, are prepared as follows:

1. Composition

| | | |
|---|---|---|
| Active ingredient | 250 | g |
| Lauroglycol | 2 | liters |

Preparation process: The pulverized active ingredient is suspended in Lauroglykol® (propylene glycol laurate, Gattefosse S.A., Saint Priest, France) and ground in a w et pulverizer to produce a particle size of about 1 to 3 μm. 0.419 g portions of the mixture are then introduced into soft gelatin capsules using a capsule-filling machine.

### Example of Formulation 2: Tablets Comprising Compounds of the Formula I

Tablets, comprising, as active ingredient, 100 mg of any one of the compounds of formula I in any one of the preceding Examples are prepared with the following composition, following standard procedures:

Composition

| | | |
|---|---|---|
| Active Ingredient | 100 | mg |
| crystalline lactose | 240 | mg |
| Avicel | 80 | mg |
| PVPPXL | 20 | mg |
| Aerosil | 2 | mg |
| magnesium stearate | 5 | mg |
| | 447 | mg |

Manufacture: The active ingredient is mixed with the carrier materials and compressed by means of a tabletting machine (Korsch EKO, stamp diameter 10 mm).

Avicel® is microcrystalline cellulose (FMC, Philadelphia, USA). PVPPXL is polyvinyl-polypyrrolidone, cross-linked (BASF, Germany). Aerosil® is silicon dioxide (Degussa, Germany).

1. A compound of the formula I 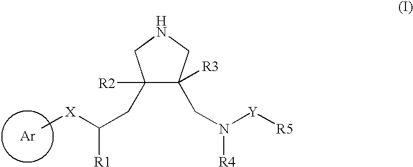
wherein
R¹ is unsubstituted or substituted alkyl or substituted or unsubstituted cycloalkyl;
R² and R³ are independently of each other hydrogen, alkoxy, alkyl, hydroxy or halogen;
R⁴ is unsubstituted or substituted alkyl or substituted or unsubstituted cycloalkyl;
R⁵ is unsubstituted or substituted alkyl, substituted or unsubstituted heterocyclyl, unsubstituted or substituted or unsubstituted aryl, or substituted or unsubstituted cycloalkyl;
X is CH₂ or O;
Y is —(CO)—, —S(O)₂— or —C(O)O—; and
Ar is unsubstituted or substituted aryl or unsubstituted or substituted mono- or bicyclic aromatic heterocyclyl;
or a salt thereof.

2. A compound of the formula I according to claim 1,
wherein
R¹ is unsubstituted or substituted alkyl or substituted or unsubstituted cycloalkyl;
R² and R³ are independently of each other hydrogen, alkoxy, alkyl, hydroxy or halogen;
R⁴ is unsubstituted or substituted alkyl or substituted or unsubstituted cycloalkyl;
R⁵ is unsubstituted or substituted alkyl, substituted or unsubstituted heterocyclyl, unsubstituted or substituted or unsubstituted aryl, or substituted or unsubstituted cycloalkyl, substituted or unsubstituted heterocyclyl-alkyl, unsubstituted or substituted or unsubstituted aryl-alkyl, or substituted or unsubstituted cycloalkyl-alkyl;
X is CH₂ or 0;
Y is —(CO)—, —S(O)₂— or —C(O)O—; and
Ar is unsubstituted or substituted aryl or unsubstituted or substituted mono- or bicyclic aromatic heterocyclyl;
where in each case of occurrence above in this claim
unsubstituted or substituted aryl is mono- or polycyclic, especially monocyclic, bicyclic, tricyclic aryl with 6 to 22 carbon atoms, especially phenyl, naphthyl, indenyl or fluorenyl, and is unsubstituted or substituted by one or more, especially one to three, moieties, preferably independently selected from the group consisting of:
a substitutent of the formula —(C₀-C₇-alkylene)-(X)ᵣ—(C₁-C₇-alkylene)-(Y)ₛ—(C₀-C₇-alkylene)-H where Co-alkylene means that a bond is present instead of bound alkylene, r and s, each independently of the other, are 0 or 1 and each of X and Y, if present and independently of each other, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, or is phenyl, naphthyl, phenyl- or naphthyl-C₁-C₇-alkyl and halo-C₁-C₇-alkyl; where said substituent of formula —(C₀-C₇-alkylene)-(X)ᵣ—(C₁-C₇-alkylene)-(Y)ₛ—(C₀-C₇-alkylene)-His preferably C₁-C₇-alkyl, hydroxy-C₁-C₇-alkyl, C₁-C₇alkoxy-C₁-C₇-alkyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, (N—) mono- or (N,N-) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇-alkoxy, C₁-C₇-alkanoyloxy, mono- or di-(C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N— mono- or N,N-di-(C₁-C₇-alkyl)-aminocarbonyl, N—C₁-C₇-alkoxy-C₁-C₇-alkylcarbamoyl and N-mono- or N,N-di-(C₁-C₇-alkyl)-aminosulfonyl;
C₂-C₇-alkenyl, C₂-C₇-alkinyl, phenyl, naphtyl, cycloalkyl heterocyclyl, especially as defined below for heterocyclyl, preferably selected from pyrrolyl, furanyl, thienyl, pyrimidine-2,4-dione-1-, -3- or -5-yl and benzo[1,3]-dioxolyl, phenyl- or naphthyl- or heterocyclyl-C₁-C₇-alkyl wherein heterocyclyl is as defined below preferably selected from pyrrolyl, furanyl, thienyl, pyrimidine-2,4-dione-1-, -3- or -5-yl and benzo[1,3]-dioxolyl, such as benzyl or naphthylmethyl, halo-C₁-C₇-alkyl, such as trifluoromethyl, phenyloxy- or naphthyloxy-C₁-C₇-alkyl, cycloalkyl-C₁-C₇-alkyl, heterocyclyl-C₁-C₇-alkyl, phenyl-C₁-C₇-alkoxy- or naphthyl-C₁-C₇-alkoxy-C₁-C₇-alkyl cycloalkyl-C₁-C₇-alkoxy-C₁-C₇-alkyl, heterocyclyl-C₁-C₇-alkoxy-C₁-C₇-alkyl, di-(naphthyl- or phenyl)-amino-C₁-C₇-alkyl mono- or di-(heterocyclyl-, cycloalkyl-, naphthyl- or phenyl)-amino-C₁-C₇-alkyl, di-(naphthyl- or phenyl-C₁-C₇-alkyl)-amino-C₁-C₇-alkyl, mono- or di-(heterocyclyl-, cycloalkyl-, naphthyl- or phenyl-C₁-C₇-alkyl)-amino-C₁-C₇-alkyl, benzoyl- or naphthoylamino-C₁-C₇-alkyl, cycloalkyl-COamino-C₁-C₇-alkyl, heterocyclyl-COamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, cycloalkylsulfonylamino-C₁-C₇-alkyl, heterocyclylsulfonylamino-C₁-C₇-alkyl, phenyl- or naphthyl-C₁-C₇-alkylsulfonylamino-C₁-C₇-alkyl, cycloalkyl-C₁-C₇-alkylsulfonylamino-C₁-C₇-alkyl, heterocyclyl-C₁-C₇-alkylsulfonylamino-C₁-C₇-alkyl, carboxy-C₁-C₇-alkyl, halo, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, cycloalkyl-C₁-C₇-alkoxy, heterocyclyl-C₁-C₇-alkoxy, phenyl- or naphthyloxy, cycloalkyloxy, heterocyclyloxy, phenyl- or naphthyl-C₁-C₇-alkyloxy, cycloalkyl-C₁-C₇-alkyloxy, heterocyclyl-C₁-C₇-alkyloxy, benzoyl- or naphthoyloxy, halo-C₁-C₇-alkylthio, such as trifluoromethylthio, phenyl- or naphthylthio, cycloalkylthio, heterocyclylthio, phenyl- or naphthyl-C₁-C₇-alkylthio, cycloalkyl-C₁-C₇-alkylthio, heterocyclyl-C₁-C₇-alkylthio, benzoyl- or naphthoylthio, nitro, amino, mono- or di-(naphthyl- or phenyl-C₁-C₇-alkyl)-amino, mono- or di-(heterocyclyl-, cycloalkyl-, naphthyl- or phenyl-C₁-C₇-alkyl)-amino, benzoyl- or naphthoylamino, phenyl- or naphthylsulfonylamino wherein phenyl or naphthyl is unsubstituted or substituted by one or more, especially one to three, C₁-C₇-alkyl moieties, cycloalkylsulfonylamino, heterocyclylsulfonylamino, phenyl- or naphthyl-C₁-C₇-alkylsulfonylamino, cycloalkyl-C₁-C₇-alkylsulfonylamino, heterocyclyl-C₁-C₇-alkylsulfonylamino, carboxyl, C₁-C₇-alkyl-carbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N—) mono- or (N,N-) di-(C₁-C₇-alkyl)-amino-C₁-C₇-alkylcarbonyl, C₁-C₇-alkanoylamino-C₁-C₇-alkylcarbonyl, N-mono or (N,N-) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, N-mono or (N,N-) di-(C₁-C₇-alkyl)-amino-C₁-C₇-alkoxycarbonyl, carbamoyl, N-mono or N,N-di-(heterocyclyl-, cycloalkyl-, naphthyl- or -phenyl-)-aminocarbonyl, N-mono- or N,N-di-(heterocyclyl-, cycloalkyl-, naphthyl- or phenyl-C₁-C₇-alkyl)-aminocarbonyl, cyano, C₁-C₇-alkylene which is unsubstituted or substituted by up to four C₁-C₇-alkyl substituents and bound to two adjacent ring atoms of the aryl moiety, C₂-C₇-alkenylene or -alkinylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkyl moieties, cycloalkylsulfinyl, heterocyclylsulfinyl, phenyl- or naphthyl-C₁-C₇-alkylsulfinyl, cycloalkyl-C₁-C₇-alkylsulfinyl, heterocyclyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, N-mono or (N,N-) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkyl moieties, cycloalkylsulfonyl, heterocyclylsulfonyl, phenyl- or naphthyl-C₁-C₇-alkylsulfonyl, cycloalkyl-C₁-C₇-alkylsulfonyl, heterocyclyl-C₁-C₇-alkylsulfinyl, sulfamoyl and N-mono or N,N-di-(C₁-C₇-alkyl, phenyl-, naphthyl, heterocyclyl, cycloalkyl, phenyl-C₁-C₇-alkyl and/or naphthyl-C₁-C₇-alkyl, heterocyclyl-C₁-C₇-alkyl, cycloalkyl-C₁-C₇-alkyl)-aminosulfonyl;
unsubstituted or substituted heterocyclyl is a mono- or bicyclic, unsaturated, partially saturated or saturated ring system with preferably 3 to 22 (more preferably 3 to 14) ring atoms and with one or more, preferably one to four, heteroatoms independently selected from nitrogen (═N—, —NH— or substituted —NH—), oxygen, sulfur (—S—, S(═O)— or S—(═O)₂—) which is unsubstituted or substituted by one or more, e.g. up to three, substitutents preferably independently selected from the substitutents mentioned above for aryl and from oxo, preferably selected from the following moieties: 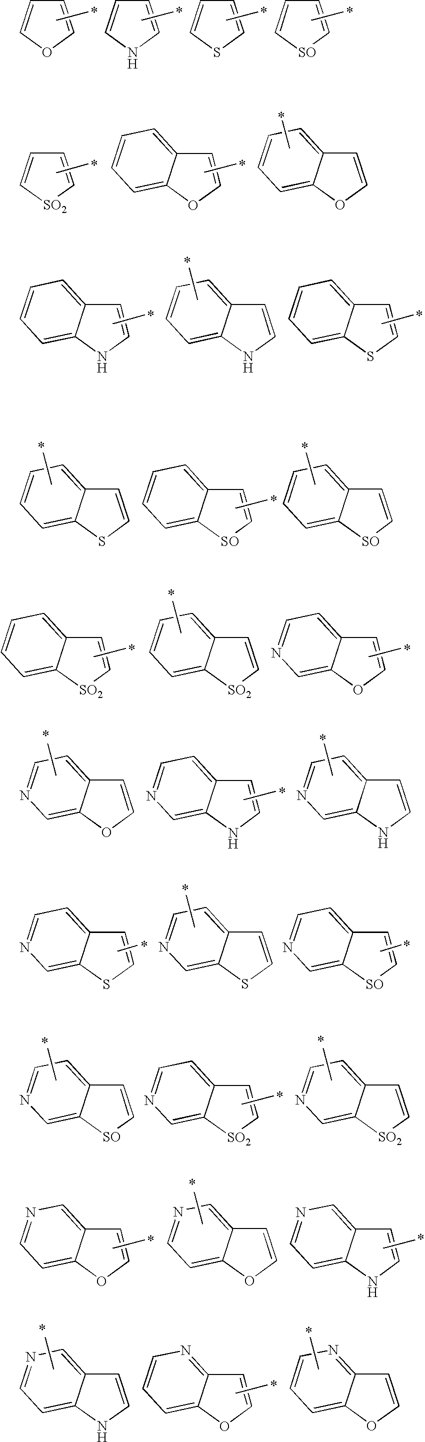 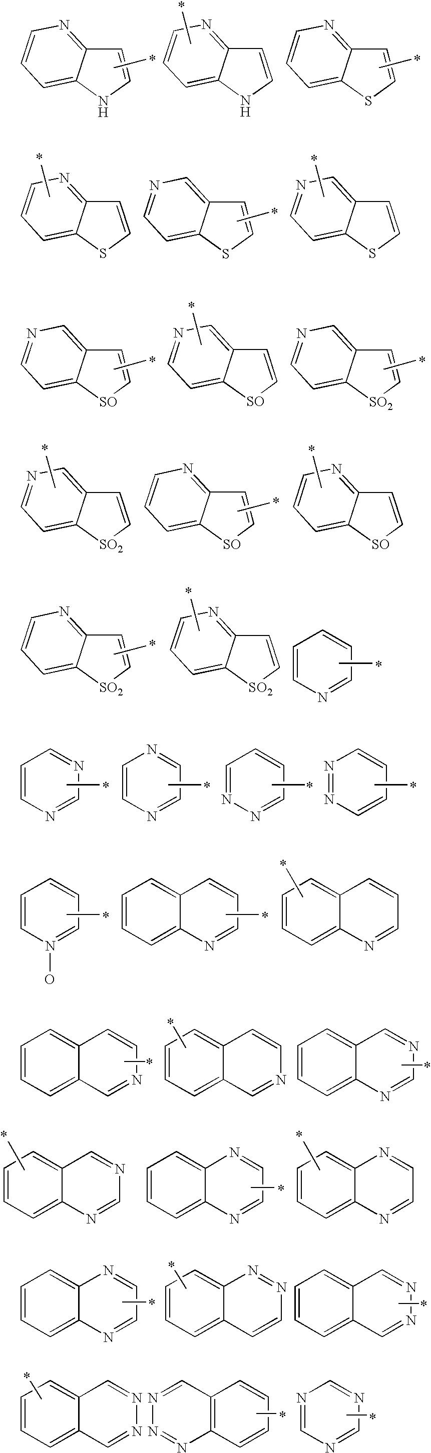 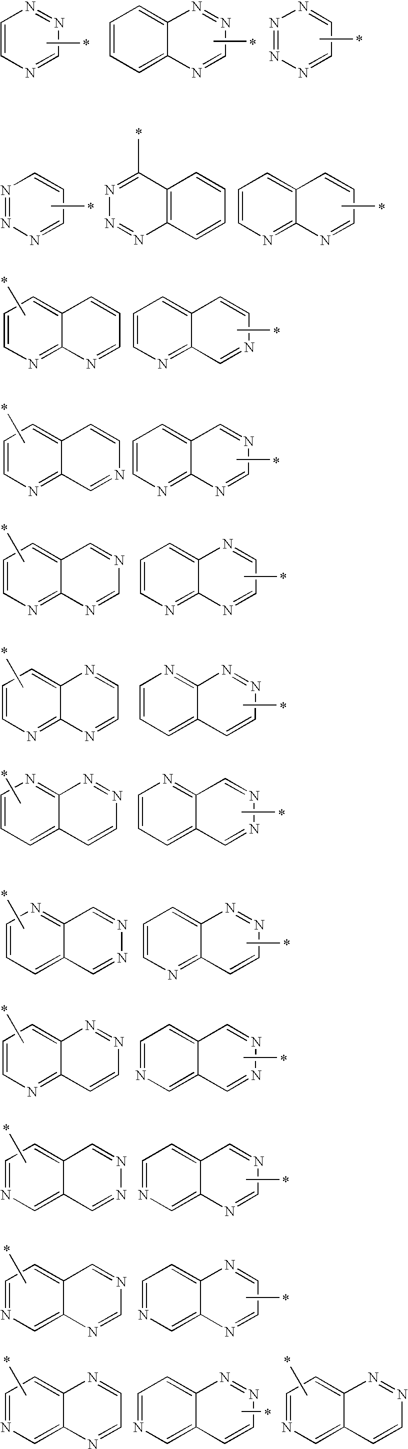 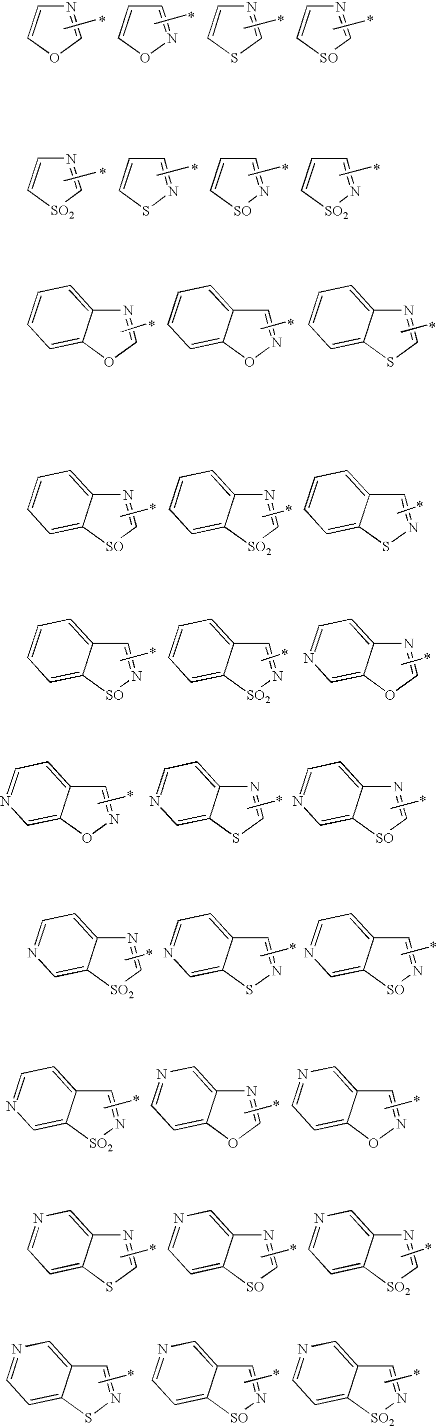 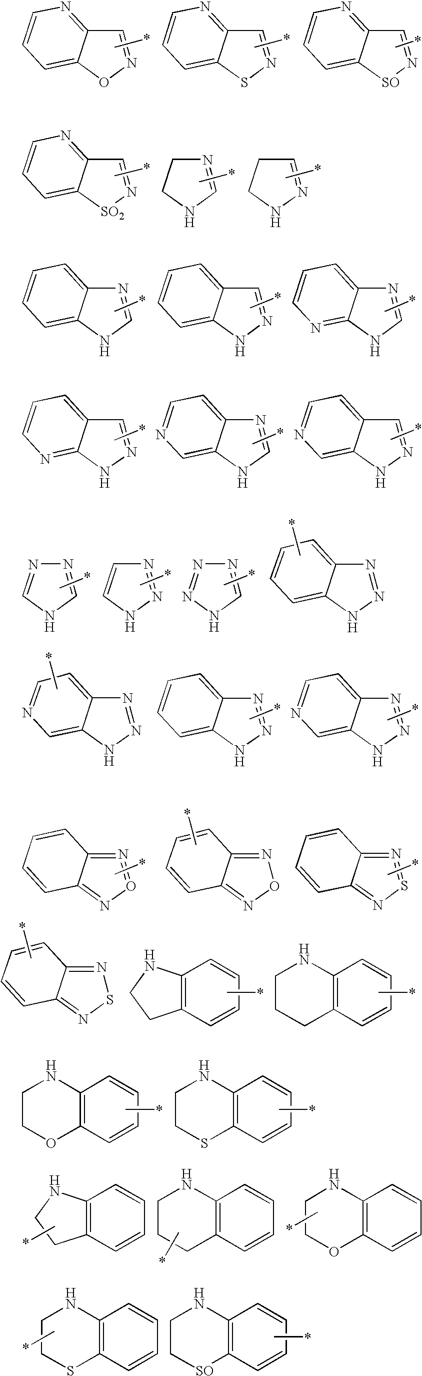 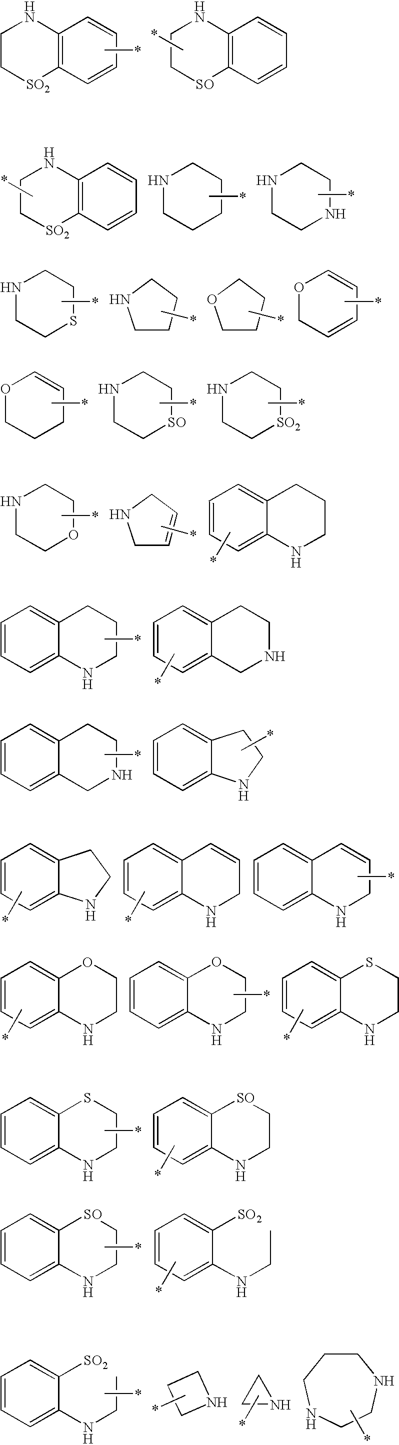 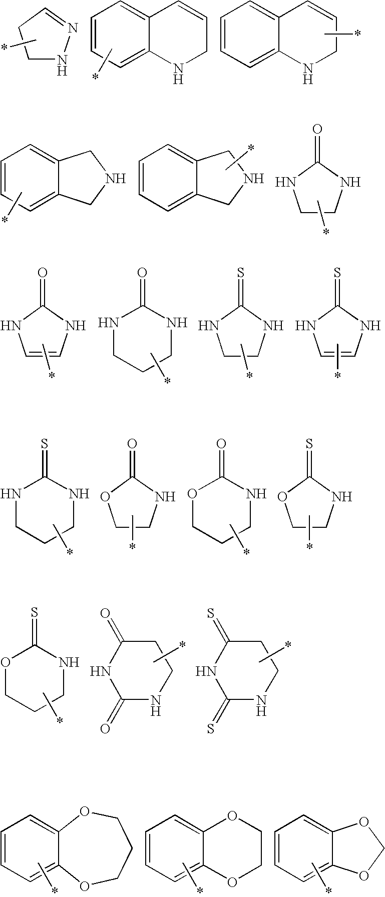
where in each case where an NH is present the bond with the asterisk connecting the respective heterocyclyl moiety to the rest of the molecule the H may be replaced with said bond and/or the H may be replaced by a substituent,
unsubstituted or substituted cycloalkyl is mono- or polycyclic, more preferably monocyclic, C₃-C₁₀-cycloalkyl which may include one or more double (e.g. in cycloalkenyl) and/or triple bonds (e.g. in cycloalkinyl), and is unsubstituted or substituted by one or more, e.g. one to three substitutents preferably independently selected from those mentioned above as substituents for aryl;
unsubstituted or substituted alkyl is C₁-C₂₀-alkyl, more preferably C₁-C₇-alkyl, that is straight-chained or branched, which is unsubstituted or substituted by one or more, e.g. up to three moieties selected from unsubstituted or substituted aryl as described above, especially phenyl or naphthyl each of which is unsubstituted or substituted as described above for unsubstituted or substituted aryl, unsubstituted or substituted heterocyclyl as described above, especially pyrrolyl, furanyl, thienyl, pyrimidine-2,4-dione-1-, -2-, -3- or -5-yl and benzo[1,3]dioxolyl, each of which is unsubstituted or substituted as described above for unsubstituted or substituted heterocyclyl; unsubstituted or substituted cycloalkyl as described above, especially cyclopropyl, cyclobutyl, cyclopentyl or cyclohexyl each of which is unsubstituted or substituted as described above for unsubstituted or substituted cycloalkyl; C₂-C₇-alkenyl, C₂-C₇-alkinyl, halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkylthio, such as trifluoromethylthio, hydroxy-C₁-C₇-alkylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl, hydroxy-C₁-C₇-alkyl and/or C₁-C₇-alkoxy-C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N— mono- or N,N-di-(C₁-C₇-alkyl)-aminocarbonyl, N-mono- or N,N-di-(naphthyl- or phenyl-C₁-C₇-alkyl)-aminocarbonyl, N-mono- or N,N-di-(alkyl, naphtyl, phenyl, heterocyclyl, cycloalkyl, naphthyl-, heterocyclyl-, cycloalkyl- or phenyl-C₁-C₇-alkyl)-aminocarbonyl, cyano, C₁-C₇-alkenylene or -alkinylene, C₁-C₇-alkylenedioxy, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkyl moieties, cycloalkylsulfinyl, heterocyclylsulfinyl, phenyl- or naphthyl-C₁-C₇-alkylsulfinyl, cycloalkyl —C₁-C₇-alkylsulfinyl, heterocyclyl —C₁-C₇-alkylsulfinyl, sulfonyl, C₁-C₇-alkylsulfonyl, phenyl- or naphthylsulfonyl wherein phenyl or naphthyl is unsubstituted or substituted by one or more, especially one to three, C₁-C₇-alkyl moieties, cycloalkylsulfonyl, heterocyclylsulfonyl, phenyl- or naphthyl-C₁-C₇-alkylsulfonyl, cycloalkyl —C₁-C₇-alkylsulfonyl, heterocyclyl —C₁-C₇-alkylsulfonyl, sulfamoyl, N-mono- or N,N-di-(alkyl, naphtyl, phenyl, heterocyclyl, cycloalkyl, naphthyl-, heterocyclyl-, cycloalkyl- or phenyl-C₁-C₇-alkyl)-aminosulfonyl, N-mono-, N′-mono-, N,N-di- or N,N,N′-tri-(C₁-C₇-alkyl, hydroxy-C₁-C₇-alkyl and/or C₁-C₇-alkoxy-C₁-C₇-alkyl)-aminocarbonylamino and N-mono-, N′-mono-, N,N-di- or N,N,N′-tri-(C₁-C₇-alkyl, hydroxy-C₁-C₇-alkyl and/or C₁-C₇-alkoxy-C₁-C₇-alkyl)aminosulfonylamino or a pharmaceutically acceptable salt thereof.

3. A compound of the formula I according to claim 1 or 2, wherein
R¹ is C₁-C₇-alkyl or C₃-C₁₀-cycloalkyl.

4. A compound of the formula I according to any of the preceding claims, wherein
R² and R³ are independently of each other hydrogen.

5. A compound of the formula I according to any of the preceding claims, wherein
R⁴ is C₁-C₇-alkyl or C₃-C₁₀-cycloalkyl.

6. A compound of the formula I according to any of the preceding claims, wherein
R⁵ is unsubstituted or substituted alkyl, substituted or unsubstituted heterocyclyl, unsubstituted or substituted or unsubstituted aryl, or substituted or unsubstituted cycloalkyl, wherein each is unsubstituted or substituted by one to three, substitutents selected from the group consisting of
halo, phenyl or naphthyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl, cyano, C₁-C₇-alkyl and substituted or unsubstituted heterocyclyl.

7. A compound of the formula I according to any of the preceding claims, wherein
R⁵ is C₁-C₇-alkyl or 5- to 10-membered mono- or bicyclic heterocyclyl containing at last one heteroatom selected from O, N or S, wherein each is unsubstituted or substituted by one or more, e.g. up to three, substitutents selected from the group consisting of
halo, phenyl or naphthyl, hydroxy, C₁-C₇-alkoxy, amino, mono- or di-(C₁-C₇-alkyl)-amino, C₁-C₇-alkanoylamino, C₁-C₇-alkyl-sulfonylamino, phenyl- or napthylsulfonylamino, phenyl- or naphthyl-C₁-C₇-alkylsulfonylamino, C₁-C₇-alkoxy-C₁-C₇-alkoxy, hydroxy-C₁-C₇-alkoxy, phenyl- or naphthyloxy, phenyl- or naphthyl-C₁-C₇-alkyloxy, C₁-C₇-alkanoyloxy, nitro, carboxyl, C₁-C₇-alkoxy-carbonyl, phenyl- or naphthyl-C₁-C₇-alkoxycarbonyl, carbamoyl, N-mono- or N,N-di-(C₁-C₇-alkyl-, phenyl-, naphthyl-, phenyl-C₁-C₇-alkyl- or naphthyl-C₁-C₇-alkyl-)carbamoyl and N-mono- or N,N-di-(C₁-C₇-alkyl-, phenyl-, naphthyl-, phenyl-C₁-C₇-alkyl- or naphthyl-C₁-C₇-alkyl-)sulfamoyl, cyano, C₁-C₇-alkyl and substituted or unsubstituted heterocyclyl.

8. A compound of the formula I according to any of the preceding claims, wherein
R⁵ is methyl, isobutyl, tetrahydropyranyl or pyrazinyl wherein each is unsubstituted or substituted by one to three, substitutents selected from the group consisting of phenyl, hydroxyl, methyl or tetrahydropyranyl

9. A compound of the formula I according to any of the preceding claims, wherein
X is CH₂.

10. A compound of the formula I according to any of the preceding claims, wherein
X is O.

11. A compound of the formula I according to any of the preceding claims, wherein
Y is —C(O)—.

12. A compound of the formula I according to any of the preceding claims, wherein
Y is —C(O)O—.

13. A compound of the formula I according to any of the preceding claims, wherein
Ar is phenyl, naphthyl, indolyl, benzimidazolyl, benzofuranyl, quinolinyl, preferably phenyl or indolyl, wherein each is unsubstituted or substituted by one or more, e.g. up to three, substitutents selected from the group consisting of:
a substitutent of the formula —(C₀-C₇-alkylene)-(X)_{X}—(C₁-C₇-alkylene)-(Y)_{X}—(C₀-C₇-alkylene)-H where C₀-alkylene means that a bond is present instead of bound alkylene, r and s, each independently of the other, are 0 or 1 and each of X and Y, if present and independently of each other, is —O—, —NV—, —S—, —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, or is phenyl, naphthyl, phenyl- or naphthyl-C₁-C₇-alkyl and halo-C₁-C₇-alkyl; where said substituent of formula —(C₀-C₇-alkylene)-(X)ᵣ—(C₁-C₇-alkylene)-(Y)ₛ—(C₀-C₇-alkylene)-His preferably C₁-C₇-alkyl, hydroxy-C₁-C₇-alkyl, C₁-C₇-alkoxy-C₁-C₁-alkyl, C₁-C₇-alkoxy-C₁-C₇-alkoxy-C₁-C₇-alkyl, C₁-C₇-alkanoyloxy-C₁-C₇-alkyl, amino-C₁-C₇-alkyl, such as aminomethyl, (N—) mono- or (N,N-) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇alkoxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkoxy-carbonyl, halo-C₁-C₇-alkoxycarbonyl, hydroxy-C₁-C₇-alkoxycarbonyl, C₁-C₇-alkoxy-C₁-C₇-alkoxycarbonyl, amino-C₁-C₇-alkoxycarbonyl, (N—) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl and N-mono- or N,N-di-(C₁-C₇-alkyl)-aminosulfonyl.

14. A compound of the formula I according to any of the preceding claims, having the formula IA, 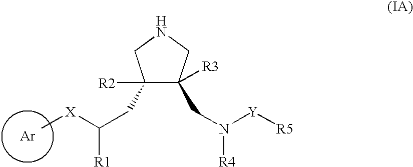
wherein R¹, R², R³, R⁴, R⁵, X, Y and Ar are as defined in any one of the preceding claims, or a pharmaceutically acceptable salt thereof.

15. A compound of the formula I according to any of the preceding claims, having the formula IB, 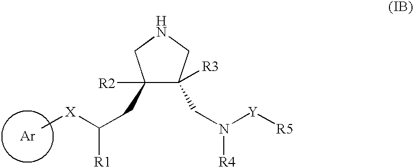
the formula IC, 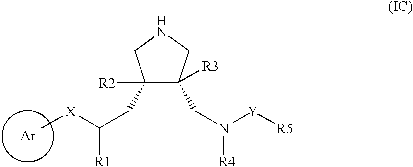
or the formula ID, 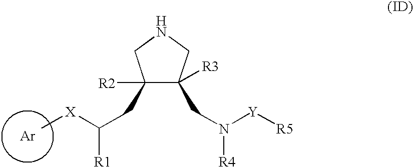
wherein R¹, R², R³, R⁴, R⁵, X, Y and Ar are as defined in any one of the preceding claims, or a pharmaceutically acceptable salt thereof.

16. A compound of the formula I, or a pharmaceutically acceptable salt thereof, according to any of the preceding claims for use in the diagnostic or therapeutic treatment of a warm-blooded animal.

17. A compound of the formula I, or a pharmaceutically acceptable salt thereof, according to any of the preceding claims for use according to claim 16 in the treatment of a disease that depends on activity of renin.

18. The use of a compound of the formula I, or a pharmaceutically acceptable salt thereof, according to any one of claims 1 to 16 for the manufacture of a pharmaceutical composition for the treatment of a disease that depends on activity of renin.

19. The use of a compound of the formula I, or a pharmaceutically acceptable salt thereof, according to any one of claims 1 to 16 for the treatment of a disease that depends on activity of renin.

20. A pharmaceutical formulation, comprising a compound of the formula I, or a pharmaceutically acceptable salt thereof, according to any one of claims 1 to 16 and at least one pharmaceutically acceptable carrier material.

21. A method of treatment of a disease that depends on activity of renin, comprising administering to a warm-blooded animal, especially a human, in need of such treatment a pharmaceutically effective amount of a compound of the formula I, or a pharmaceutically acceptable salt thereof, according to any one of claims 1 to 16.

22. A process for the manufacture of a compound of the formula I, or a pharmaceutically acceptable salt thereof, according to any one of claims 1 to 17, comprising:
reacting an acid of the formula II, 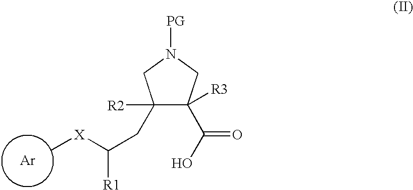
or a reactive derivative thereof, wherein R¹, R², R³, X, and Ar are as defined for a compound of the formula I in any one of claims 1 to 17 and PG is a protecting group, with
(i) an amino compound of the formula ill,
R4(R5Y)RNH (III)
wherein R⁴, R⁵ and Y are as defined for a compound of the formula I in any one of claims 1 to 17, under condensation conditions and
reducing the carbonyl group in the resulting compound of the formula IV 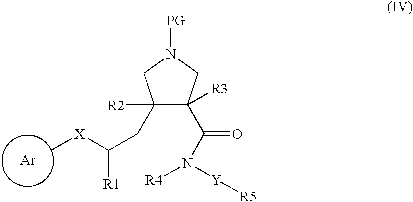
wherein R¹, R², R³, R⁴, R⁵, X, Y, Ar and PG are as defined for compounds of formulae II and III, to a methylene group, to obtain, upon removal of the protecting group PG, a compound of the formula I wherein R¹, R², R³, R⁴, R⁵, X, Y and Ar are as defined in any one of claims 1 to 17;
or
(ii) with an amino compound of formula V,
R⁴—NH₂ (V)
wherein R⁴ is as defined for a compound of the formula I in any one of claims 1 to 17, to give a compound of formula VI, 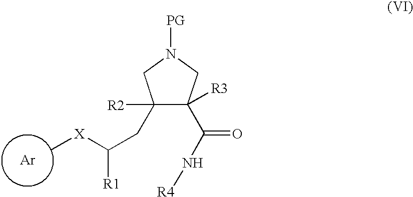
wherein R¹, R², R³, R⁴, X and Ar are as defined for a compound for formula I in any one of claims 1 to 17 and PG is a protecting group, and
reducing the carbonyl group whereby a compound of the formula VII 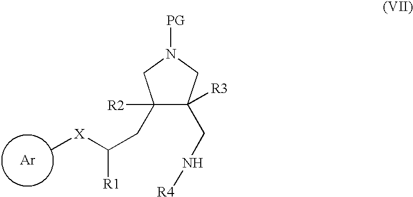
is obtained wherein R¹, R², R³, R⁴, X, Ar and PG are as defined for a compound of the formula VI, and reacting the compound of the formula VII with a compound of the formula VIII,
R5-Y-Z (VIII)
wherein R⁵ and Y are as defined for a compound of the formula I in any one of claims 1 to 17 and Z is a leaving group, to obtain, upon removal of the protecting group PG, the corresponding compound of the formula I, wherein R¹, R², R³, R⁴, R⁵, X, Y and Ar are as defined in any one of claims 1 to 17.

23. A process for the manufacture of a compound of the formula I, or a pharmaceutically acceptable salt thereof, according to any one of claims 1 to 17, comprising:
reacting an aldehyde of the formula IX, 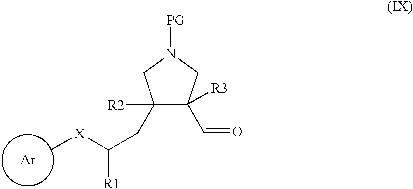
wherein R¹, R², R³, X and Ar are as defined for a compound of the formula I in any one of claims 1 to 17 and PG is a protecting group, either
(i) with an amino compound of the formula III, wherein R⁴, R⁵ and Y are as defined for a compound of the formula I in any one of claims 1 to 17, under conditions for reductive amination and, to obtain, upon removal of the protecting group PG, a compound of the formula I wherein R¹, R², R³, R⁴, R⁵, X, Y and Ar are as defined in any one of claims 1 to 17. or
(ii) with an amino compound of the formula V, wherein R⁴ is as defined for a compound of the formula I in any one of claims 1 to 17, whereby a compound of the formula VII 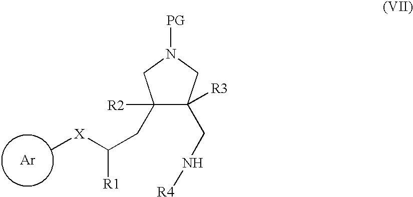
is obtained, wherein R¹, R², R³, R⁴, X, and Ar are as defined for a compound of the formula I in any one of claims 1 to 17 and PG is a protecting group, under conditions of reductive amination
and then reacting the compound of the formula (VII) with a compound of the formula VIII, wherein R⁵ and Y are as defined for a compound of the formula I in any one o of claims 1 to 17 and Z is a leaving group, to obtain, upon removal of the protecting group PG, a compound of formula I wherein R¹, R², R³, R⁴, R⁵, X, Y and Ar are as defined in any one of claims 1 to 17.

24. A process for the manufacture of a compound of the formula I, or a pharmaceutically acceptable salt thereof, wherein R¹ is hydroxyl, according to any one of claims 1 to 17, comprising:
i) oxidizing a a compound of the formula X, 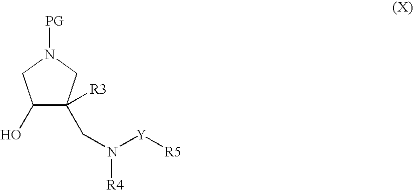
wherein R³, R⁴, R⁵, and Y are as defined for a compound of the formula I in any one of claims 1 to 17 and PG is a protecting group, to obtain a compound of formula XI 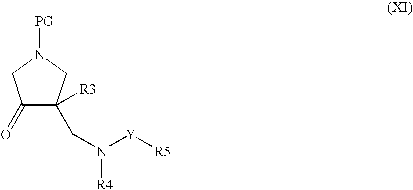
wherein R³, R⁴, R⁵ and Y are as defined for a compound of the formula I in any one of claims 1 to 17 and PG is a protecting group;
ii) reacting the compound of formula XI with a metallo reagent of the formula XII,
Ar—X—CHR¹—CH₂—Mg-Hal (XII)
wherein R¹, Ar and X are as defined for a compound of the formula I in any one of claims 1 to 17 and Hal is halo, to obtain, upon removal of the protecting group PG, the corresponding compound of the formula I wherein R¹, R², R³, R⁴, R⁵, X, Y and Ar are as defined in any one of claims 1 to 17.

25. A process according any one of claims 22 to 24 wherein, subsequent to any one or more of the processes mentioned;
converting an obtainable compound of the formula I or a protected form thereof into a different compound of the formula I;
converting a salt of an obtainable compound of formula I into the free compound or a different salt;
converting an obtainable free compound of formula I into a salt thereof, and/or separating an obtainable mixture of isomers of a compound of formula I into individual isomers;

26. A process according any one of claims 22 to 25 where in any of the starting materials, in addition to the specific protecting groups PG, further protecting groups may be present, and any protecting groups are removed at an appropriate stage in order to obtain the corresponding compound of the formula I, or a salt thereof.
